(19) United States
(12) Patent Application Publication
(10) Pub. No.: US 2025/0092358 A1
(43) Pub. Date: Mar. 20, 2025

(54) In Vitro Breeding Systems and Methods in Livestock

(71) Applicant: ABS Global, Inc., DeForest, WI (US)

(72) Inventors: Benjamin Beaton, DeForest, WI (US); Rebecca Lynn Krisher, Cottage Grove, WI (US); Chang Hyun Lee, Middleton, WI (US); Sandeep Rajput, Sun Prairie, WI (US)

(73) Assignee: ABS Global, Inc., DeForest, WI (US)

(21) Appl. No.: 18/562,222

(22) PCT Filed: May 26, 2022
(86) PCT No.: PCT/US22/31210

§ 371 Date: Nov. 17, 2023

Related U.S. Application Data
(60) Provisional application No. 63/194,667, filed on May 28, 2021.

(51) Int. Cl. C12N 5/073; A01K 67/02; C12N 5/0735; C12N 5/075; C12N 5/076

(52) U.S. Cl. CPC C12N 5/0604; A01K 67/02; C12N 5/0609; C12N 5/061; C12N 5/0611; C12N 2500/38; C12N 2501/11; C12N 2501/115; C12N 2501/119; C12N 2501/125; C12N 2501/13; C12N 2501/155; C12N 2501/16; C12N 2501/235; C12N 2501/385; C12N 2501/392; C12N 2501/415; C12N 2501/724; C12N 2501/727; C12N 2506/02; C12N 2513/00

(57) ABSTRACT

The present teachings provide for a method of breeding livestock in vitro. Provided are steps to create embryonic stem cells from a plurality of blastocysts, genotype the embryonic stem cells to select the best embryos for mating to create offspring, and induce the embryonic stem cells into primordial germ cell-like cells (PGCLCs). Male PGCLCs are further induced into spermatogonial stem cell-like cells, and then spermatid-like cells. Female PGCLCs are induced into oocytes, which are then matured. The resulting gametes can then be mixed with each other or with opposite sex gametes from animals with desirable genetics to create the next generation of embryos, which can then be run through the process again. This method of in vitro breeding can be used to increase the speed of genetic progress in livestock.

### CROSS REFERENCE TO PRIORITY APPLICATION

This application claims the benefit of and priority to U.S. Provisional Application 63/194,667, filed on May 28, 2021. 63/194,667 is hereby incorporated by reference in its entirety.

### INCORPORATION OF SEQUENCE LISTING

The Sequence Listing, including the file named RB-29-2021-WO1-SEQLST.xml, which is 33,957 bytes in size, was created on May 23, 2022 and is hereby incorporated by reference in its entirety.

### FIELD

The field of the invention is livestock embryonic stem cell derivation and differentiation.

### BACKGROUND

As the world population increases, food production will also need to increase. However, recent estimates are that global livestock production is responsible for about 15% of the world's greenhouse gas emissions (Capper, J. L. and Cady, R. A., J. Anim. Sci., 2020, 98, skz291). One way to increase food production while minimizing environmental impact is through genetic progress—increased efficiency of animal germplasm based on genetic predictions of animal performance. For example, dairy production increased nearly 25% between 2007 and 2017, despite the number of cattle decreasing by the same amount—that is, 75% of the dairy cattle present in 2007 made 25% more milk in 2017 than in 2007 (Capper, J. L. and Cady, R. A., J. Anim. Sci., 2020, 98, skz291). This increase in efficiency resulted in only a 1% increase in greenhouse gases despite the much larger increase in milk production. (Greenhouse gas emissions will shortly start to decrease because the dairy industry as a whole has committed to Net Zero carbon emissions by 2050.) Genetic improvement has also driven increases in pork production; production increased from 12.1 billion pounds of pork in 1959 to 22.8 billion pounds of pork in 2009. In the same time period, land use for pork production decreased 78%, water use decreased 41%, and the carbon footprint of pork production decreased 35%.

Several reviews have postulated that increasing genetic progress in dairy germplasm can be expedited through in vitro breeding—most notably Goszczynski, D. E., et al., Biology of Reproduction, 2019, 100, 885-895. Conventional breeding requires gestating an embryo for 6 (pigs) or 9 (cattle) months after an embryo is created in vitro, then waiting another 7 months (pigs) to a year (cattle) for the animal to reach sexual maturity. The present methods seek to shorten generation times using reproductive biology techniques that allow for increased genetic progress in shorter periods.

### SUMMARY

The present inventors have developed methods of breeding livestock such as cattle and pigs in vitro. In some embodiments, the present teachings provide for a method of livestock breeding that can comprise, consist of, or consist essentially of: A) selecting at least one livestock parent from a plurality of adult animals; B) obtaining a plurality of gametes from the at least one livestock parent; C) creating at least one embryo using the plurality of gametes; D) creating lines of embryonic stem cells from the at least one embryo; E) genotyping the at least one embryo; F) selecting at least one embryonic stem cell line for breeding; G) inducing stem cells from at least one selected line to differentiate into gamete or gamete-like cells; and H) combining the gametes or gamete-like cells with opposite sex gametes or gamete-like cells to produce at least one next generation embryo. In some configurations, genotyping the at least embryo can comprise, consist of, or consist essentially of genotyping the embryonic stem cells made from the at least one embryo or genotyping a biopsy of the at least one embryo. In various configurations, the combining of gametes or gamete-like cells can be through in vitro fertilization or through intracytoplasmic sperm injection. In various configurations, the gametes of an opposite sex parent can be from an adult animal of high genetic merit or the gamete-like cells are from an opposite sex genotype embryonic stem cell line produced in a method of the present teachings.

In various configurations, a method of the present teachings can further comprise, consist of, or consist essentially of: I) creating an embryonic stem cell line from the at least one next generation embryo to create next generation embryonic stem cell lines; and J) repeating steps E-H on the next generation embryonic cell lines. In various configurations, the method can further comprise, consist of, or consist essentially of producing animals from at least one of the embryonic stem cell lines produced while breeding livestock according to a method of the present teachings. In various configurations, the parents can be bovine parents or porcine parents.

In various configurations, the creating a line of embryonic stem cells can comprise, consist of, or consist essentially of: administering to an embryo, a blastocyst derived therefrom, or a part thereof a WNT-signaling inhibitor, a GSK3 signaling inhibitor, a tankyrase inhibitor, a Rho-kinase (ROCK) inhibitor, an inducer of activin A signaling, or a combination thereof. In some configurations, the WNT-signaling inhibitor, if present, can be iwr-1, iwr-1-endo, iwp-2, Box5, iCRT3, sclerostin, dkk2, dkk1, LF3, CCT036477, FH535, cardamonin, IWP-L6, Wnt-C59, niclosamide, XAV-939, ICG-001, LGK-974, CP21R7, NCB-0846, PNU-74654, salinomycin, KY021 11, WIKI4, PRI-724, KYA1797K, 2,4-diamino-quinazoline, Ant 1.4Br, Ant 1.40, apicularen, bafilomycin, ETC-159, G007-LK, G244-LM, IWR, NSC668036, PKF1 15-584, pyrvinium, quercetin, shizokaol D, BC2059, or a combination thereof; the GSK3 inhibitor, if present, can be CHIR99021, AR-A 014418, A 1070722, SB 415286, TCS 2002, 3F8, TDZD 8, TC-G 24, BIO-acetoxime, indirubin-3′-oxime, TWS 119, TCS 21311, SB 216763, BIO, lithium carbonate, kenpaullone, alsterpaullone, or CHIR 98014; the tankyrase inhibitor, if present, can be XAV939, AZ 6102, JW 55, TC-E 5001, or WIKI4; the ROCK inhibitor, if present, can be Y-27632 or PD0325901; and the inducer of activin A signaling, if present can be activin A, alantolactone, BMP4, nodal, stauprimide, SB-431542, A01, alantolactone, GDF8, or conophylline. In various configurations, the WNT-signaling inhibitor, if present, can be iwr-1, iwr-1-endo, iwp-2, Box5, iCRT3, sclerostin, dkk2, dkk1, LF3, CCT036477, FH535, cardamonin, IWP-L6, Wnt-C59, niclosamide, XAV-939, ICG-001, LGK-974, CP21R7, NCB-0846, PNU-74654, salinomycin, KY021 11, WIKI4, PRI-724, KYA1797K, 2,4-diamino-quinazoline, Ant 1.4Br, Ant 1.40, apicularen, bafilomycin, ETC-159, G007-LK, G244-LM, IWR, NSC668036, PKF1 15-584, pyrvinium, quercetin, shizokaol D, BC2059, or a combination thereof. In various configurations, the GSK3 inhibitor, if present, can be CHIR99021, AR-A 014418, A 1070722, SB 415286, TCS 2002, 3F8, TDZD 8, TC-G 24, BIO-acetoxime, indirubin-3′-oxime, TWS 119, TCS 21311, SB 216763, BIO, lithium carbonate, kenpaullone, alsterpaullone, or CHIR 98014. In various configurations, the tankyrase inhibitor, if present, can be XAV939, AZ 6102, JW 55, TC-E 5001, and WIKI4; the ROCK inhibitor, if present, can be Y-27632 or PD0325901; and the inducer of activin A signaling, if present is activin A, alantolactone, BMP4, nodal, stauprimide, SB-431542, A01, alantolactone, GDF8, or conophylline. In various configurations, the cells can be further treated with bFGF, FGF2, FGF4, or SUN 11602. In various configurations, the method can further comprise administering to the cells: vitamin C, an inducer of LIF signaling, or a combination thereof.

In various configurations, the inducing cells into gametes or gamete-like cells can comprise, consist of, or consist essentially of inducing cells from each of the selected stem cell lines into progenitor cells by administering to the cells an inducer of activin A signaling, an inducer of basic fibroblast growth factor (bFGF) signaling, an inducer of insulin signaling, an inducer of WNT signaling, or a combination thereof. In some configurations, the progenitor cells can be mesoderm-like cells and the inducing can comprise, consist of, or consist essentially of culturing the cells in media comprising activin A, CHIR99021, and a serum replacement. In various configurations, the progenitor cells can be formative cell-like cells and the inducing can comprise culturing the cells in media comprising bFGF, activin A, and CHIR99021. In various configurations, the inducing cells into gamete or gamete-like cells can further comprise, consist of, or consist essentially of inducing the progenitor cells into primordial germ cell-like cells (PGCLCs) by administering to the cells: an inducer of BMP signaling, an inducer of LIF signaling, an inducer of SCF signaling, an inducer of EGF signaling, an inducer of WNT signaling, or a combination thereof. In some configurations, the inducing the progenitor cells into PGCLCs can comprise, consist of, or consist essentially of administering to the cells LIF, SCF, EGF, and BMP4. In some configurations, the inducing can further comprise, consist of, or consist essentially of administering a serum replacement.

In various configurations, the inducing cells into gamete or gamete-like cells can comprise, consist of, or consist essentially of inducing the embryonic stem cells into spermatogonial stem cell-like cells (SSCLC) by administering to the cells an inducer of retinoic acid (RA) signaling, an inducer of Glial Cell Derived Neurotrophic Factor (GDNF) signaling, an inducer of testosterone signaling, or a combination thereof. In various configurations, the inducing embryonic stem cells into gamete or gamete-like cells can comprise inducing the embryonic stem cells into spermatids by administering an inducer of insulin signaling, an inducer of testosterone signaling, an inducer of FSH, Bovine Pituitary Extract (BPE), or a combination thereof. In various configurations, the inducing cells into gamete or gamete-like cells can comprise, consist of, or consist essentially of inducing the embryonic stem cells into oocytes by: placing the embryonic stem cells in a reconstituted ovary; administering to the cells in the reconstituted ovary an estrogen receptor agonist; administering to the cells in the reconstituted ovary at least one inducer of TGFβ signaling; and administering to the cells in the reconstituted ovary an inducer of FSH signaling, an inducer of EGF signaling, an inducer of gonadotropin signaling, or a combination thereof. In various configurations, the inducing the stem cells into gametes or gamete-like cells can comprise, consist of, or consist essentially of inducing the stem cells into oocytes by causing the cells to express FIGLA, SOHLH1, LHX8, NOBOX, STAT3, TBPL2, DYNLL1, SUB1, or a combination thereof.

In various embodiments, a method of cattle breeding of the present teachings can comprise, consist of, or consist essentially of A) selecting at least one bovine parent from a plurality of adult animals; B) obtaining a plurality of gametes from the at least one bovine parent; C) creating at least one embryo from the plurality of gametes; D) creating one or more lines of embryonic stem cells from the at least one embryo wherein the creating can comprise, consist of, or consist essentially of: i) administering to an embryo, a blastocyst matured therefrom, or a part thereof a WNT-signaling inhibitor and an inducer of FGF signaling; E) genotyping the one or more lines of embryonic stem cells; F) selecting combinations of stem cell lines for breeding; G) inducing stem cells from each selected line to differentiate into gametes or gamete-like cells wherein the inducing can comprise, consist of, or consist essentially of i) inducing the stem cells into progenitor cells by administering an inducer of activin A signaling, an inducer of bFGF signaling, an inducer of insulin signaling, an inducer of WNT signaling, or a combination thereof; ii) inducing the progenitor cells into PGCLCs by administering an inducer of BMP signaling, an inducer of LIF signaling, an inducer of SCF signaling, an inducer of EGF signaling, an inducer of WNT signaling, or a combination thereof; iii) inducing the PGCLCs that have an XY genotype into spermatid-like cells can comprise, consist of, or consist essentially of: a) inducing the PGCLCs into SSCLC by administering an inducer of RA signaling, an inducer of GDNF signaling, an inducer of testosterone signaling, or a combination thereof; b) inducing the SSCLCs into spermatid-like cells by administering an inducer of insulin signaling, an inducer of testosterone signaling, an inducer of FSH signaling, or BPE; iv) inducing the PGCLCs that have an XX genotype into oocytes which can comprise, consist of, or consist essentially of: a) placing the cells into a reconstituted ovary; b) administering to the reconstituted ovary an estrogen receptor agonist; c) administering to the reconstituted ovary at least one inducer of TGFβ signaling; d) administering to the reconstituted ovary an inducer of FSH signaling, an inducer of EGF signaling, an inducer of gonadotropin signaling, or a combination thereof; H) performing in vitro fertilization with the gametes or gamete-like cells to form a plurality of next generation embryos; I) creating an embryonic stem cell line from each of the plurality of next generation embryos to create next generation embryonic stem cell lines; and J) repeating steps E-H on the next generation embryonic cell lines. In some configurations, step D) can comprise, consist of, or consist essentially of incubating the embryo, blastocyst matured therefrom, or a part thereof in a medium comprising FGF2 and IWR1; step G) can comprise, consist of, or consist essentially of: i) inducing the stem cells into formative cells by administering activin A, bFGF, and CHIR99021 or inducing the stem cells into mesoderm-like cells by administering serum replacement, activin A, and CHIR99021; ii) inducing the formative cells or mesoderm-like cells into PGCLCs by administering to the cells BMP4, LIF, SCF, EGF, and a serum replacement; iii) inducing the XY PGCLCs into spermatid-like cells can comprise, consist of, or consist essentially of: a) inducing the PGCLCs into SSCLs by administering RA, GDNF, and testosterone; b) inducing the SSCLs into spermatid-like cells by administering serum replacement, testosterone, and BPE; iv) inducing the XX PGCLCs into oocytes can comprise, consist of, or consist essentially of: a) mixing the XX PGCLS with female bovine gonadal somatic cells and RA to form a reconstituted ovary; b) administering to the reconstituted ovary ICI182780 to induce secondary follicle-like structures (2FLs); c) administering to the 2FLs FSH, BMP15, and GDF9 to induce cumulus-oocyte complexes (COCs); d) administering to the COCs FSH, EGF, and hCG to form oocytes. In various configurations, the method can further comprise, consist of, or consist essentially of producing an animal from at least one of the embryonic stem cell lines created in step D or step I.

In various embodiments, a method of porcine breeding according to the present teachings can comprise, consist of, or consist essentially of: A) selecting at least one porcine parent from a plurality of adult animals; B) obtaining a plurality of gametes from the at least one porcine parent; C) creating at least one embryo from the plurality of gametes; D) creating one or more lines of embryonic stem cells from of the at least one embryo wherein the creating can comprise, consist of, or consist essentially of: i) isolating an embryo, a blastocyst matured therefrom, or a part thereof from the plurality of embryos; and ii) incubating the embryo, blastocyst matured therefrom, or part thereof in media supplemented with a GSK3 inhibitor, a tankyrase inhibitor, vitamin C, an activin A signaling inducer, a LIF signaling inducer, an FGF signaling inducer, or a combination thereof; E) genotyping each of the one more lines of embryonic stem cells; F) selecting combinations of stem cell lines for breeding; G) inducing stem cells from each selected line to differentiate into gamete-like cells wherein the inducing can comprise, consist of, or consist essentially of: i) inducing the stem cells into progenitor cells by administering to the cells an inducer of activin a signaling, an inducer of bFGF signaling, an inducer of insulin signaling, an inducer of WNT signaling, or a combination thereof; ii) inducing the progenitor cells into PGCLCs by administering to the cells an inducer of BMP signaling, an inducer of LIF signaling, an inducer of SCF signaling, an inducer of EGF signaling, an inducer of WNT signaling, or a combination thereof; iii) inducing the PGCLCs that have an XY genotype into spermatid-like cells comprising: a) inducing the PGCLCs into SSCLC by administering to the cells an inducer of RA signaling, an inducer of GDNF signaling, an inducer of testosterone signaling, or a combination thereof; b) inducing the SSCLCs into spermatid-like cells by administering to the cells an inducer of insulin signaling, an inducer of testosterone signaling, an inducer of FSH signaling, and BPE; iv) inducing the PGCLCs that have an XX genotype into oocytes comprising: a) placing the cells into a reconstituted ovary; b) administering to the reconstituted ovary an estrogen receptor agonist; c) administering to the reconstituted ovary at least one inducer of TGFβ signaling; d) administering to the cells an inducer of FSH signaling, an inducer of EGF signaling, an inducer of gonadotropin signaling, or a combination thereof; H) performing in vitro fertilization with the gametes or gamete-like cells to form a plurality of next generation embryos; I) creating an embryonic stem cell line from each of the plurality of next generation embryos to create next generation embryonic stem cell lines; and J) repeating steps E-H on the next generation embryonic cell lines. In some configurations, Step D) can comprise, consist of, or consist essentially administering to the embryo, blastocyst matured therefrom, or a part thereof bFGF, CHIR99021, and PD0325901; step G) can comprise, consist of, or consist essentially of: i) inducing the stem cells into formative cells by administering to the cells activin A, bFGF, and CHIR99021 or inducing the stem cells into mesoderm-like cells by administering to the cells serum replacement, activin A, and CHIR99021; ii) inducing the formative cells or mesoderm-like cells into PGCLCs by administering BMP4, LIF, SCF, EGF, and a serum replacement; iii) inducing the XY PGCLCs into spermatids can comprise, consist of, or consist essentially of: a) inducing the PGCLCs into SSCLs by administering RA, GDNF, and testosterone; b) inducing the SSCLs into spermatids by administering a serum replacement, testosterone, and BPE; iv) inducing the XX PGCLCs into oocytes comprising: a) mixing the XX PGCLCs with female bovine gonadal somatic cells and RA to form a reconstituted ovary; b) administering to the reconstituted ovary ICI182780 to induce secondary follicle-like structures (2FLs); c) administering to the 2FLs FSH, BMP15, and GDF9 to induce COCs; d) administering to the COCs FSH, EGF, and hCG to form oocytes; e) maturing the oocytes into MII oocytes. In various configurations, the method can further comprise, consist of, or consist essentially of producing animals from at least one of the embryonic stem cell lines created in step D or step I.

In various embodiments, the present teachings provide for a method of generating bovine primordial germ cell-like cells in vitro that can comprise, consist of, or consist essentially of: administering to bovine embryonic stem cells an inducer of FGF signaling, an inducer of activin a signaling, and an inducer of WNT signaling to induce bovine formative cells or administering to bovine embryonic stem cells an inducer of activin A signaling, an inducer of WNT signaling, and a serum replacement to induce bovine mesoderm-like cells; and administering to the bovine formative cells or the bovine mesoderm-like cells an inducer of LIF signaling, an inducer of SCF signaling, an inducer of EGF signaling, and an inducer of BMP signaling to induce primordial germ cell-like cells. In some configurations, bovine formative cells are made and the inducer of WNT signaling can induce WNT by inhibiting GSK3. In various configurations, bovine formative cells are made and the inducer of activin A signaling can be activin A, alantolactone, BMP4, nodal, stauprimide, SB-431542, A01, alantolactone, GDF8, or conophylline, the inducer of FGF signaling can be bFGF, SUN 11602, sucralfate, or FGF-P, and the inducer of WNT signaling can be WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5. In various configurations, bovine mesoderm-like cells are made, and the inducer of activin A signaling can be activin A, alantolactone, BMP4, nodal, stauprimide, SB-431542, A01, alantolactone, GDF8, or conophylline, and the inducer of WNT signaling can be WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5. In some configurations, the inducer of LIF signaling can be LIF, the inducer of SCF signaling can be SCF, and the inducer of BMP signaling can be BMP4, BMP8b, BMP7, TGFβ, sb4 (2-[[(4-Bromophenyl)methyl]thio]benzoxazole), triamcinolone, isoliquiritigenin and, km11073, 4′-hydroxychalcon, or SVAK-12.

In various embodiments, the present teachings provide for a method of inducing bovine primordial germ cell-like cells (bPGCLCs) from bovine embryonic stem cells that can comprise, consist of, or consist essentially of: administering to the cells: i) bFGF, activin A, and CHIR99021 to form bovine formative cells; or ii) activin A, CHIR99021, and serum replacement to form bovine mesoderm-like cells; B) administering to the cells rLIF, SCF, EGF, BMP4, and serum replacement to produce bPGCLCs.

In some embodiments, the present teachings provide for a method of inducing porcine embryonic stem cells which can comprise, consist of, or consist essentially of: providing an embryo, blastocyst, or part thereof; administering to the embryo, blastocyst, or part thereof: an FGF signaling inducer, a WNT signaling inducer, a Rho-kinase (ROCK) inhibitor, and an activin A signaling inducer. In some configurations, the inducer of FGF signaling can be bFGF, SUN 11602, sucralfate, or FGF-P, the inducer of WNT signaling can be WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5, the ROCK inhibitor can be Y-27632 or PD0325901, and the inducer of activin A signaling can be activin A, alantolactone, BMP4, nodal, stauprimide, SB-431542, A01, alantolactone, GDF8, or conophylline. In various configurations, the inducer of FGF signaling can be bFGF, SUN 11602, sucralfate, or FGF-P. In various configurations, the inducer of WNT signaling can be WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5. In various configurations, the ROCK inhibitor can be Y-27632 or PD0325901. In various configurations, the inducer of activin A signaling can be activin A, alantolactone, BMP4, nodal, stauprimide, SB-431542, A01, alantolactone, GDF8, or conophylline. In various configurations, the inducer of WNT signaling can induce WNT by inhibiting GSK3. In various configurations, the inducer of FGF signaling can be bFGF, the inducer of WNT signaling can be CHIR99021, the ROCK inhibitor can be PD0325901, and the inducer of activin A signaling can be activin A.

In various embodiments, the present teachings provide for a method of culturing lab grown meat that can comprise: inducing embryonic stem cells of the present teachings into muscle cells, adipose cells, organ cells, or a combination thereof; and culturing the muscle cells, adipose cells, organ cells, or a combination thereof on an 3-dimensional structure.

The present teachings provide for a method of livestock breeding that can comprise: A) selecting at least one livestock parent from a plurality of adult animals; B) obtaining a plurality of gametes from the at least one livestock parent; C) creating a plurality of embryos using the plurality of gametes; D) creating lines of embryonic stem cells from two or more of the plurality of embryos; E) genotyping two or more of the plurality of embryos; F) selecting at least one embryonic stem cell line for breeding; G) inducing stem cells from at least one selected line to differentiate into gamete or gamete-like cells; and H) combining the gametes or gamete-like cells with opposite sex gametes or gamete-like cells to produce a plurality of next generation embryos.

In some configurations, genotyping two or more of the plurality of embryos can comprise genotyping the embryonic stem cells made from the two or more of the plurality of embryos. In various configurations, genotyping two or more of the plurality of embryos can comprise genotyping a biopsy of the two or more of the plurality of embryos. In various configurations, the combining of gametes or gamete-like cells can be through in vitro fertilization. In various configurations, the combining of gametes or gamete-like cells can be through intracytoplasmic sperm injection. In various configurations, the gametes of an opposite sex parent can be from an adult animal of high genetic merit. In some configurations, the gametes of an opposite sex parent can be from a sexually mature animal of high genetic merit. In various configurations, the gamete-like cells of an opposite sex parent can be the gamete-like cells produced from an opposite sex stem cell line. In various configurations, the gamete-like cells of an opposite sex parent can be from an opposite sex genotype embryonic stem cell line produced in step D. In various configurations, the gamete-like cells of an opposite sex parent can be from an opposite sex embryonic stem cell line produced in step G.

In various configurations, a method of the present teachings can further comprise I) creating an embryonic stem cell line from each of the plurality of next generation embryos to create next generation embryonic stem cell lines; and J) repeating steps E-H on the next generation embryonic stem cell lines. In various configurations, a method of the present teachings can further comprise performing nuclear transfer using an embryonic stem cell from one of the embryonic stem cell lines from step (C). In various configurations, a method of the present teachings can further comprise performing nuclear transfer using an embryonic stem cell from one of the embryonic stem cell lines from step (I). In various configurations, the parents are bovine parents. In various configurations, the parents are porcine parents.

In various configurations, the creating a line of embryonic stem cells can comprise i) isolating an inner cell mass (ICM) from an embryo from the plurality of embryos; and ii) incubating the ICM in media supplemented with a wnt-signaling inhibitor. In some configurations the wnt-signaling inhibitor can be iwr-1, iwr-1-endo, iwp-2, Box5, iCRT3, sclerostin, dkk2, dkk1, LF3, CCT036477, FH535, cardamonin, IWP-L6, Wnt-C59, niclosamide, XAV-939, ICG-001, LGK-974, CP21R7, NCB-0846, PNU-74654, salinomycin, KY021 11, WIKI4, PRI-724, KYA1797K, 2,4-diamino-quinazoline, Ant 1.4Br, Ant 1.40, apicularen, bafilomycin, ETC-159, G007-LK, G244-LM, IWR, NSC668036, PKF1 15-584, pyrvinium, quercetin, shizokaol D, BC2059, or a combination thereof. In some configurations, the wnt-signaling inhibitor can be iwr-1 or iwr-1-endo. In various configurations, the wnt-signaling inhibitor can be iwr-1 and the cells can be further treated with FGF2, FGF4, or SUN 11602.

In various configurations, the creating a line of embryonic stem cells can comprise i) isolating an inner cell mass (ICM) from an embryo from the plurality of embryos; and ii) incubating the ICM in media supplemented with GSK3 inhibitor, a tankyrase inhibitor, or a combination thereof. In some configurations, the GSK3 inhibitor can be CHIR99021, AR-A 014418, A 1070722, SB 415286, TCS 2002, 3F8, TDZD 8, TC-G 24, BIO-acetoxime, indirubin-3′-oxime, TWS 119, TCS 21311, SB 216763, BIO, lithium carbonate, kenpaullone, alsterpaullone, or CHIR 98014. In various configurations, the GSK3 inhibitor can be CHIR99021. In various configurations, the tankyrase inhibitor can be XAV939, AZ 6102, JW 55, TC-E 5001, or WIKI4. In various configurations, the tankyrase inhibitor can be XAV939. In various configurations, the method can further comprise administering to the cells vitamin C, an inducer of activin A signaling, an inducer of LIF signaling, or a combination thereof.

In various configurations, the inducing cells into gametes or gamete-like cells can comprise inducing cells from each of the selected stem cell lines into Epiblast Like Cells (EpiLCs). In various configurations, the inducing cells into EpiLCs can comprise administering to the cells an inducer of activin A signaling, an inducer of basic fibroblast growth factor (bFGF) signaling, an inducer of insulin signaling, or a combination thereof. In some configurations, the inducer of activin A signaling can be activin A, alantolactone, BMP4, nodal, stauprimide, SB-431542, A01, alantolactone, GDF8, or conophylline. In some configurations, the inducer of activin A signaling can be activin A. In various configurations, the inducer of bFGF signaling can be bFGF, SUN 11602, sucralfate, and FGF-P. In some configurations, the inducer of bFGF signaling can be bFGF. In various configurations, the inducer of insulin signaling can be insulin, zinc chloride, zinc nitrate, zinc bromide, zinc sulfate zinc sulfate-7 water, bpV, BMOV, vanadyl rosiglitazone, vanadyl trehalose, vanadyl metformin, vanadyl quercetin, demethylasterriquinone B1, and BRD 7552. In various configurations the inducer of insulin signaling can be a media additive containing insulin. In some configurations, the media additive can be a serum replacement.

In various configurations, the inducing cells into gamete or gamete-like cells can comprise inducing the embryonic stem cells into primordial germ cell-like cells (PGCLCs). In some configurations, the inducing comprises administering to the cells an inducer of BMP signaling, an inducer of LIF signaling, an inducer of SCF signaling, an inducer of EGF signaling, an inducer of WNT signaling, or a combination thereof. In some configurations the inducer of BMP signaling can be BMP4, BMP8b, BMP7, TGFβ, sb4 (2-[[(4-Bromophenyl)methyl]thio]benzoxazole), triamcinolone, isoliquiritigenin and, km11073, 4′-hydroxychalcon, or SVAK-12. In some configurations, the inducer of BMP signaling can be BMP4, BMP8b, or a combination thereof. In various configurations, the inducer of LIF signaling can be LIF. In various configurations, the inducer of SCF signaling can be SCF. In various configurations, the inducer of EGF signaling can be EGF, amphiregulin, EPR, or HB-EGF. In some configurations, the inducer of EGF signaling can be EGF. In various configurations, the inducer of WNT signaling can be WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5.

In some configurations, the inducing comprises administering to the cells an inducer of BMP signaling, an inducer of LIF signaling, an inducer of SCF signaling, an inducer of EGF signaling, or a combination thereof. In some configurations the inducer of BMP signaling can be BMP4, BMP8b, BMP7, TGFβ, sb4 (2-[[(4-Bromophenyl)methyl]thio]benzoxazole), triamcinolone, isoliquiritigenin and, km11073, 4′-hydroxychalcon, or SVAK-12. In some configurations, the inducer of BMP signaling can be BMP4, BMP8b, or a combination thereof. In various configurations, the inducer of LIF signaling can be LIF. In various configurations, the inducer of SCF signaling can be SCF. In various configurations, the inducer of EGF signaling can be EGF, amphiregulin, EPR, or HB-EGF. In some configurations, the inducer of EGF signaling can be EGF.

In some configurations, the inducing cells into gamete or gamete-like cells can comprise inducing the embryonic stem cells into spermatogonial stem cell-like cells (SSCLC). In some configurations, the inducing can comprise administering to the cells an inducer of retinoic acid (RA) signaling, an inducer of Glial Cell Derived Neurotrophic Factor (GDNF) signaling, an inducer of testosterone signaling, or a combination thereof. In some configurations the inducer of retinoic acid signaling can be retinoic acid, retinoic acid p-hydroxyanilide, 9-cis retinoic acid, BMP4, TTNPB, methoprene acid, EC23, 1-Methyl-2-oxindole, adapalene, CD437, AC261066, CD1530, Ch 55, AM 580, AM 80, AC 55649, BMS 961, BMS 753, CD 2314, fenretinide, adapalene, EC 19, or SR 1078. In some configurations, the inducer of retinoic acid signaling can be retinoic acid. In various configurations, the inducer of GDNF signaling can be GDNF, Neurturin (NRTN), Artemin (ARTN), Persephin (PSPN), XIB4035, BT13, BT18, or BT44. In some configurations, the inducer of GDNF signaling can be GDNF. In various configurations, the inducer of testosterone signaling can be testosterone, methyltestosterone, fluoxymesterone, oxymetholone, oxandrolone, ethylestrenol, stanozolol, danazol, methandrostenolone, norethandrolone, GDNF, dihydrotestosterone, or icariin.

In various configurations, the inducing embryonic stem cells into gamete or gamete-like cells can comprise inducing the embryonic stem cells into spermatids. In various configurations, the inducing the cells can comprise administering an inducer of insulin signaling, an inducer of testosterone signaling, an inducer of FSH signaling, Bovine Pituitary Extract (BPE), or a combination thereof. In various configurations, the inducer of insulin signaling can be insulin, bpV, BMOV, vanadyl rosiglitazone, vanadyl trehalose, vanadyl metformin, vanadyl quercetin, demethylasterriquinone B1, and BRD 7552. In various configurations, the inducer of insulin signaling can be insulin. In various configurations, the inducer of testosterone signaling can be testosterone, GDNF, dihydrotestosterone, and icariin. In various configurations, the inducer of testosterone signaling can be testosterone. In various configurations, the inducer of FSH signaling is selected from the group consisting of FSH, corifollitropin alfa, FSHβ-CTP-α, N2-α FSH, pregnant mare's serum gonadotropin, thiazolidinone, and hexahydroquinoline.

In various configurations, the inducing cells into gamete or gamete-like cells can comprise inducing the embryonic stem cells into oocytes. In some configurations, the inducing embryonic stem cells into oocytes can comprise placing the embryonic stem cells in a reconstituted ovary. In various configurations, the inducing embryonic stem cells into oocytes can comprise administering to the cells an estrogen receptor agonist. In some configurations, the estrogen receptor can be ICI182780, ferutinin, estropipate, estrone-3-sulfate, S-equol, WAY-200070, DY 131, GSK 4716, AB-1, ERB-041, biochanin A, β-Estradiol, α-Estradiol, PPT, DPN, FERb 033, or AC 186. In various configurations, the estrogen receptor agonist can be ICI182780. In various configurations, the inducing can comprise administering to the cells at least one inducer of TGFβ signaling. In some configurations, the at least one inducer of TGFβ signaling can be selected from the group consisting of BMP15, BMP4, BMP8b, BMP7, GDF9, TGFβ, sb4 (2-[[(4-Bromophenyl)methyl]thio]benzoxazole), triamcinolone, isoliquiritigenin and, km11073, PD07824, 4′-hydroxychalcon, or SVAK-12. In some configurations, the at least one inducer of TGFβ signaling can be BMP15, GDF9 or a combination thereof. In various configurations, the inducing the cells into oocytes can comprise administering to the cells an inducer of FSH signaling, an inducer of EGF signaling, an inducer of gonadotropin signaling, or a combination thereof. In some configurations, the inducer of FSH signaling can be FSH, corifollitropin alfa, FSHβ-CTP-α, N2-α FSH, pregnant mare's serum gonadotropin, thiazolidinone, or hexahydroquinoline. In some configurations, the inducer of FSH signaling can be FSH. In various configurations, the inducer of EGF signaling can be EGF, amphiregulin, EPR, and HB-EGF. In some configurations, the inducer of EGF signaling can be EGF. In various configurations, the inducer of gonadotropin signaling can be hCG, chorionic gonadotropin, choriogonadotropin alfa, or corifollitropin alfa. In some configurations, the inducer of EGF signaling can be EGF.

In various configurations, the inducing can comprise causing the cells to express the FIGLA, SOHLH1, LHX8, NOBOX, STAT3, TBPL2, DYNLL1, or SUB1 genes, or a combination thereof. In some configurations the causing the cells to express the genes can comprise transiently transfecting vectors comprising the genes into the cells.

In various configurations, a method of inducing ESCs into directly induced oocyte like cells can comprise causing the cells to express the FIGLA, SOHLH1, LHX8, NOBOX, STAT3, TBPL2, DYNLL1, or SUB1 genes, or a combination thereof. In various configurations, the causing the cells to express the genes can comprise transiently transfecting vectors comprising the genes into the cells.

In various embodiments, a method of cattle breeding in accordance with the present teachings can comprise: A) selecting at least one bovine parent from a plurality of adult animals; B) obtaining a plurality of gametes from the at least one bovine parent; C) creating a plurality of embryos from the plurality of gametes; D) creating a line of embryonic stem cells from each of the plurality of embryos wherein the creating can comprise: i) optionally isolating an ICM from an embryo from the plurality of embryos; and ii) incubating the ICM in media supplemented with a wnt-signaling inhibitor; E) genotyping each line of embryonic stem cells; F) selecting combinations of stem cell lines for breeding; G) inducing stem cells from each selected line to differentiate into gamete-like cells wherein the inducing can comprise: i) inducing the stem cells into EpiLC by administering an inducer of activin A signaling, an inducer of bFGF signaling, an inducer of insulin signaling, or a combination thereof; ii) inducing the EpiLCs into PGCLCs by administering an inducer of BMP signaling, an inducer of LIF signaling, an inducer of SCF signaling, an inducer of EGF signaling, an inducer of WNT signaling, or a combination thereof; iii) inducing the PGCLCs that have an XY genotype into spermatid-like cells comprising: a) inducing the PGCLCs into SSCLC by administering an inducer of RA signaling, an inducer of GDNF signaling, an inducer of testosterone signaling, or a combination thereof; b) inducing the SSCLCs into spermatid-like cells by administering an inducer of insulin signaling, an inducer of testosterone signaling, an inducer of FSH signaling, or BPE; iv) inducing the PGCLCs that have an XX genotype into oocytes comprising: a) placing the cells into a reconstituted ovary; b) administering to the reconstituted ovary an estrogen receptor agonist; c) administering to the reconstituted ovary at least one inducer of TGFβ signaling; d) administering to the reconstituted ovary an inducer of FSH signaling, an inducer of EGF signaling, an inducer of gonadotropin signaling, or a combination thereof; H) performing in vitro fertilization with the gametes or gamete-like cells to form a plurality of next generation embryos; I) creating an embryonic stem cell line from each of the plurality of next generation embryos to create next generation embryonic stem cell lines; and J) repeating steps E-H on the next generation embryonic cell lines. In some configurations, step D) can comprise incubating the ICM, an embryo, a blastocyst, or a part thereof in a medium comprising FGF2 and IWR1; step G) can comprise: i) inducing the stem cells into EpiLCs by administering activin A, bFGF, and serum replacement; ii) inducing the EpiLCs into PGCLCs by administering BMP4, LIF, SCF, BMP8b, and EGF; iii) inducing the XY PGCLCs into spermatid-like cells comprises: a) inducing the PGCLCs into SSCLs by administering RA, GDNF, and testosterone; b) inducing the SSCLs into spermatid-like cells by administering serum replacement, testosterone, and BPE; iv) inducing the XX PGCLCs into oocytes comprising: a) mixing the XX PGCLS with female bovine gonadal somatic cells and RA to form a reconstituted ovary; b) administering to the reconstituted ovary ICI182780 to induce secondary follicle-like structures (2FLs); c) administering to the 2FLs FSH, BMP15, and GDF9 to induce cumulus-oocyte complexes (COCs); d) administering to the COCs FSH, EGF, and hCG to form oocytes. In various embodiments, the method can further comprise performing nuclear transfer using a cell from at least one of the embryonic stem cell lines created in step D or step I.

In various embodiments, a method of porcine breeding according to the present teachings can comprise: A) selecting at least one porcine parent from a plurality of adult animals; B) obtaining a plurality of gametes from the at least one porcine parent; C) creating a plurality of embryos from the plurality of gametes; D) creating a line of embryonic stem cells from each of the plurality of embryos wherein the creating comprises: i) optionally isolating an ICM from an embryo from the plurality of embryos; and ii) incubating the ICM or an embryo, blastocyst matured therefrom, or a part thereof, in media supplemented with a GSK3 inhibitor, a tankyrase inhibitor, vitamin C, an activin A signaling inducer, a LIF signaling inducer, or a combination thereof; E) genotyping each line of embryonic stem cells; F) selecting combinations of stem cell lines for breeding; G) inducing stem cells from each selected line to differentiate into gamete-like cells wherein the inducing comprises: i) inducing the stem cells into EpiLC by administering to the cells an inducer of activin a signaling, an inducer of bFGF signaling, an inducer of insulin signaling, or a combination thereof; ii) inducing the EpiLCs into PGCLCs by administering to the cells an inducer of BMP signaling, an inducer of LIF signaling, an inducer of SCF signaling, an inducer of EGF signaling, an inducer of WNT signaling, or a combination thereof; iii) inducing the PGCLCs that have an XY genotype into spermatid-like cells comprising: a) inducing the PGCLCs into SSCLC by administering to the cells an inducer of RA signaling, an inducer of GDNF signaling, an inducer of testosterone signaling, or a combination thereof; b) inducing the SSCLCs into spermatid-like cells by administering to the cells an inducer of insulin signaling, an inducer of testosterone signaling, an inducer of FSH signaling, and BPE; iv) inducing the PGCLCs that have an XX genotype into oocytes comprising: a) placing the cells into a reconstituted ovary; b) administering to the reconstituted ovary an estrogen receptor agonist; c) administering to the reconstituted ovary at least one inducer of TGFβ signaling; d) administering to the cells an inducer of FSH signaling, an inducer of EGF signaling, and an inducer of gonadotropin signaling; H) performing in vitro fertilization with the gametes or gamete-like cells to form a plurality of next generation embryos; I) creating an embryonic stem cell line from each of the plurality of next generation embryos to create next generation embryonic stem cell lines; and J) repeating steps E-H on the next generation embryonic cell lines. In some configurations, Step D) can comprise incubating the ICM in a medium comprising CHIR99021, WH-4-023, XAV939, vitamin C, activin A, and LIF; step G) can comprise: i) inducing the stem cells into EpiLCs by administering activin A, bFGF, and serum replacement; ii) inducing the EpiLCs into PGCLCs by administering BMP4, LIF, SCF, BMP8b, and EGF; iii) inducing the XY PGCLCs into spermatids comprising: a) inducing the PGCLCs into SSCLs by administering RA, GDNF, and testosterone; b) inducing the SSCLs into spermatids by administering serum replacement, testosterone, and BPE; iv) inducing the XX PGCLCs into oocytes comprising: a) mixing the XX PGCLCs with female bovine gonadal somatic cells and RA to form a reconstituted ovary; b) administering to the reconstituted ovary ICI182780 to induce secondary follicle-like structures (2FLs); c) administering to the 2FLs FSH, BMP15, and GDF9 to induce COCs; d) administering to the COCs FSH, EGF, and hCG to form oocytes; e) maturing the oocytes into MII oocytes. In various configurations, the method can further comprise performing nuclear transfer using a cell from at least one of the embryonic stem cell lines created in step D or step I.

### BRIEF DESCRIPTION OF THE DRAWINGS

FIG. 1 illustrates bovine embryonic stem cells growing on MEF1 feeder cells.

FIG. 2 illustrates bovine embryonic stem cells growing on feeder free, vitronectin coated plates.

FIG. 3 illustrates that bovine embryonic stem cells of the present teachings form embryoid bodies.

FIG. 4 illustrates that bovine embryonic stem cells of the present teachings will differentiate into all three main cell types: neuronal cells (ectodermal cells), mesodermal cells, and endodermal cells.

FIG. 5 illustrates induction of feeder free bovine embryonic stem cells to primordial germ cell-like cells through an intermediate cell type.

FIG. 6 illustrates morphology of induced bovine primordial germ cell-like cells.

FIG. 7 illustrates porcine embryonic stem cells growing on CF1 mouse feeder cells.

FIG. 8 illustrates porcine embryonic stem cells growing on feeder free, vitronectin or fibronectin coated plates.

FIG. 9. illustrates that porcine embryonic stem cells of the present teachings will form embryo bodies.

FIG. 10 illustrates that porcine embryonic stem cells of the present teachings will spontaneously differentiate into mesodermal cells.

FIG. 11 illustrates that porcine embryonic stem cells of the present teachings will spontaneously differentiate into endodermal cells.

### DETAILED DESCRIPTION

The methods described herein comprise breeding methods that include the steps of selecting parents, performing a cross via IVF to form a plurality of embryos, creating embryonic stem cell (ESC) lines from those embryos, genotyping the cell lines, selecting parents for the next generation, deriving gametes from the selected embryonic stem cell lines, performing IVF or intracytoplasmic sperm injection (ICSI) with those gametes to form a plurality of embryos, then producing ESCs from those embryos to begin the process again. The methods disclosed herein include applications of breeding methods to various mammalian livestock species such as cattle, pigs, sheep, and goats. Cattle and pigs are particularly desirable. These methods require less time than naturally gestating offspring, genotyping them, then waiting for the animal to become sexually mature before mating. While it is less risky to create embryonic stem cell lines and then obtain a genotype, it is possible to biopsy an embryo and genotype it, then create embryonic stem cell lines from only selected embryos. Embryonic stem cell lines created during this process that have high genetic merit can be used to create animals for production (of milk, meat, or gametes) using standard nuclear transfer protocols.

Also disclosed herein are the first primordial germ cell-like cells derived from bovine embryonic stem cells.

### Animal Selection

### Bovine

The cattle industry uses genomic information (often referred to in the industry as genetics), including the genotype of various small nucleotide polymorphisms (SNPs) to predict the value of a particular animal based on past results with animals of similar genetics at a particular locus.

As used herein, “locus”, or plural “loci”, refers to a physical site or location of a specific gene or marker on a chromosome. Loci may also be characterized as either ‘A’ loci, ‘B’ loci, or heterozygous ‘A/B’ loci. The identification of loci as either ‘A’ or ‘B’ loci is determined according to the top (TOP) and bottom (BOT) designations based on the polymorphism itself, or the contextual surrounding sequence as developed by ILLUMINA®, Inc. (San Diego, CA). Methods for determining the designation of a polymorphic site as ‘A’ or ‘B’ are known in the art, for example as provided by ILLUMINA®'s Technical Note entitled “‘TOP/BOT’ Strand and ‘A/B’ Allele”, available on the internet at www(dot)illumina(dot)com/documents/products/technotes/technote_topbot.pdf. The NCBI's dbSNP database adopted the TOP/BOT nomenclature in 2005 and the designation is well known to those of skill in the art. Through sequence comparison, extensive information about each locus is available to a person of ordinary skill in the art, including, but not limited to, dbSNP identifier, sources, chromosomal location, genes, transcripts, linkage to genes or quantitative trait loci (QTL), and interactions. Predicted Transmitting Abilities (PTAs) can be computed for various traits, for example in the broad categories of production (milk and milk components), health/fitness, and type. Dairy cattle are evaluated for the traits of milk, fat, and protein yield, length of productive life, and somatic cell score (an indicator of mastitis). Evaluation procedures combine information from relatives of an evaluated animal and from the animal itself in the case of cows. Additionally, numerous type or conformation traits are evaluated routinely. Traits are typically combined into an index based on their relative economic weights. For example, the Net Merit index (NM$) computed by USDA AIPL in conjunction with CDCB estimates lifetime profit based on incomes and expenses relevant for today's dairy producers and is expressed as a dollar value. Traits included in NM$ include: protein (lb.), fat (lb.), productive life, somatic cell score, udder composite, feet/legs composite, body size composite and daughter pregnancy rate. Holstein Association USA (HAU) calculates the Total Performance Index (TPI®; HAU, Brattleboro, Vermont). It includes the traits of protein, fat, type, udder composite, feet and leg composite, daughter pregnancy rate, productive life, somatic cell score, daughter calving ease, daughter stillbirth and dairy form. The JERSEY PERFORMANCE INDEX™ (JPI™, American Jersey Cattle Association, Reynoldsburg, Ohio), which is used by the American Jersey Cattle Association, is comprised of the following traits: protein, fat, functional trait index, productive life, somatic cell score, and daughter pregnancy rate. This functional trait index is based on the bull/cow PTAs for all type traits. The JPI™ is updated periodically in the interest of improving the breed. The Council of Dairy Cattle Breeding publishes estimated breeding values (eg NM$, CM$) three times a year—these publications are referred to as “Sire Summaries.” The predicted values disclosed within were current as of the December 2021 Sire Summary. Some cattle breeders also use custom indices. Trait parameters have been correlated with the underlying genetics and the heritability of each trait determined. The genetic and phenotypic correlations among the twelve PTA traits are also provided by VanRaden et al., 2018. These correlations between genotype and phenotype have been tracked over time, allowing for the calculation of reliability scores such as Net Merit Reliability (NM_Rel) and Milk Reliability (Milk_Rel) which are expressed as a percent.

Holstein Association USA (HAU) calculates the Total Performance Index (TPI®; HAU, Brattleboro, Vermont)). It includes the traits of protein, fat, type, udder composite, feet and leg composite, daughter pregnancy rate, productive life, somatic cell score, daughter calving ease, daughter stillbirth and dairy form. Like Net Merit scores, TPI® is calculated based on a comparison with a baseline average animal in 2010, however, the Holstein Association USA has not announced plans to update the baseline animal. Accordingly, as used herein, TPI® scores are calculated using a 2010 baseline animal. However, should a new baseline year be adopted, persons of ordinary skill in the art will be able to calculate or convert TPI® values based on the 2010 baseline population for any future baseline animal using methods known in the art (available on the internet at www.holsteinusa.com). TPI® (HAU, Brattleboro, Vermont) incorporates production, management, Type, and important linear and composite traits. TPI® focuses on dairies paid for protein plus fat and requiring more emphasis on Type.

Individual breeders and genetics companies also create their own indices and other custom calculations that can be used to aid with breeding decisions.

Beef cattle associations, such as the American Angus Association, publish similar reports, including value indexes such as beef value ($B) which expresses the predicted value of an animal carcass.

Trait indices can be used to choose the parents of the next generation. Skilled artisans are able to use selection and trait indices to choose animals to be parents to the next generation. Once parents are chosen, the male gametes are collected-methods of collecting ejaculates or epididymal semen are known in the art. Female gametes are also collected through art recognized methods, most frequently through ovum pick up.

Several other predicted traits based on SNP genotypes are described for embryonic stem cell lines disclosed herein. As used herein, daughter pregnancy rate (DPR) is a genetic measure of the percentage of non-pregnant cows or heifers eligible to become pregnant that actually become pregnant during each 21-day period (heat cycle). DPR is similar to, but not always the same as, pregnancy rates computed for herd management purposes. Daughters of sires which have larger PTA DPR are more likely to conceive during a given heat cycle and each 1% increase in PTA DPR is associated with a genetic decrease of 4 days open.

As used herein, Cow Livability (LIV) represents the probability value of a lactation not ending in death or euthanasia relative to the average of the breed. This trait is important because cows that die during lactation have no value and the farmer must pay to dispose of the carcass. The trait is similar to PL which includes cows culled from the herd for any reason. LIV values range from about −5 to +5, where 5% more of a bull's daughters will remain alive compared to the breed average. Similarly, heifer livability (HLIV) represents the expected livability percentage of an animal's female offspring from 2 days after birth to 18 months of age under average management conditions. Higher numbers are more favorable, and the trait is expressed relative to the breed average—e.g. a Holstein bull with a HLIV value of 1 would be expected to have 1% more of his daughters surviving to 18 months of age compared to the breed average.

As used herein, heifer conception rate (HCR) is the percentage of inseminated heifers that become pregnant at each service, shown as a deviation in percentage.

As used herein, cow conception rate (CCR) is the percentage of inseminated cows that become pregnant at each service, shown as a deviation in percentage.

As used herein, predicted transmitting ability milk (MILK or PTA MILK) is a yield trait for milk measured in pounds that is the predicted difference of the milk yield of the offspring from the average. MILK is shown as added pounds of milk expected per lactation for average daughters of individual sires. Higher numbers are preferred.

Residual feed intake (RFI) is defined as the difference between an animal's actual and expected feed intake, after accounting for size, production, and changes in body weight. This trait is expressed in pounds per lactation, and lower numbers are considered better. Feed saved (FSAV) is expressed in the pounds of food saved per lactation based on body weight and residual food intake.

As used herein, predicted transmitting ability type (PTAT or TYPE) is represented as differences in points from a base population physical conformation. Daughter final scores are collected by breed classifiers. Raw scores then are adjusted for cow age and used to derive Type PTAs (PTAT). These PTATs are represented as differences in points from the base population. TYPE values are normalized to enable comparisons across different base populations through time. Higher numbers correlate with more desirable physical conformations.

As used herein, STA dairy form (DF or DFM), formerly known as “dairy character”, refers to sharpness, angularity, flatness of bone, openness of rib and length of neck that provides an indication of “milkiness” and reflects the ability of a dairy cow to produce milk from the feed over flesh and fat.

Dairy cattle are evaluated and described using criteria generally known as linear descriptive traits that are well known in the art. These linear descriptive traits include Stature (STA), Strength (STR), Body Depth (BDE), Rump Angle (RPA), Thurl Position, Rump Width, Fore Udder Height (FTA), Fore Udder Attachment (FUA), Rear Udder Height (RUH), Rear Udder Width (RUW), Udder Cleft (UCL), Udder Depth (UDP), Front Teat Placement (FTP), Rear Teat Placement (RTP), Teat Length (TLG), Udder Tilt (UT), Rear Legs (Side View) (RLS), Rear Legs (Rear View) (RLR), Feet Leg Score (FLS), Foot Angle, Thurl Width (TRW), and Body Condition. These linear trait criteria are well known to those skilled in the art. See for example, The Dairy Cow Today: U.S. Trends, Breeding, and Progress Since 1980, S. L. Spahr and G. W. Opperman, Chapter 9, Type and classification and trait appraisal, hereby incorporated by reference in its entirety.

Linear descriptive traits are often combined into composite indexes to simplify the process of describing the transmitting pattern for type traits. Composite indexes include the feet & legs composite (FLC), the udder composite index (UDC), the body form (BF) composite index, body size composite (BSC) index, the dairy capacity (DC) composite index, and the body weight composite (BWC). The FLC composite is a combination of rear legs, side view and foot angle linear traits. The UDC incorporates the udder attachment, rear udder height, rear udder width, udder depth, udder cleft, front teat placement, and rear teat placement linear traits. The UDC is designed so that the association between UDC and herd life is maximized. Larger values are associated with longer herd life. The udder composite index describes a well formed capacious udder with strong attachment. Using breeding animals with a high UDC results in a lowering of the somatic cell score and daughters whose udders are trouble-free and capable of holding more milk. The BF index combines the linear traits of stature, body depth, rump angle, and rump width. The DC composite combines the linear traits of dairy form and strength. The BSC is another composite index calculated from four linear traits: stature, strength, body depth, and rump width. Every 1.0 STA increase in the BSC correlates with a 24 pound predicted increase in mature body weight. BWC is computed from various linear traits to predict body weight; these traits include stature, strength, dairy form and rump width (as of the August 2021 Sire Summary).

As used herein, “Productive Life” (PL) is a measure of how long dairy cows survive in a herd after they calve for the first time. It is based on calving dates, culling or death dates, and days in milk (based on dry dates) in each lactation for cows on DHI test. The PTA for Productive Life (PL) is expressed as additional months of life in the milking string. Bulls with larger PL are expected to sire daughters that have longer productive lives. Data used to compute PL include actual longevity, stage of lactation, and culling data supplemented with data from traits that are correlated with PL. By assigning the largest PL credits for months in peak production and by giving later lactations slightly more credit than first lactation, PL reflects the economic impact of cow longevity. The heritability of PL is low at 0.085 and the trait is expressed late in the life of dairy cow. Accordingly, PL is a difficult trait to improve through selection because of low heritability and expression of the trait late in life. Methods for calculating PL are known in the art. See VanRaden et al., Journal of Dairy Science 76:2758-2764 (1993), VanRaden et al., Journal of Dairy Science 78:631-638 (1995), Weigel et al., Journal of Dairy Science 81:2040-2044(1998).

As used herein, “Somatic Cell Score” (SCS) is calculated from the Somatic Cell Count (SCC). When milk is produced, a small number of cells are also transferred to the milk (along with the proteins, fat, water, and minerals that make up milk.) Although all milk contains some of these cells, milk quality is affected if they are present in very high numbers. Milk processors limit the concentration of cells that they will allow in milk they buy from farmers. Also, knowing the SCS for an individual cow can help the farmer tell if the cow is healthy because irritation in the udder can cause higher SCS. Health management has the biggest effect on SCS, but just like some people inherit a higher chance of getting ear infections, cows can inherit traits which cause higher SCS. Next to traits like milk or protein production, SCS has a low heritability. Somatic Cell Score PTA is calculated using somatic cell score data from the first five lactations as an indicator of mastitis resistance. Bulls with the lowest PTA SCS are expected to sire daughters with the lowest SCS, the lowest somatic cell counts (SCC), and the fewest cases of mastitis. The present disclosure provides for, and includes, reduced SCS in progeny compared to a dam parent.

As used herein, “Fertility Index” (FI) combines several reproductive components into one overall index: ability to conceive as a heifer, ability to conceive as a lactating cow, and a cow's overall ability to start cycling again, show heat, conceive, and maintain a pregnancy. The Fertility Index is derived from the formula: Fertility Index=18% Heifer Conception Rate (HCR)+18% Cow Conception Rate (CCR)+64% Daughter Pregnancy Rate (DPR).

As used herein, “Sire Calving Ease” (SCE) measures the tendency of calves from a particular sire to be born more or less easily and is expressed as a percent of difficult births in first calf heifers on a scale of 1 to 5 (1 is classified as “no problem”). The percent difficult birth among Holstein is about 8%. Generally, bulls having an SCE of 8% or less are considered “calving ease” bulls. Lower numbers are preferred.

As used herein, “Daughter Calving Ease” (DCE), like SCE, is a measurement of the tendency of calve from a particular animal to be born more or less easily. Lower numbers are preferred.

As used herein, “Service Sire Stillbirth” (SSB) expresses the proportion of stillborn calves expected from sires. The genetic base for Stillbirth is 8%.

As used herein, “Early First Calving” (EFC) is the opposite value of the number of days that a bull's progeny are expected to give birth that is different from the average. For example, if the bull's daughters give birth two days earlier than average (i.e. −2 days from average), his EFC would be +2.0.

As used herein, “Daughter Stillbirth” (DSB) is the tendency of calves from a sire to be stillborn and applies to the Holstein breed only. As discussed below, DSB can be related to certain haplotypes.

Breeders have developed merit measures for the evaluation of value of Bos taurus animals over the lifetime of offspring. Various merit measures account for the additional net profit that an offspring of an animal will provide over its lifetime. Income and expenses for a typical dairy operation have been estimated, so that a measure of overall net profit can be calculated. Three different values (Net, Fluid and Cheese) of lifetime profitability are available. The primary difference between the formulas is the emphasis that is placed on the components. When breeding, producers select the index that is closest to the milk payment in their area. Net merit is based upon the future anticipated average milk price for all of the United States. Fluid Merit would be for producers who do not receive any payment for protein. In the Fluid Merit formula, a negative value is placed on protein because additional feed is required to produce additional protein. Without a direct payment for the additional protein, this results in a negative value. Cheese Merit may be appropriate for farmers selling their milk directly to a cheese plant.

As used herein cheese merit (CM$) is an index that incorporates economic values appropriate for milk sold to be made into cheese or other dairy products. The formula incorporates MILK, PTAF, PTAP, and various health and type traits. A discussion of CM$ is available on us.altagenetics.com.

As used herein, a haplotype is a combination of alleles (DNA sequences) at different locations on a chromosome that are transmitted together as a group (linked). Haplotype tests are available that provide for the identification of recessive disorders that affect fertility and other traits. See Cole et al., “Haplotype tests for recessive disorders that affect fertility and other traits,” USDA AIP Research Report Genomic3 (09-13) updated Dec. 1, 2018. Certain haplotypes are undesirable in a Bos taurus germplasm as provided in the present specification. When the recessive haplotype is homozygous, fertility and other critical traits are significantly affected. The germplasm of the present disclosure can be used to improve herds and reduce the presence of these undesirable haplotypes.

Recessive haplotype mutations include polledness, which is lack of horns—haplotype HHP. At one time, this was considered an undesirable phenotype because it was closely associated with undesirable genetics. However, animal welfare requires de-homing animals to prevent injuries, and recent breeding advances have separated the trait from the associated poor production phenotypes, making it a more desirable trait. Another recessive haplotype is red coat color, which is only undesirable because Holsteins are supposed be black and white; red coat color can be caused by haplotypes HBR, HDR, and HHR. In contrast, several haplotypes are undesirable because they do not produce viable homozygous offspring: HH0, HH1, HH2, HH3, HH4, HH5, and HH6 in Holstein cattle. HH0 causes Brachyspina syndrome (HH0), which is a congenital inherited lethal defect in Holstein cattle that causes embryonic death, stillbirth and other deformities. (e.g., TY). HH1 is a nonsense mutation in the APAF1 gene. HH3 is caused by an SNP in the SMC2 gene causing a single amino acid substitution. HH4 is caused by genetic lesions in the GART gene. HH5 is caused by genetic lesions in the TFB1M gene. HH6 is caused by disruption of the ATG initiation codon in the SDE2 telomere maintenance homolog gene. The genetic defect associated with HH2 is still unknown.

Cattle suffer from a number of genetic diseases that are monogenic disorders inherited in a Mendelian fashion. Various genetic diseases are known in the art. See for example, Garrick and Ruvinsky, “The Genetics of Cattle,” 2^{nd} Edition, CAB International, Oxfordshire UK 2015; see also Parkinson et al., “Diseases of Cattle in Australasia,” ISBN 9780958363447 Jolly et al., “Genetic Diseases of Cattle,” Chapter 21, each hereby incorporated by reference in their entireties.

Several other health traits are tracked in Holstein cattle, including hypocalcemia (milk fever), Displaced Abomasum (DA), ketosis (KETO), mastitis (MAST), metritis (METR), and retained placenta (RETP). MFEV is a disorder in which lactating cattle secrete too much calcium in the early stages of lactation. DA is a disorder that causes the cow's abomasum (“true stomach”) to fill with gas and move from the floor of the cow's abdomen to one side of the rumen, a condition which requires veterinary intervention. KETO is a disease caused by a cow using more energy either in general or to make milk in particular than is present in their energy intake. MAST is an inflammation of the cow's udder, frequently caused by bacteria. METR is an inflammation of both the endometrium and muscular layers of a cow's uterus. RETP is when the cow's side of the fetal placenta fails to separate from the calf's side such that fetal membranes and other tissues haven't been expelled 24 hours after birth. Each of these traits is expressed in a percentage increase over the breed average resistance. (The average resistance is calculated by subtracting the incidence rate from 100%) For example, if a bull has a MFEV of +0.5, his daughters are expected to have a resistance to milk fever that is 0.5% higher than the breed average. Genomic and genetic evaluations are provided for Holstein animals; the evaluation of each trait is expressed in percentage points of resistance above or below the breed average.

### Porcine

In contrast with bovine breeding, porcine selection is less centralized. Each breeder uses their own custom index depending on their breeding goals. Many rely on the “best linear unbiased prediction” or BLUP statistical model. Most software for calculating the trait index is customized for a specific breeder, but publicly available programs are known in the art. There are several art recognized statistical models, which also include GBLUP, which brings genomic markers into the model (Stock, J., Front. Genet. 2020; 11: 568). These models are used to predict Estimated Breeding Values (EBVs; Published US application 2005/0221322 by Fox et al.). It is within the skill of the ordinary artisan to predict which animals will lead to offspring that fulfill the goals of the breeding program. As with bovines, methods of collecting gametes are known in the art.

### Embryo Creation

A large plurality of embryos is created via methods known in the art, such as in vitro fertilization (IVF) or artificial insemination (AI). AI embryos are collected using methods known in the art (for example flushing) and then blastocyst stage embryos are collected and advanced to the next step. IVF embryos can be observed and sent through to the next step of the process at the appropriate time.

### Embryonic Stem Cell Generation

Embryonic stem cells (ESCs) can be generated, for example, by dissecting out the inner cell mass (ICM), dissociating the cells, and then reseeding them onto a substrate. Alternatively, or in addition, whole embryos or blastocysts matured therefrom can be seeded onto a substrate to plate down to become embryonic stem cells. The zona pellucida can be removed via any method known in the art, including enzymatic digestion or laser assisted hatching.

### Inner Cell Mass Dissection

Inner cell masses can be isolated from blastocysts using a variety of techniques that are similar across species. ICM cells can be isolated using microsurgery, enzymatic digestion, immunosurgery, or mechanical separation.

Blastocysts can be placed in a suitable cell growth media such as HEPES-TL medium and then microsurgery can be performed via any technique known in the art, for example but without limitation using an ophthalmic scissors (see, for example Gao, X. et al., Nature Cell Biology, 2019, 21, 687-699) or using a microblade connected to micromanipulation equipment (for example, a NT88-V3 high precision micromanipulator from Nikon/Narishige) attached to an inverted microscope (for example, a TE2000-U from Nikon; see, for example Bogliotti, Y. S., et al., Proc. Natl. Acad. Sci. USA, 2018, 115, 2090-2095).

The zona pellucida of blastocysts can be removed by enzymatic digestion, such as via pronase, trypsin, or a combination thereof, until the trophoblasts begin to disperse in the microdrop. ICM cells can then be washed in stem cell culture medium and isolated with the aid of two fine needles and a pulled mouth micropipette (see, for example, Li M, et al., Mol. Reprod. Dev., 2003, 65, 429-434).

Alternately, the ICM can be isolated using immunosurgery techniques by incubating the blastocyst in Tyrode's Solution (available from commercial sources such as Sigma-Aldrich) or pronase solutions to remove zona, followed by antibody and complement system mediated lysis of the trophoectoderm (TE). Blastocysts can be incubated in stem cell culture medium supplemented with 10-20% anti-Porcine Serum (for pigs) or anti-Bovine Serum (for cattle) for 30 min to 1 hour. The serum can be antibodies raised against the required species from any suitable host, such as rabbit, goat, or rat. The serum can be washed out and then the cells can be incubated in stem cell culture medium supplemented with 10-20% complement serum for 30 min to 1 hour. Complement serum from various sources is commercially available, and sources include guinea pig complement. Once treated, ICMs can then be isolated from lysed trophoblast cells by pipetting and washed multiple times in stem cell culture medium (see, for example, Hou, D. R., Sci Rep. 2016, 6, 25838). For mechanical separation, zona-free blastocysts can be obtained by both spontaneous and artificial hatching processes and then washed three times. The ICMs can then be separated from the trophectoderm by gently pipetting with fine-pulled glass capillary pipettes in stem cell culture media (see, for example Jung, S. K., et al., J Vet Sci., 2014, 15, 519-528).

Alternatively, an embryonic stem cell culture can be initially established by plating a whole blastocyst. The zona pellucida can be removed via pronase or Tyrode's Solution (described supra) and then the entire blastocyst can be plated on feeder cells in media as described infra. The media can be formulated to promote ESC growth and not TE growth. So, the ESC will grow and the TE will have limited growth. Therefore, over time, the TE cells will be outcompeted and not be present in the culture.

Alternatively, the ICM can be isolated by laser assisted removal of the TE, which can be performed as follows: blastocysts can be secured by two holding pipettes with the ICM being positioned at 9 o'clock (relative to the field of view). Once adequate tension is established, approximately 10 infrared laser pulses (300 mW×1 ms, ZILOS-TK™, Hamilton Thorne Research, Beverly, MA) can be fired to split the blastocyst into two unequal portions—the smaller consisting of ICM, the larger consisting exclusively of trophoblast cells. The dissected ICM cells can be plated on feeder cells or directly into protein coated culture vessels.

### Cattle Embryonic Stem Cells

Once isolated, the isolated ICM cells are placed in a culture dish i) coated with an organic matrix such as fibronectin, Matrigel, or Vitronectin, ii) coated with decellularized extra cellular matrix (ECM; porcine gelatin, mixture of collagen IV, fibronectin, laminin and vitronectin) or iii) seeded with inactivated feeder cells. Suitable inactivated feeder cells include mitotically inactivated feeder cells such as gamma irradiated mouse embryonic fibroblast (MEF) or mitomycin treated MEF, human fetal muscle cells, human fetal fibroblasts, human adult fallopian tubal epithelial cells, human dermal fibroblasts, human amniotic mesenchymal cells, human amniotic epithelial cells, mouse bone marrow stromal cells, murine amniocytes, MEF SNL line, human amniocytes, human foreskin fibroblasts, human amniotic mesenchymal cells, pericellular matrix of decidua-derived mesenchymal cells, or another type of inactivated feeder cells. Inactivated MEF cells can be plated at 570,000 to 650,000 cells per well of a 6 well plate, 228,000 to 350,000 cells per well of a 12 well plate, or 100,000 to 118,000 cells per well of a 4 well plat. Plated MEF are good for 7 days. Cells are cultured in a basal media that is completely devoid of growth factors FGF2 and TGFβ.

Suitable media include CTFR Culture media (Ludwig, T. E., et al., Nature Methods, 2006, 637-646), ES culture media (Wu X, et al., Sci Rep. 2016 6, 28343), modified TeSR1 (e.g. lacking TGFP), TeSR1, N2B27, or E6 (available from Thermo-Fisher, catalog number A15164091). A modified N2B27 medium is described in Example 22. Briefly, this medium contains DMEM/F12 medium, Neurobasal Medium, 0.5×B-27 Supplement, 1×N2 supplement, 1×MEM Non-essential amino acid solution, 1× GLUTAMAX™ (Life Technologies Corporation, Carlsbad, CA), penicillin-streptomycin, and 0.1 mM 2-Mercaptoethanol. The media can be supplemented with an inhibitor of wingless-integrated signaling (“WNT signaling”) and bFGF, FGF2, FGF4, or SUN 11602. A variety of signaling molecules and small molecules are known in the art to inhibit WNT signaling and are commercially available from suppliers such as Sigma-Aldrich (St. Louis, MO) and R&D systems (Minneapolis, MN). Such molecules include iwr-1, iwr-1-endo, iwp-2, Box5, iCRT3, sclerostin, dkk2, dkk1, LF3, CCT036477, FH535, cardamonin, IWP-L6, Wnt-C59, niclosamide, XAV-939, ICG-001, LGK-974, CP21R7, NCB-0846, PNU-74654, salinomycin, KY021 11, WIKI4, PRI-724, KYA1797K, 2,4-diamino-quinazoline, Ant 1.4Br, Ant 1.40, apicularen, bafilomycin, ETC-159, G007-LK, G244-LM, IWR, NSC668036, PKF1 15-584, pyrvinium, Quercetin, Shizokaol D, BC2059, and a combination thereof. A preferred embodiment is N2B27 medium supplemented with 20 ng/mL FGF and 2.5 μM IWR.

After 6-7 days in culture, ICM outgrowth can be passaged. This usually comprises dissociating the cells enzymatically (e.g. with pronase, trypsin, or recombinant trypsin) or mechanically (as discussed supra) and reseeding them onto newly prepared feeder cells with either i) culture medium and a Rho kinase (ROCK) inhibitor or ii) culture medium, FBS, and a serum replacement. Serum replacements are well known in the art and generally contain supplementary amino acids, anti-oxidants, and proteins such as insulin, transferrin, and lipid rich albumin. Serum replacements may also contain trace elements. Commercially available serum replacements include N2, B27, and KNOCKOUT® Serum Replacement (KSR; Gibco, trademark is to Thermo Fisher Scientific, Waltham, MA). A full discussion of serum replacements can be found, for example, in published application US20020076747. Suitable ROCK inhibitors include Y-27632, AS1892802, Fasudil hydrochloride, GSK 269962, GSK 429286, H 1152, Glyclyl-H 1152, HA 1100, OXA 06, RKI 1447, SB 772077B, SR 3677, PD-325901, and TC-S 7001. Passaged cells can be seeded into new wells containing the same media and feeder cells. Once established, media can be changed daily. The cells can be split 1:10 to 1:20 every 4-5 days. Cells can be maintained at 37° C.-38.5° C. in a humidified CO₂ incubator.

### Porcine Embryonic Stem Cells

Isolated ICM cells can be cultured on mitotically inactivated and metabolically active feeder cells, such as STO or MEF cells. These feeder cells can be mitotically inactivated using gamma irradiation or mitomycin C treatment. Other exemplary feeder cells are described supra. The ICM cells can be cultured in 5% 02, 5% CO₂ and 90% N₂ at 37.0° C.-38.5° C. in a humidified atmosphere until outgrowths appear. (See exemplary procedures in Hou, D. R., Sci Rep. 2016, 6, 25838; Gao, X. et al., Nature Cell Biology, 2019, 21, 687-699; Li, M., et al., Mol. Reprod. Dev., 2003, 65, 429-434; Michalska, A. Curr. Protoc. Stem Cell Biol., 2007, Chapter 1:Unit1C.3). Several media can be used for these cultures, such as, but without limitation, pESC, pESLC, or pEPSCM.

pESC is a 1:1 mixture of low-glucose Dulbecco's Modified Eagle Medium (DMEM) (Invitrogen) and Ham's F-10 medium (Invitrogen). This mixture further comprises: 2 mM 1-glutamine, 1% (v/v) non-essential amino acids (Invitrogen), 0.1 mM β-mercaptoethanol (Invitrogen), and 1% (v/v) penicillin-streptomycin (Invitrogen), and 15% (v/v) FBS (HyClone) supplemented with 20 ng/mL recombinant human basic fibroblast growth factor (bFGF; Invitrogen), 20 ng/mL recombinant human stem cell factor (SCF; R&D Systems, USA), and 20 ng/mL recombinant human leukemia inhibitory factor (LIF) (Park, J. K., et al., PLoS One. 2013, 8, e52481; Jung, S. K., et al., J Vet Sci., 2014, 15, 519-528; Yuan, Y., et al. Cell Death Discov., 2019, 5, 104).

Another published porcine medium comprises DMEM supplemented with 0.1 mM b-mercaptoethanol (Amresco; 3151B55), 100 IU/ml penicillin, 0.05 mg/ml streptomycin, 0.1 mM MEM nonessential amino acids (Gibco; 08540), 20 ng/ml recombinant human-fibroblast growth factor-basic (rh-bFGF, Sigma; F0291), 40 ng/ml recombinant human-leukemin inhibitory factor (rh-LIF; Sigma; L-5283), nucleosides (0.03 mM adenosine, 0.03 mM guanosine, 0.03 mM cytidine, 0.03 mM uridine, and 0.01 mM thymidine; Sigma), and 16% FBS. (Li, M., et al., Mol. Reprod. Dev., 2003, 65, 429-434).

Porcine Expanded Potential Stem Cells Medium (pEPSCM) comprises 500 ml N2B27 basal media: 0.2 μM CHIR99021 (a GSK3 inhibitor and WNT agonist; Tocris, cat. no. 4423), 0.3 μM WH-4-023 (an SRC inhibitor; Tocris, cat. no. 5413), 2.5 μM XAV939 (a tankyrase inhibitor; Sigma cat. no. X3004) or 2.0 μM IWR-1 (a wnt inhibitor; Tocris, cat. no. 3532), 65.0 pg/ml vitamin C (Sigma, cat. no. 49752-100G), 10.0 ng/ml LIF (Stem Cell Institute (SCI), University of Cambridge), 20.0 ng/ml Activin (SCI) and 0.3% FBS (Gibco, cat. no. 10270)+10 μM Y27632 (ROCK inhibitor) (Gao, X. et al., Nature Cell Biology, 2019, 21, 687-699).

A modified N2B27 medium is described in Example 22 which is also suitable for preparing porcine embryonic stem cells.

Media suitable for preparing and maintaining porcine embryonic stem cells can comprise a GSK3 inhibitor, a tankyrase inhibitor, or a combination thereof. Skilled artisans will recognize that an effective amount of a different GSK3 inhibitor, such as but without limitation, commercially available inhibitors AR-A 014418, A 1070722, SB 415286, TCS 2002, 3F8, TDZD 8, TC-G 24, BIO-acetoxime, indirubin-3′-oxime, TWS 119, TCS 21311, SB 216763, BIO, lithium carbonate, kenpaullone, alsterpaullone, and CHIR 98014. (All of these small molecules are available to purchase from R&D systems). Further, alternate tankyrases inhibitors can include AZ 6102, JW 55, TC-E 5001, and WIKI4. Suitable media may also comprise a ROCK inhibitor and or an inducer of bFGF signaling. A preferred embodiment is N2B27 medium as described in Example 22 and supplemented with 10 ng/ml bFGF, 3 μM CHIR99021, and 1 μM PD0325901. The PD0325901 can be withdrawn after 6 days.

The culture medium can be changed after every 1-2 days. In general, cells form colonies after 5-8 days of culture. Then embryo derived colony forming cells can be mechanically isolated using a pulled glass pipette and reseeded onto fresh feeder cells in stem cell culture media. Alternatively, the cells can be removed using a trypsinization process. Cells generally form well defined colonies after 3-4 days and can be routinely passaged via the pulled glass pipette method or trypsinization every 5-7 days. The cells can be cultured in humidified conditions with 5% 02, 5% CO₂ and 90% N₂ at 37-38.5° C. with daily media changes.

Stem cells can be validated using alkaline phosphatase assay, ESC specific mRNA and protein-based markers analysis, and/or an embryoid body formation, spontaneous differentiation, and teratoma formation assay (see Park, J. K., et al., PLoS One. 2013, 8, e52481; Jung, S. K., et al., J Vet Sci., 2014, 15, 519-528; Yuan, Y., et al. Cell Death Discov., 2019, 5, 104 for exemplary methods).

Established ESCs can also be evaluated to determine their status as naïve (true ESCs) or primed based on low or no expression of mRNA (MHC I antigen, XIST) and protein (H3K27me3) based marker in naïve ESC compared to primed ESC (see Zhang, M., et al., FASEB J., 2019, 33, 9350-9361).

### Genotyping of ESCs

Aliquots of each stem cell line can be taken for use in genotyping. In some embodiments, embryos may be cultured while the genetic tests are being performed on a portion of the cells from the same embryos. If the test results are positive, the cultured embryos may be transferred to a recipient for production of offspring. Alternatively, cells from these positive embryos may be used in a nuclear transfer process, or any other process known in the art, to produce genetically superior animals. In various embodiments, methods are disclosed for expanding and freezing of embryonic cells or embryonic stem cells. Genetic tests may be performed on a portion of the expanded cells. If the test results are favorable, the remaining cells from the same embryos may be thawed and used to produce offspring. In another embodiment, biopsy sample(s) may be obtained from embryos that have been cultured for about 3-7 days after fertilization. General methods of embryo biopsy are known in the art and disclosed, for example, in Polisseni et al., Fertility and Sterility, 2010, 93, 783-788 and Lopes et al. Theriogenology, 2001, 56, 1383-1392. The biopsied cells may be cultured individually to expand the cells for about 2-10 days, or even longer. The expanded cells from the biopsy sample may be subject to genetic test, such as whole genome analysis (WGA) or genotyping, while the rest of the embryos are cultured. Multiple displacement amplification (MDA) is one type of WGA technique, which may be used to increase the amount of DNA from biopsies for analysis (see. e.g., Lauri et al. Genomics, 2013, 101, 24-29).

### Bovine Genotyping

There are several methods of genotyping bovine animals or bovine cells known in the art. The most common is the 50 k Bovine SNP chip. The genotype of ESCs can be determined using the ILLUMINA® Infinium HTS BeadChip (ILLUMINA®, Inc., San Diego, CA) microarray system using the BovineSNP50 v3 BeadChip microarray kit (BeadChip) according to manufacturer's instructions. The BeadChip contains 53,714 highly informative SNP probes uniformly distributed across the entire genome of major cattle breed types at a median spacing of 37.4 kb. The SNP probes are validated in 18 common beef and dairy breeds. The BeadChip is a collaboration of ILLUMINA®, Inc., the USDA-ARS, the University of Missouri, and the University of Alberta. More than 22,000 SNP probes target novel SNP loci found in pooled populations of economically important beef and dairy cattle. The BeadChip is well known and extensively used by those of skill in the art. Other suitable genotyping chips and technologies can be used, including but not limited to the GENESEEK® GENOMIC PROFILER™ High-Density array (GGP HD150K; 139376 SNPs; Neogen Genomics, Lincoln NE), the NEOGEN® GGP Bovine 50 k chip (47843 SNPs; Neogen Genomics, Lincoln NE), and the NEOGEN® GGP Bovine 100 k chip (100,000 SNPs).

A wide variety of publicly available resources are known in the art including the bovine reference genome (Bovine Genome Sequencing and Analysis Consortium, Science 324(5926):522-528), Btau (available at ftp.hgsc.bcm.tmc.edu/), and the Bovine HapMap Consortium data set (Bovine HapMap Consortium, Science 324(5926):528-32 (2009) available on the internet at bovinehapmap.org). Analysis tools and sequence data are maintained by the Bovine Genome Database (BGD) that is supported by the European Union's Seventh Framework Programme for research, technological development, and demonstration (Grant Agreement No. 613689), and the USDA National Institute of Food and Agriculture. BGD is hosted at the University of Missouri.

### Porcine Genotyping

Like bovine genotyping, there are several methods of porcine genotyping known in the art. ILLUMINA® sells the PorcineSNP60v2 BeadChip, which contains approximately 64,000 SNP probes. In order to provide for different breeder goals and indices, this chip also provides space for an additional 25,000 user supplied SNP probes. This chip was designed in collaboration with the International Porcine SNP Chip Consortium, comprising researchers from various governmental and academic institutions. This chip is designed to enable marker assisted swine selection as discussed supra. Other suitable genotyping chips and technologies can be used, including but not limited to the GENESEEK® GENOMIC PROFILER™ (GGP Porcine 80K; Neogen Genomics, Lincoln NE) and the NEOGEN® GGP Porcine 50 k chip (Neogen Genomics, Lincoln NE) that contain ˜80,000 and 51,000 SNPs respectively. Other genotyping chips and technologies are commercially available and known in the art.

Once genotyped, the ESC lines with the most desirable traits are selected in a similar manner to the selection step for both porcine and bovine breeding. Especially desirable ESC lines can be used to generate animals using, for example, nuclear transfer methods such as those described in Ross and Cibelli, Methods Mol. Biol., 2010, 636, 155-177. See also U.S. Pat. No. 6,011,197 issued Jan. 4, 2000, to Strelchenko et al.

Alternatively, or in addition, ESC lines can be selected for in vitro gametogenesis as discussed infra.

### Gametogenesis

Embryonic stem cell lines are pluripotent and can be induced to form many different cell types, including gametes. The type of gametes produced during gametogenesis is dependent on the genotype of the cells—XY bearing ESC lines can be induced to create spermatozoa or spermatid-like cells and XX bearing ESC lines can be induced to create oocytes or oocyte-like cells.

ESC Differentiation into Primordial Germ Cell-Like Cells (PGCLCs)

Thus far, primordial germ cell-like cells have been induced from embryonic stem cells via an intermediate cell type. These progenitor cells are various types of pluripotent cells such as epiblast-like cells, formative cell-like cells, mesoderm-like cells, or extraembryonic endoderm (Xen) cells. Any pluripotent cell type that can differentiate into a primordial germ cell-like cell can be used as a progenitor cell.

Also contemplated is making PGCLCs directly from embryonic stem cells.

### Bovine

Embryonic stem cells can be enzymatically dissociated (such as in trypsin) and cultured feeder free on a dish precoated with extracellular matrix or basal lamina molecules. Suitable molecules include collagen IV, poly-L-omithine, and laminin, gelatin, vitronectin, or fibronectin. For example, 16.7 μg/ml of human plasma fibronectin (Millipore) in PBS can be placed in a 12 well plate and incubated at 37° C.-38.5° C. (See other exemplary procedures in Wang H, et al., Sci Rep., 2016, 6, 27256; Hayashi, K., et al., 2011, Cell, 146, 519-532; Ciccarelli M, et al., Proc Natl Acad Sci USA, 2020, 117, 24195-24204).

These cultured ESCs can then be induced into progenitor cells such as epiblast-like cells (EpiLCs), formative-like cells, or mesoderm-like cells. An exemplary method to make EpiLCs is to culture ESCs in serum free medium comprising an activin signaling inducer, a bFGF signaling inducer, an insulin signaling inducer, or a combination thereof. Inducers of each type of signaling are known in the art and discussed infra. Alternatively or in addition, cultured bESCs can be induced into bovine formative-like cells in serum free medium comprising a bFGF signaling inducer, an activin A signaling inducer, a WNT signaling inducer/GSK3 inhibitor, or a combination thereof. Alternatively or in addition, bESCs can be can be induced into mesoderm-like cells in serum free medium comprising an activin A signaling inducer and a wnt signaling inducer/GSK3 inhibitor. Further, the serum free medium can comprise a serum replacement. Exemplary serum free media include N2B27 media and GK15 media, described in detail in Example 22 and Example 33, respectively. Both bovine formative cells and bovine mesoderm-like cells can be induced feeder free on fibronectin coated plates. Exemplary activin signaling inducers include activin A, BMP4, nodal, alantolactone, stauprimide ([9S-(9α,10β,11β,13α)]-N-(2,3,10,11,12,13-Hexahydro-10-methoxy-9-methyl-1,3-dioxo-9,13-epoxy-1H,9H-diindolo[1,2,3-gh:3′,2′,1′-lm]pyrrolo[3,4-j][1,7]benzodiazonin-11-yl)-N-methylbenzamide), SB-431542 (4-[4-(1,3-benzodioxol-5-yl)-5-(2-pyridinyl)-1H-imidazol-2-yl]benzamide), A01([4-[[4-Chloro-3-(trifluoromethyl)phenyl]sulfonyl]-1-piperazinyl][4-(5-methyl-1H-pyrazol-1-yl)phenyl]methanone), alantolactone, GDF8, and conophylline (dimethyl (2R,6R,11R,13S,14S,23S,24S,25S,36R,39R,40R)-14,25-diethyl-24,33-dihydroxy-31,32-dimethoxy-12,22-dioxa-1,9,18,29-tetrazadodecacyclo[23.13.1.16,9.02,23.03,21.05,19.06,17.011,13.028,36.030,35.036,39.014,40]tetraconta-3,5(19),16,20,27,30,32,34-octaene-16,27-dicarboxylate). Exemplary inducers of bFGF signaling include bFGF, SUN 11602 (4-[[4-[[2-[(4-Amino-2,3,5,6-tetramethylphenyl)amino]acetyl]methylamino]-1-piperidinyl]methyl]benzamide), sucralfate (CAS 54182-58-0), and FGF-P (a peptide mimetic published in Casey-Sawicki, K., et al., Health Phys 2014, 106, 704-12). Exemplary inducers of insulin signaling include insulin, zinc chloride, zinc nitrate, zinc bromide, zinc sulfate zinc sulfate-7 water, bpV (CAS 42494-73-5), BMOV (CAS 38213-69-3), vanadyl rosiglitazone, vanadyl trehalose, vanadyl metformin, vanadyl quercetin (the vanadyl molecules are all published in Jiang, P, Applied Biochemistry and Biotechnology, 2016, 180, 841-851), demethylasterriquinone B1 (2-[2-(1,1-Dimethyl-2-propenyl)-1H-indol-3-yl]-3,6-dihydroxy-5-[7-(3-methyl-2-butenyl)-1H-indol-3-yl]-2,5-cyclohexadiene-1,4-dione), and BRD 7552 (methyl [2,3-O-bis(Benzo[1,3]dioxol-5-yl-carbamoyl)]-4-O-(4-ethoxycarbonyl-phenylcarbamoyl)-α-D-glucopyranoside). Other inducers of insulin signaling can include media additives containing insulin such as a serum replacement.

There are several methods of inducing PGCLCs from progenitor cells. A floating culture of EpiLCs can be plated in a well of a low-cell-binding U-bottom 96-well plate in a serum-free medium comprising an activator of insulin signaling, at least one inducer of BMP signaling, an inducer of LIF signaling, an inducer of SCF signaling, an inducer of EGF signaling, an inducer of WNT signaling, or a combination thereof. Exemplary serum free mediums include commercially available DMEM, MEMalpha, DMEM/F12, Ham's F-10, Ham's F-12, RPMI 1640, Opti-MEM, RPMI 1640, Opti-MEM, and GMEM. Serum free media N2B27 and GK15 media are described in Example 22 and Example 33, respectively. Exemplary inducers of insulin signaling are listed supra. Exemplary inducers of BMP signaling can include BMP4, BMP8b, BMP7, TGFβ, sb4 (2-[[(4-Bromophenyl)methyl]thio]benzoxazole), triamcinolone, isoliquiritigenin and, 4′-hydroxychalcon (Vrijens, K, et al., PLOS One, 2013, 8, e59045), C1=C(CC(C)C)O═NC1C(═O)Nc1:c:c(Cl):c(F):c:c:1, C(OC1=CC(CC)=CC═C1)(=O)Nc1:c:c(N(O)=O):c(F):c:c:1, C1C═SC(C(OCC(═O)Nc2:c:c:c(OC(F)F):c:c:2)=O)C=1C (these fluorinated compounds are described in Genthe, J. R., ACS Chem Biol. 2017 Sep. 15; 12(9): 2436-2447), km11073 (WO2019042889), PD07824, SVAK-12 (Kato, S., et al., Mol Cell Biochem., 2011, 349, 97-106), C1=C(C(F)(F)F)C(Cl)=CC═C1S(O)(O)N1CCN(C(═O)C2=CC═C(N3C(C)=CC═N3)C=C2)C C1, or 1-Ethyl-6-fluoro-4-oxo-7-[4-(3-phenylpropanoyl)-1-piperazinyl]-1,4-dihydro-3-quinolinecarboxylic acid. The inducer of LIF signaling can be LIF. The inducer of SCF signaling can be SCF. Exemplary inducers of EGF signaling include EGF, amphiregulin, EPR, and HB-EGF. Exemplary inducers of WNT signaling include WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I (2-Amino-4-(3,4-(methylenedioxy)benzylamino)-6-(3-methoxyphenyl)pyrimidine, CAS 853220-52-7), Wnt Agonist II (5-(Furan-2-yl)-N-(3-(1H-imidazol-1-yl)propyl)-1,2-oxazole-3-carboxamide), CHIR99021, LP 922056, BML 284, WAY 316606 hydrochloride, SGC AAK1 1, CHIR 98014, and Foxy 5.

Alternatively, the floating culture of progenitor cells can be induced in a serum-free medium comprising an activator of insulin signaling, at least one inducer of BMP signaling, an inducer of LIF signaling, an inducer of SCF signaling, an inducer of EGF signaling, or a combination thereof. (Exemplary inducers and media are discussed supra.)

The cells can be maintained at 37° C.-38.5° C. at 5% CO₂ (Wang H, et al., Sci Rep., 2016, 6, 27256; Hayashi, K., et al., 2011, Cell, 146, 519-532).

Alternatively, progenitor cells can be seeded on mouse embryonic fibroblast (MEF) feeder layer in basal culture medium. The basal medium can further comprise at least one inducer of BMP signaling, an inducer of SCF signaling, at least one inducer of insulin signaling, or a combination thereof. Exemplary basal culture mediums include NEUROBASAL™ (Gibco, Waltham, Massachusetts, USA) and DMF/12 medium (Hyclone, Logan, Utah, USA) (1:1), supplemented with 100× N-2 Supplement (Gibco) and 50×B-27™ Supplement (Gibco)—called N2B27 media. Exemplary signal inducers are described supra.

Alternatively, bovine formative cells and/or bovine mesoderm-like cells can be induced in feeder free culture using GK15 base medium supplemented with an inducer of LIF signaling, an inducer of SCF signaling, an inducer of EGF signaling, and an inducer of BMP signaling. (Exemplary signaling inducers are discussed supra.) Further, GK15 medium can contain serum replacement comprising an inducer of insulin signaling. (GK15 medium is described in Example 33.)

In a final alternative, ESC can be differentiated into PGCLC by using CRISPR mediated multiplex activation of endogenous transcription factors such as PRDM1, PRDM14, STELLA, SOX17, and/or SOX2 to induce the expression of GDF9, VASA, DAZL, NANOS2, and NANOS3. These markers are known to play a role in PGC differentiation (Wang H, et al., Sci Rep., 2016, 6, 27256; Wang G, et al., Nat Immunol., 2019, 20, 1494-1505; Ideta, A., Sci Rep., 2016, 6, 24983; Ciccarelli, M., Proc. Natl. Acad. Sci. USA, 2020, 117, 24195-24204). In some embodiments, the CRISPR mediated activators of endogenous transcription factors can be bred out of animals according to standard methods. In alternative embodiments, the CRISPR mediated activators can be transiently transfected in the culture cells so that they are not present in the resulting animals.

In order to remove undifferentiated ESCs, the resulting PGCLC cultures can be sorted by fluorescence assisted cell sorting (FACS) using PGC specific cell surface markers including Sda/GM2-glycan (Klisch, K., Reproduction, 2011, 142, 667-674), α-SSEA-1 (Fox N, Dev. Biol., 1981, 83, 391-398), αSSEA-3, SSEA-4 (Shevinsky, L. H., et al., Cell, 1982, 30, 697-705), EMA-1 (Hahnel, A. C. and Eddy, E. M., Gamete Res., 1986, 15, 1235-1244), TG-1 (Donovan P J, et al., J. Cell Sci., 1987, 8, 359-367), c-kit (De Miguel, M. P., Proc. Natl. Acad. Sci. USA, 2002, 99, 10458-10463), or TNAP (MacGregor, G. R., et al., Development, 1995, 121, 1487-1489).

Functional and genetic identity can be validated by analyzing the transcriptomic signature compared to bovine PGC in vivo and purity of PGCs can be determined by alkaline phosphatase (APase) staining.

PGCLC can be maintained in culture in the absence of somatic cells using a method described by Farini et al., Dev. Biol., 2005, 285, 49-56. or in the presence of somatic cells of male or female embryonic gonads (Hayashi, K. and Saitou, M., Nature Protocols, 2013, 1513-1524).

### Porcine

Porcine ESCs (pESCs) can be maintained on feeder cells. In order to prepare the cells for differentiation they can be adapted and maintained feeder free on a dish coated with basal laminin molecules. Suitable molecules include vitronectin, fibronectin, poly-L-omithine (0.01%; Sigma)+laminin (10 ng/ml; BD Biosciences) or gelatin. The cells can be maintained on a basal medium such as N2B27. N2B27 is a 1:1 mixture of DMEM/F12 (Gibco, 11320-033) supplemented with N2 (Gibco, 17502-048) and Neurobasal medium (Gibco, 21103-049) supplemented with B27 (Gibco, 17504-044), 25 μg/ml insulin and 50 μg/ml BSA. The basal medium can be further supplemented with activin A (20 ng/ml, PeproTech), bFGF (12 ng/ml, Invitrogen) and 1% KSR (Gibco) to maintain the cells' pluripotent status. Alternatively, they can be maintained on N2B27 medium as prepared in Example 22 supplemented with 10 ng/ml FGF and 3 μM CHIR99021. The medium can be changed every day.

For PGCLC differentiation, ESC can be induced into progenitor cells. The embryonic cells can be digested into single cells and then treated with at least one inducer of BMP signaling, an inducer of SCF signaling, an inducer of EGF signaling, an inducer of LIF signaling, or a combination thereof in serum-free medium. Alternatively, the cells can be treated with an inducer of activin signaling, an inducer of WNT signaling, or a combination thereof. Alternatively, the cells can be treated with an inducer of bFGF signaling, an inducer of activin signaling, an inducer of WNT signaling, or a combination thereof. Exemplary signaling molecules are listed supra. An exemplary medium can be GMEM supplemented with 15% KSR, NEAA, L-glutamine, penicillin/streptomycin, and β-mercaptoethanol as described in Wang, H, et al., Sci Rep., 2016, 6, 27256. Other exemplary media include N2B27 and GK15 media, described supra.

Alternatively, ESC can be differentiated into PGC by using CRISPR mediated multiplex activation of endogenous transcription factors, specifically a combination of PRDM1, PRDM14, STELLA, SOX17, OCT4, and SOX2. These factors are known to play a role in PGC differentiation (see, for example, Wang, H, et al., Sci Rep., 2016, 6, 27256; Wang G, et al., Nat Immunol., 2019, 20, 1494-1505). In some embodiments, the CRISPR mediated multiplex factors can be bred out of the animals using standard methods. Alternatively, the CRISPR mediated multiplex factors can be transiently transfected in the induced culture cells.

In order to remove undifferentiated ESCs, the resulting PGCLC can be sorted by FACS using PGC specific cell surface markers including Sda/GM2-glycan (Klisch, K., et al., Reproduction, 2011, 142, 667-674), a-SSEA-1 (Fox, N., et al., Dev Biol, 1981, 83, 391-398), αSSEA-3, SSEA-4 (Shevinsky, L. H., et al., Cell, 1982, 30, 697-705), EMA-1 (Hahnel, A. C. and Eddy, E. M., Gamete Res., 1986, 15, 1235-1244), TG-1 (Donovan P. J., et al., J. Cell Sci., 1987, 8, 359-367), c-kit (De Miguel, M. P., et al., Proc. Natl. Acad. Sci. USA, 2002, 99, 10458-10463), or TNAP (MacGregor, G. R., et al., Development, 1995, 121, 1487-1489).

Alternatively, the progenitor cells can be induced with culture media comprising at least one inducer of BMP signaling, an inducer of LIF signaling, an inducer of SCF signaling, an inducer of epidermal growth factor (EGF) signaling, or a combination thereof. Exemplary inducers are discussed supra. At day 6, these cells may become PGCLCs.

Additionally, ESCs can be stimulated to become PGCLCs by causing the ESCs to express transcription factors such as NANOG, BLIMP1, TFAP2C, and SOX17. These transcription factors can be expressed by transfecting vectors comprising expression cassettes to transiently express the transcription factors, or by transfecting vectors comprising PIGGYBAC transposons comprising the transcription factors on inducible promoters. The culture medium can comprise an inducer of BMP signaling, an inducer of LIF signaling, an inducer of SCF signaling, an inducer of EGF, and a ROCK inhibitor. Suitable inducers and ROCK inhibitors are discussed supra. Uptake of transcription factors into the nucleus can be enhanced by the addition of dexamethasone.

### Spermatogenesis

In order to induce differentiation of spermatogonial stem cell-like cells (SSCLC), male genotype bovine or porcine PGCLCs can be dissociated and plated in medium comprising an inducer of retinoic acid signaling, an inducer of GDNF signaling, an inducer of testosterone signaling, or a combination thereof. Exemplary inducers of retinoic acid signaling include retinoic acid, retinoic acid p-hydroxyanilide, 9-cis retinoic acid, BMP4, TTNPB, methoprene acid, EC23, 1-Methyl-2-oxindole, adapalene, CD437, AC261066, CD1530, Ch 55, AM 580, AM 80, AC 55649, BMS 961, BMS 753, CD 2314, fenretinide, adapalene, EC 19, and SR 1078. Exemplary inducers of GDNF signaling include GDNF, Neurturin (NRTN), Artemin (ARTN), Persephin (PSPN), XIB4035, BT13 (N,N-diethyl-3-[4-[4-fluoro-2-(trifluoromethyl)benzoyl]piperazin-1-yl]-4-methoxybenzenesulfonamide), BT18 ([4-[5-[(6,7-dimethoxy-3,4-dihydro-1H-isoquinolin-2-yl)sulfonyl]-2-methoxyphenyl]piperazin-1-yl]-[4-fluoro-2-(trifluoromethyl)phenyl]methanone), BT44, and C12NC(C3=CC═C(C4=NC5C=C(C(O)=O)C═CC=5N4)C=C3)=NC1=CC═C(C(═O)O)C=2 (Ivanova, L., et al., ACS Omega 2018, 3, 1, 1022-1030). Exemplary inducers of testosterone signaling include testosterone, methyltestosterone, fluoxymesterone, oxymetholone, oxandrolone, ethylestrenol, stanozolol, danazol, methandrostenolone, norethandrolone, GDNF, dihydrotestosterone, and icariin. The medium can be changed every day (Wang, H, et al., Sci Rep., 2016, 6, 27256).

Alternatively, bovine or porcine PGCLCs can be differentiated in reconstituted testes. Porcine or bovine embryonic testis somatic cells can be prepared by isolating embryonic testis and dispersing the cells, for example with a collagenase. The cells can then be dissociated by treating them with trypsin. A single cell suspension can be obtained by filtration through a cell strainer, and cells can be collected by centrifugation. Cells obtained from wild type embryos can be depleted of PGC by magnetic cell sorting (MACS) using a PGC specific marker antibody conjugated with magnetic beads. Alternatively, somatic cells can be isolated from an embryo comprising a germ cell specific gene knockout animal (e.g NANOS2 knockout)—if so, the MACS sorting can be omitted.

These cells can then be mixed with allogenic PGCLCs in a 1:1 ratio and cultured in a-MEM supplemented with an inducer of insulin signaling, an inducer of BMP signaling, an inducer of retinoic acid signaling, an inducer of activin A signaling, or a combination thereof. Examples of signal inducers are described supra. The expression of meiosis specific markers such as RecA and Stra8 and spermatogonia stem cells (SSC) specific markers can be monitored. Cells can be confirmed as spermatogonia stem cell-like cells (SSCLC) when these markers are upregulated compared to day 0 PGCLC in culture to confirm PGCLC differentiation to SSCLC. (See Zhou, Q., et al., Cell Stem Cell, 2016, 18, 330-340).

Alternatively, PGCLCs can be reseeded onto a feeder layer in a basal culture medium. For example, the basal medium can be N2B27. The basal medium can further comprise an inducer of BMP signaling, an inducer of FGF signaling, an inducer of GDNF signaling, an inducer of LIF signaling, an inducer of insulin signaling, or a combination thereof. The cells can then be cultured until spermatogonial stem cell specific markers are upregulated compared to day 0 as assayed by methods known in the art to confirm a spermatogonial stem cell-like cell (SSCLC) state. The efficiency of this induction can be enhanced using Eif2s3y as a CRISPR associated gene. In some embodiments, the Eif2s3y expression cassette can be bred out of any resulting production animals.

For stable SSCLC maintenance and proliferation, SSCLC can be cultured in stem cell culture medium such as StemPro-34 serum free medium (SFM) (Invitrogen) supplemented with StemPro supplement (Invitrogen), 25 mg/ml insulin, 100 mg/ml transferrin, 60 mM putrescine, 30 nM sodium selenite, 6 mg/ml D-(1)-glucose, 30 mg/ml pyruvic acid, 1 ml/ml DL-lactic acid (Sigma), 5 mg/ml bovine albumin ImmunO (MP Biochemicals LLC, Solon, OH, USA), 2 mM L-glutamine, 50 μM 2-mercaptoethanol, minimal essential medium (MEM), vitamin solution (Invitrogen), MEM nonessential amino acid solution (Invitrogen), 100 μM ascorbic acid, 10 mg/ml D-biotin, 30 ng/ml b-estradiol, 60 ng/ml progesterone (Sigma), and 0.1% FCS. The media can further comprise 20 ng/ml EGF, 10 ng/ml FGF2, 100 ng/ml LIF, and 40 ng/ml GDNF (see Aponte et al., Reproduction, 2008, 136, 543-5572).

Next, the SSCLC can be differentiated into haploid spermatid-like cells (SLC). SSCLC can be cultured in a minimal media containing an inducer of insulin signaling, an inducer of testosterone signaling, an inducer of FSH signaling, Bovine Pituitary Extract, or a combination thereof. Examples of inducers of insulin signaling and testosterone signaling are discussed supra. Exemplary inducers of FSH signaling include FSH, corifollitropin alfa, FSHβ-CTP-α, N2-α FSH, pregnant mare's serum gonadotropin, thiazolidinone, and hexahydroquinoline. Bovine pituitary extract is commercially available, for example from Thermofisher, Sigma-Aldrich (Millipore Sigma), Lonza, and other biochemical manufacturers. The medium can be changed every 2 days. Cells can be cultured in 5% CO₂ at 37° C.-38.5° C. until haploid spermatid markers such as TP1, PRM1, acrosin, and haprin are upregulated. Upregulation can be measured via Western blot of samples taken from the culture as a whole.

Alternatively, SSCLC can be reseeded on a feeder layer in a 6-well-plate in basal culture medium, for example N2B27, comprising an inducer of retinoic acid signaling, an inducer of insulin signaling, or a combination thereof until haploid spermatid markers such as TP1, PRM1, acrosin, and haprin are upregulated.

The efficiency of this induction can be enhanced using Eif2s3y as a CRISPR associated gene. In some embodiments, the Eif3s3y gene can be bred out of the lines for production animals.

This induction produces round, haploid cells with spermatid markers. Spermatids can be used to fertilize oocytes through intracytoplasmic sperm injection (ICSI) via methods known in the art. Spermatozoa, if required, can be produced by incubating the SSLCs in suspension with gonad cells or media conditioned from gonad cells.

### Oogenesis

### Reconstituted Ovaries

FACS sorted porcine or bovine female PGCLC can be mixed with allogenic gonadal somatic cells in a 1:1 ratio in a minimal culture media supplemented with an inducer of insulin signaling and an inducer of retinoic acid signaling to form reconstituted ovaries. The gonadal somatic cells can be prepared by isolating embryonic ovaries and dispersing them with collagenase, followed by digesting the cells in trypsin. A single cell suspension can be obtained by filtration through a cell strainer, and cells are collected by centrifugation. Cells are then depleted of primordial germ cells (PGC) by magnetic cell sorting (MACS) using a PGC specific protein marker antibody conjugated with magnetic beads. Alternatively, this PGC depletion step can be obviated by using ovaries isolated from embryos with a female germ cell specific knockout (for example NANOS3 knockout animals).

Reconstituted ovaries can be placed on collagen coated membranes soaked in an in vitro differentiation (IVDi) medium. The IVDi medium can comprise: a minimal media supplemented with FCS, ascorbic acid, glutamine supplement, penicillin/streptomycin, and 2-mercaptoethanol. At 2-7 days of culture, the culture medium can be changed to IVDi medium comprising serum free medium supplemented with FCS, ascorbic acid, a glutamine supplement, penicillin/streptomycin, and 2-mercaptoethanol. From 5 days to 10 days of culture an estrogen receptor agonist can be added to the IVDi medium. Exemplary estrogen receptor agonists include ICI182780, ferutinin, estropipate, estrone-3-sulfate, S-equol, WAY-200070, DY 131, GSK 4716, AB-1, ERB-041, biochanin A, β-Estradiol, α-Estradiol, PPT, DPN, FERb 033, or AC 186. At 15-25 days of culture, individual secondary follicle like structures (2FLs) can be manually dissociated from the reconstituted ovaries (see below).

Electrically sharpened tungsten needles can be used for the isolation of individual secondary follicle like structure (2FLs). Interstitial cells between 2FLs can be removed. The 2FLs can be separated from the reconstituted ovaries (rOvaries) and placed at largely regular intervals on collagen coated membranes.

The single 2FLs on the collagen membranes can be soaked in IVG medium. The IVG medium can comprise: a minimal medium supplemented with FCS, a stabilizing polymer such as polyvinylpyrrolidone, ascorbic acid, a glutamine supplement, penicillin/streptomycin, M 2-mercaptoethanol, sodium pyruvate (Nacalai Tesque), a follicle-stimulating hormone (FSH) signal inducer, a TGFβ signal inducer, or a combination thereof. Exemplary FSH and BMP signal inducers are discussed supra. Exemplary TGFβ signal inducers include GDF9, BMP15 (which is also called GDF9b), and the exemplary BMP signaling inducers discussed supra. At 1-5 days of culture, the TGFβ signal inducers can be withdrawn from the medium and then follicles can be incubated in collagenase. After washing with minimal media supplemented with serum, the follicles can be cultured in IVG without TGFβ signal inducers. At 7-14 days of culture, cumulus-oocyte complexes (COCs) grown on the membrane can be picked up and transferred to IVM medium: minimal media supplemented with FCS, sodium pyruvate, penicillin/streptomycin can be supplemented with signaling molecules. The signaling molecules can be an FSH signaling inducer, an EGF signaling inducer, a gonadotropin signaling inducer, or a combination thereof. Exemplary FSH and EGF signaling inducers are discussed supra. Exemplary gonadotropin signaling inducers include hCG, chorionic gonadotropin, choriogonadotropin alfa, and corifollitropin alfa. At 16 h of culture, swollen cumulus cells are stripped from the oocytes by treating with hyaluronidase (Sigma), and then MII oocytes can be determined by 1st polar body extrusion.

### Directly Induced Oocyte-Like Cells

Vectors that allow for expression of the FIGLA, SOHLH1, LHX8, NOBOX, STAT3, TBPL2, DYNLL1, or SUB1 genes, or a combination thereof can be co-transfected into ESCs by using Lipofectamine 2000 (Thermo), nucleofection (Lonza Nucleofector Kit, VPH-1001, VPH-5012, or other) or electroporation (BTX). Suitable vectors include PiggyBAC based vectors with shield1 inducible promoters and vectors suitable for transient transfection. Various transient transfection systems are known in the art and are commercially available. After 24 h, the ES cells can be cultured with antibiotics. After 2-5 days, ES cells can be re-spread onto feeder cells. (Exemplary feeder cells are discussed supra.) At 3-7 days of culture, single colonies are picked up and seeded on feeder cells. To remove feeder cells, ES cells can be passaged under feeder-free conditions at least three times.

For the long-term induction of directly induced oocyte-like cells (DIOLs) without somatic cells, 50,000 ES cells can be transferred into the low-cell-binding U-bottom 96-well plate in basal medium that can be supplemented with FCS, ascorbic acid, a glutamine supplement, antibiotics, and 2-mercaptoethanol. The media can further comprise an SCF signaling inducer, a ROCK inhibitor, or a combination thereof. (Exemplary SCF signaling inducers and ROCK inhibitors are discussed supra.) At 2 days of culture, aggregates can be placed on TRANSWELL®-COL® membranes (CORNING®, CORNING®, Inc., Corning, NY) in supplemented basal medium with Shield1 when applicable. The cells can be cultured for 2-4 weeks. For induction of DIOLs with somatic cells, 50,000 ES cells and 30,000-75,000 E12.5 female gonadal somatic cells can be transferred into the low-cell-binding U-bottom 96-well plate in S10 medium (with 0.5 μM of Shield1 when induction is required). At 2 days of culture, aggregates are placed on TRANSWELL®-COL® membranes and cultured for 28 days (with Shield1 if needed).

For in vitro growth (IVG) culture of DIOLs, individual follicles can be manually isolated using an electronically sharpened tungsten needle. The isolated DIOLs can be cultured on TRANSWELL®-COL® membranes in IVG medium. The IVG medium can comprise a basal medium supplemented with FCS, polyvinylpyrrolidone, ascorbic acid, glutamine supplement, antibiotics, 2-mercaptoethanol, and sodium pyruvate. The IVG medium can further comprise an inducer of FSH signaling and at least one inducer of TGFβ signaling. At 2 days of culture, the at least one inducer of TGFβ signaling can be withdrawn from the medium. The follicles can be incubated in collagenase. The follicles can be washed with basal media supplemented with FCS and follicles can be cultured in IVG without inducers of TGFβ signaling for 7-11 days.

### In Vitro Embryo Production

There are several methods known in the art for generating embryos from the gamete-like cells. Standard methods of ICSI can be used to inject spermatids of the present teachings into oocytes from animals with desirable genetics or oocyte-like cells or DIOLs created in accordance with the present teachings. Alternatively, or in addition, oocyte-like cells or DIOLs of the present teachings can be used for in vitro fertilization with sperm obtained from animals with desirable genetics.

### Other Uses for Livestock Embryonic Stem Cells

Cultured meat (or lab-grown meat) is edible protein derived from stem cells and may be a “cruelty free” alternative to naturally grown meat acceptable to some vegetarians and vegans. In general, lab grown meat is produced by culturing stem cells such that they differentiate into cells edible by humans such as such as muscle cells, fat cells, bone cells, and/or cartilage cells. In some embodiments, differentiated cells are organ cells such as, for example, striated or skeletal muscle cells, smooth muscle cells, cardiac muscle cells, spleen cells, thymus cells, endothelial cells, blood cells, bladder cells, liver cells, kidney cells, pancreas cells, lung cells, or any combination thereof. Alternatively, the desired cell type is sometimes an intermediate cell type such as an adult stem cell or progenitor cell useful for generating the fully differentiated cell type. In some embodiments, growth media suitable for the growth of stem cells in vitro may be used to culture the isolated stem cells. Suitable culture media are discussed in published application US 2011/0301249 A1 by Challakere.

Once differentiated, cultures are transitioned to 3-dimensional culture. Methods of transitioning the cells to 3-dimensional culture are discussed in WO/2018/227016 by Wild Type, Inc. 3-dimensional structures for culturing are known in the art, and include, but are not limited to, edible microcarriers, hydrogels tubes and hydrogel matrices, decellularized plant materials, plant based hollow fibers, cellulose membranes, collagen blended hollow fibers, and edible hollow fibers comprising hydrocolloids and proteins. In some embodiments, cells are grown in suspension culture on micro-scaffolds that comprise at least one natural protein with texture-modifying properties. Micro-scaffolds of varying compositions can be used to produce a desired texture and/or consistency in the final food product. In various embodiments, textured vegetable protein such as soy protein is used. Micro-scaffolds optionally comprise at least one filler or binder material for providing texture to the food product. In some configurations, micro-scaffolds are made of materials that biodegrade such that the finished food product no longer has any micro-scaffold structures remaining. For example, a population of cells is seeded onto micro-scaffolds in a bioreactor. As the cells adhere to the micro-scaffolds and proliferate, the micro-scaffolds gradually biodegrade until all that remains are the clumps of cells that are now adhered to each other and the extracellular matrix materials that they have secreted. Accordingly, micro-scaffolds (and also larger 3-D scaffolds) can be used to guide the structure of the resulting cultured food product but do not remain in the food product for consumption by a human. Alternatively, micro-scaffolds and 3-D scaffolds may comprise materials that do not biodegrade and/or remain in the cultured food product for consumption. For example, certain materials described herein can be used to generate the scaffolds in order to confer a particular structure, texture, taste, or other desired property. In some embodiments, the cells can be grown on a 3-dimensional scaffold made of cellular tissue. In some embodiments, the 3-dimensional cellular tissue can comprise muscle cells including skeletal muscle cells, smooth muscle cells and satellite cells. In some embodiments, the 3-dimensional multi-type cellular tissue can comprise fat cells (e.g., adipocytes). In some embodiments, the 3-dimensional multi-type cellular tissue can comprise an extra cellular matrix secreted by specialized cells (e.g., fibroblasts). In some embodiments, the 3-dimensional multi-type cellular tissue can comprise endothelial cells or capillary endothelium formed by endothelial cells, including, but not limited to aortic endothelial cells and skeletal microvascular endothelial cells. In some embodiments, the 3-dimensional multi-type cellular tissue can further comprise an extra cellular matrix. In some embodiments, the 3-dimensional multi-type cellular tissue can further comprise adipocytes. In some embodiments, the 3-dimensional multi-type cellular tissue can further comprise capillaries. In some embodiments, progenitor cells can be produced as described herein. In some configurations, the progenitor cell can be differentiated in a monoculture and then can be incubated on a 3-dimensional porous scaffold with a plurality of cells. A nonlimiting example includes, but is not limited to, culturing and differentiating an embryonic stem cell of the present teachings into a mesenchymal stem cell, and then differentiating the mesenchymal stem cell into a myoblast cell, and then culturing differentiated myoblast on a 3-dimensional porous scaffold. Detailed culture methods and potential porous scaffolds are described in WO/2019/016795 to TECHNION RESEARCH & DEVELOPMENT FOUNDATION LIMITED.

Growth factors that can be used to differentiate cells into lab grown meat can include, such as, but without limitation platelet-derived growth factors (PDGF), insulin-like growth factor (IGF-1). PDGF and IGF1 are known to stimulate mitogenic, chemotactic and proliferate (differentiate) cellular responses. The growth factor can be one or more of the following: PDGF, e.g., PDGF AA, PDGF BB; IGF, e.g., IGF1, IGF2; fibroblast growth factors (FGF), e.g., acidic FGF, basic FGF, b-endothelial cell growth factor, FGF4, FGF5, FGF6, FGF7, FGF8, and FGF9; transforming growth factors (TGF), e.g., TGF-P1, TGF b [symbol] 0.2, TGF-p2, TGF-p3, TGF-p5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor. In some embodiments, the growth factors can be an inducer of EGF signaling, an inducer of insulin signaling, or a combination thereof. Exemplary growth factors include EGF, IGF1, or a combination thereof. Procedures for differentiating bovine cells on porous scaffolds are described in WO/2019/016795 to TECHNION RESEARCH & DEVELOPMENT FOUNDATION LIMITED.

In some embodiments, the lab meats can be organ meats. For example, to make lab grown liver, embryonic stem cells can be differentiated into hepatocytes by first differentiating into endoderm progenitor cells as described herein. The progenitor endoderm cells can then be differentiated into hepatic endoderm by treating with an inducer of bFGF signaling and an inducer of TGFβ signaling. The hepatic endoderm cells can then be differentiated into immature hepatocyte-like cells by treating with hepatocyte growth factor (HGF). These cells can then be treated with hepatocyte growth factor, Oncostatin M, and a corticosteroid such as dexamethasone. The resulting mature hepatic cell-like cells can then be grown in a suspension culture comprising an edible scaffold. The cells can then colonize the scaffold to create lab grown liver. (Adapted from a protocol on abcam.com.) Alternatively, hepatocytes may be induced from progenitor cells by causing the progenitor cells to express HNF1A, FOXA1, and HNF4A (such methods are described in WO/2018/227016 by Wild Type, Inc. Other suitable media inducers are discussed in WO/2018/227016 by Wild Type, Inc.

In various embodiments, the lab grown meats can be skeletal muscle meats. In some embodiments, skeletal muscle cells can be derived from embryonic stem cells by treating the cells with an inducer of FGF signaling and an inducer of EGF signaling. In some configurations, the inducer of FGF signaling can be FGF and the inducer of EGF signaling can be EGF. Detailed methods of induction are described in U.S. Ser. No. 11/001,803B2 to Association Francaise Contre les Myopathies. In some embodiments, livestock embryonic stem cells (such as bovine or porcine embryonic stem cells) can be induced to become mesoderm-like stem cells according to the present teachings. Alternatively, the embryonic stem cells can be induced to form mesoderm-like cells such as mesenchymal stem cell-like cells by administering a TGF-β signaling inducer. In some embodiments, the TGF-β signaling inducer can be BMP4. The cells can then be grown feeder free on plates coated in fibronectin or vitronectin. After forming trophoblasts, the cells can then be grown in serum or serum replacement containing growth medium and will differentiate into mesenchymal stem cell-like cells. Further methods of inducing embryonic stem cells into mesenchymal stem cell-like cells are discussed in U.S. Ser. No. 10/226,488B2 to the University of Connecticut. The mesenchymal-like stem cells can then be induced into skeletal muscle cells. The skeletal muscle cells can then be converted to 3-dimensional culture as discussed supra.

### EXAMPLES

The present teachings including descriptions provided in the Examples that are not intended to limit the scope of any claim or embodiment. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example illustrates the creation of a plurality of embryos for increasing NM$ in a cattle herd.

A dairy cattle production herd has a median NM$ of 700, with the top 10% of animals having a NM$ of over 850. Female bovines with a NM$ over 850 are synchronized and mature oocytes are collected via standard ovum pickup. Semen is collected from bulls in the herd with a NM$ over 850 using standard methods. Embryos from a variety of different crosses between the females and the bulls are created via in vitro fertilization.

### Example 2

This example illustrates the creation of a plurality of embryos for increasing pork production in a swine herd.

The top 10% of animals are selected from a swine production herd using a custom index prioritizing back fat, loin depth, and fertility traits. Oocytes are collected from the females via standard ovum pickup. Semen is collected from the males via standard methods. Embryos from a variety of different crosses between the females and males are created via in vitro fertilization.

### Example 3

This example illustrates the creation of embryonic stem cell lines from bovine embryos.

The inner cell mass is dissected out from each embryo produced in Example 1. Zona denuded blastocyst stage embryos are placed in PBS and dissected out using a microblade connected to an NT88-V3 high precision micromanipulator (Nikon) attached to a TE2000-U inverted microscope (Nikon). Cells are then seeded into a well from a culture plate on a single layer of MEF feeder cells and cultured in CTFR base medium supplemented with 20 ng/mL human FGF2 and 2.5 μM IWR1. Cells are incubated at 37° C.-38.5° C., 5% CO₂ for 48 hours. Cells that fail to adhere to the feeder layer are pressed against the feeder layer using a 22-gauge needle under a microscope. The media is changed daily until outgrowths appear. After 5 days, the outgrowths are treated with 10 μM Y-27632, trypsin dissociated, and passaged onto fresh feeder cells.

### Example 4

This example illustrates establishment of porcine embryonic stem cell lines.

Blastocysts from embryos created in Example 2 are used to obtain ICMs by placing them in Ca²⁺-TL-HEPES medium and then dissecting out the ICMs via microsurgery using ophthalmic scissors. Isolated ICMs are cultured for 7 days on STO cells in pEPSCM comprising per 500 ml N2B27 basal media: 0.2 μM CHIR99021 (a GSK3 inhibitor; Tocris, cat. no. 4423), 0.3 μM WH-4-023 (an SRC inhibitor; Tocris, cat. no. 5413), 2.5 μM XAV939 (a tankyrase inhibitor; Sigma cat. no. X3004) or 2.0 μM IWR-1 (a WNT inhibitor; Tocris, cat. no. 3532), 65.0 pg/ml vitamin C (Sigma, cat. no. 49752-100G), 10.0 ng/ml LIF (Stem Cell Institute (SCI), University of Cambridge), 20.0 ng/ml Activin (SCI) and 0.3% FBS (Gibco, cat. no. 10270)+10 μM Y27632 (ROCK inhibitor) (Gao, X. et al., Nature Cell Biology, 2019, 21, 687-699). Outgrowths appear after 2 days. The outgrowths are mechanically isolated and reseeded onto fresh STO cells in pEPSCM. The cells form well-defined colonies 3 days later.

### Example 5

This example illustrates the selection of bovine embryonic stem cells for in vitro breeding of the next generation.

An aliquot of each stem cell line is taken and DNA extracted using standard methods. The DNA is then genotyped using the BovineSNP50 v3 BeadChip microarray kit according to the manufacturer's directions. These SNP genotypes are then analyzed to calculate the predicted NM$ for each cell line. The top 10% NM$ cell lines are selected for gametogenesis for creation of the next generation. Crosses between the generated gametes or between the gametes and other animals are then planned.

### Example 6

This example illustrates the selection of porcine embryonic stem cells for in vitro breeding of the next generation.

An aliquot of each stem cell line is taken and DNA extracted using standard methods. The DNA is then genotyped using the PorcineSNP60v2 BeadChip according to manufacturer directions. The resulting SNPs are then analyzed using a custom index prioritizing back fat, loin depth, and fertility traits. The top 10% of lines according to this index are then selected for gametogenesis for creation of the next generation of cell lines.

### Example 7

This example illustrates a method of inducing differentiation of bovine embryonic stem cells into PGCLCs.

Embryonic stem cells from the cell lines generated in Example 3 are dissociated with TrypLE™ (Thermo Fisher Scientific, Waltham, MA; Invitrogen 12604-021) and seeded onto dishes precoated with collagen IV to create feeder free cultures. These cultured ESCs are then induced into epiblast-like cells (EpiLCs) in N2B27 culture medium comprising 20 ng/ml activin A, 12 ng/ml bFGF, and 1% KNOCKOUT® serum replacement (KSR). These cells are incubated for 3 days. This medium is changed daily.

This floating culture of EpiLCs is then plated in a well of a low-cell-binding U-bottom 96-well plate (NUNC) in GK15 media (GMEM [Invitrogen] with 15% KSR, 0.1 mM NEAA, 1 mM sodium pyruvate, 0.1 mM 2-mercaptoethanol, 100 U/ml penicillin, 0.1 mg/ml streptomycin, and 2 mM L-glutamine). The GK15 media is further supplemented with 500 ng/ml BMP4 (R&D Systems), 1000 U/ml LIF (Invitrogen), 100 ng/ml SCF (R&D Systems), 500 ng/ml BMP8b (R&D Systems), and 50 ng/ml EGF (R&D Systems). The cells are maintained at 37° C.-38.5° C. at 5% CO₂. Differentiated PGCLCs are separated from undifferentiated EpiLCs via FACs selection using a-SSEA-1.

### Example 8

This example illustrates an alternate method of inducing differentiation of bovine embryonic stem cells into PGCLCs.

Differentiation of embryonic stem cells from the lines generated in Example 3 into EpiLCs is carried out as described in Example 7.

These induced EpiLCs are then transiently transfected with vectors that cause the expression of PRDM1, PRDM14 and TFAP2C. These cells are incubated for 2 days. Differentiated PGCLCs are separated from undifferentiated EpiLCs via FACs selection using c-kit surface markers.

### Example 9

This example illustrates the differentiation of porcine embryonic stem cells into PGCLCs.

Embryonic stem cells from the cell lines generated in Example 4 are adapted to feeder free culture on a dish coated in gelatin. Cells are then disassociated with trypsin and treated with N2B27 media comprising 50 ng/ml BMP4 (PeproTech), 50 ng/ml, BMP8b (R&D), 50 ng/ml SCF (PeproTech), 50 ng/ml EGF (PeproTech), and 1000 U/ml LIF (Gibco). These cells are incubated for 2 days. Differentiated PGCLCs are separated from undifferentiated EpiLCs via FACs selection using TNAP surface markers.

### Example 10

This example illustrates an alternate method of differentiating ESC into PGCLCs.

Porcine ESCs from Example 4 are transiently transfected with vectors that induce the transcription of NANOG, BLIMP1, TFAP2C, and SOX17. The cells are cultured in a medium comprising Advanced RPMI 1640, 1% B27 supplement, 1.0× glutamine penicillin-streptomycin, 1.0×NEAA, 0.1 mM, 2-mercaptoethanol and the following cytokines: 500.0 ng/ml BMP2, 10.0 ng/ml LIF, 100.0 ng/ml SCF, 50.0 ng/ml EGF and 10.0 μM Y27632. Undifferentiated cells are separated from PGCLCs via FACs selection using EMA-1 cell surface markers.

### Example 11

This example illustrates a method of differentiation of bovine male PGCLCs into SSCLCs.

Male PGCLCs from Example 7 are dissociated and plated in gelatin-coated wells of 12-well plates (2×10⁵ cells/well) with DMEM medium containing 15% FBS, NEAA, L-glutamine, penicillin/streptomycin, β-mercaptoethanol, 2 μM RA, 4 ng/ml GDNF, and 1 μM testosterone (called RGT medium).

Meiosis specific markers RecA and Stra8 and spermatogonia stem cells (SSC) specific markers are monitored and compared to day 0 PGCLC in culture to confirm PGCLC differentiation to spermatogonia stem cell-like cells (SSCLC). (See Zhou, Q., et al., Cell Stem Cell, 2016, 18, 330-340).

### Example 12

This example illustrates a method of differentiation of porcine male PGCLCs into SSCLCs.

Porcine embryonic testis somatic cells are prepared by isolating porcine embryonic testis from a NANOS2 knockout embryo. The cells are then dispersed with 1 mg/ml collagenase type IV at 37° C.-38.5° C. for 10 min, followed by dissociation in 0.25% trypsin/1 mM EDTA for 10 min at 37° C.-38.5° C. A single cell suspension is obtained by filtering through a cell strainer, and cells are collected by centrifugation.

These cells are then mixed in a 1:1 ratio with male PGCLCs from Example 9. This mixture is then cultured in α-MEM supplemented with 10% KSR, 20 ng/ml each of BMP-2, BMP-4, and BMP-7 (R&D Systems), 10 μM retinoic acid (Sigma), and 100 ng/ml activin A (R&D Systems).

Meiosis specific markers RecA and Stra8 and spermatogonia stem cells (SSC) specific markers are monitored and compared to day 0 PGCLC in culture to confirm PGCLC differentiation to spermatogonia stem cell-like cells (SSCLC). (See Zhou, Q., et al., Cell Stem Cell, 2016, 18, 330-340).

### Example 13

This example illustrates an alternate method of differentiating PGCLCs into SSCLCs.

Porcine PGCLC are reseeded on a feeder layer (3×10⁵ cells per well of a 6-well-plate) in NEUROBASAL™ (Gibco, Waltham, Massachusetts, USA) and DMF/12 medium (Hyclone, Logan, Utah, USA) (1:1), supplemented with 100×N-2 Supplement (Gibco) and 50×B-27™ Supplement (Gibco). This NB27 media is further supplemented with 25 ng/ml BMP4 (PeproTech), 20 ng/ml bFGF (PeproTech), 40 ng/ml GDNF (PeproTech), 10 ng/ml LIF (Miltenyi Biotec, Bergisch Gladbach, Germany), 12 μg/ml insulin (Sigma-Aldrich), and 15% KSR.

Meiosis specific markers RecA and Stra8 and spermatogonia stem cells (SSC) specific markers are monitored and compared to day 0 PGCLC in culture to confirm PGCLC differentiation to spermatogonia stem cell-like cells (SSCLC). (See Zhou, Q., et al., Cell Stem Cell, 2016, 18, 330-340).

### Example 14

This example illustrates a method of differentiating bovine SSCLCs into spermatid like-cells.

SSCLCs from Example 10 are cultured in αMEM containing 10% KSR, 10 mM testosterone (Acros Organics), 200 ng/ml FSH (Sigma), and 50 mg/ml BPE (CORNING® Life Sciences). The medium is changed every 2 days. Cells are cultured in 5% CO₂ at 37° C.-38.5° C.

### Example 15

This example illustrates a method of differentiation of porcine SSCLCs into spermatid-like cells.

SSCLC from Example 11 are reseeded on a feeder layer in a 6-well-plate in N2B27 basal culture medium supplemented with 2 nM RA (Sigma-Aldrich) and 15% KSR. Cells are cultured in 5% CO₂ at 37° C.-38.5° C.

### Example 16

This example illustrates the formation of oocytes from PGCLCs in reconstituted ovaries.

Female PGCLCs from Example 8 (bovine) are reconstituted with cattle female gonadal somatic cells in 1:1 ratio in a low-binding U-bottom 96-well plate (NUNC) in GK15 culture media supplemented with 1 μM retinoic acid. The bovine female gonadal somatic cells are prepared by isolating embryonic ovaries, dispersing them with 1 mg/ml collagenase type IV at 37° C.-38.5° C. for 10 min, followed by digestion in 0.25% trypsin/1 mM EDTA for 10 min at 37° C.-38.5° C. A single cell suspension is obtained after filtration through a cell strainer, and cells are collected by centrifugation. These cells are then depleted of endogenous primordial germ cells (PGC) by magnetic cell sorting (MACS) using a PGC specific protein marker antibody conjugated with magnetic beads. This PGC depletion step can be obviated by using ovaries isolated from embryos with a female germ cell specific knockout (for example NANOS3 knockout animals). These depleted bovine somatic cells are then mixed with the female PGCLCs from Example 8 to form reconstituted ovaries.

The reconstituted ovaries are placed on TRANSWELL®-COL membranes (CORNING® Incorporated, Corning, NY) soaked in α MEM-based in vitro differentiation (IVDi) medium. The IVDi medium comprises: α MEM supplemented with 2% FCS, 150 μM ascorbic acid (Sigma), 1× GLUTAMAX™, 1× penicillin/streptomycin and 55 μM 2-mercaptoethanol (Life Technologies). At 4 days of culture, the culture medium is changed to StemPro-34-based IVDi medium comprising StemPro-34 SFM (Life Technologies) supplemented with 10% FCS, 150 μM ascorbic acid, 1× Glutamax, 1× penicillin/streptomycin, and 55 μM 2-mercaptoethanol. From day 7 to day 10 of culture 500 nM ICI182780 is added to the StemPro-34-based IVDi medium with each media change. At about 21 days of culture, individual secondary follicle like structures (2FLs) form.

Electrically sharpened tungsten needles can be used for the isolation of individual secondary follicle like structure (2FLs). Interstitial cells between 2FLs can be removed. The 2FLs can be separated from the Reconstituted ovaries (rOvaries), dissociated, and placed at largely regular intervals on TRANSWELL®-COL® membranes.

The single 2FLs on the TRANSWELL®-COL® membranes are soaked in IVG-α MEM medium comprising a MEM supplemented with 5% FCS, 2% polyvinylpyrrolidone (Sigma), 150 μM ascorbic acid, 1× GLUTAMAX™, 1× penicillin/streptomycin, 100 μM 2-mercaptoethanol, 55 μg/ml sodium pyruvate (Nacalai Tesque),0.1 IU/ml follicle-stimulating hormone (Follistim; MSD), 15 ng/ml BMP15 and 15 ng/ml GDF9 (R&D Systems). At 2 days of culture, BMP15 and GDF9 are withdrawn from the medium and then follicles are incubated in 0.1% Type IV Collagenase (MP Biomedicals). After washing with α MEM supplemented with 5% FCS several times, the follicles are cultured in IVG-α MEM without BMP15 and GDF9. At 11 days of culture, cumulus-oocyte complexes (COCs) grown on the membrane are picked up by a fine glass capillary. Cumulus-oocyte complexes are transferred to IVM medium: a MEM supplemented with 5% FCS, 25 μg/ml sodium pyruvate, 1× penicillin/streptomycin, 0.1 IU/ml FSH, 4 ng/ml EGF, and 1.2 IU/ml-1 hCG (gonadotropin; ASKA). At 16 h of culture, swollen cumulus cells are stripped from the oocytes by treating with hyaluronidase (Sigma), and then MII oocytes are determined by 1st polar body extrusion under a microscope.

### Example 17

This example illustrates a method of inducing oocytes directly from embryonic stem cells.

ESCs from Example 4 are passaged under feeder-free conditions at least three times.

For the long-term induction of directly induced oocyte-like cells (DIOLs) without somatic cells, 50,000 ES cells are transferred into the low-cell-binding U-bottom 96-well plate in S10 medium (StemPro-34 SFM (Life Technologies). The S10 medium is supplemented with 10% FCS, 150 μM ascorbic acid, 1× GLUTAMAX™, 1× penicillin/streptomycin and 55 μM 2-mercaptoethanol, 150 ng/ml SCF, and 10 nM Y-27632 (Wako). At 2 days of culture, aggregates are placed on TRANSWELL®-COL® membranes (CORNING®) in S10 medium. Non-integrative plasmids comprising FIGLA, SOHLH1, LHX8, NOBOX, STAT3, TBPL2, DYNLL1, and SUBJ coding cassettes on the CMV promoter are transiently transfected into the culture cells at intervals allowing the cells to express them for three weeks.

For in vitro growth (IVG) culture of DIOLs, individual follicles are manually isolated using a sharpened tungsten needle. The isolated DIOLs are cultured on TRANSWELL®-COL® membranes in IVG medium. The IVG medium comprises αMEM supplemented with 5% FCS, 2% polyvinylpyrrolidone (Sigma), 150 μM ascorbic acid, 1× GLUTAMAX™, 1× penicillin/streptomycin, 100 μM 2-mercaptoethanol, 55 μg/ml sodium pyruvate (Nacalai Tesque), 0.1 IU/ml follicle-stimulating hormone (Follistim; MSD), 15 ng/ml BMP15 and 15 ng ml/GDF9 (R&D Systems). At 2 days of culture, BMP15 and GDF9 are withdrawn from the medium and then follicles are incubated in 0.1% type IV collagenase. After washing with αMEM supplemented with 5% FCS several times, the follicles are cultured in IVG-αMEM without BMP15 and GDF9 for 7-11 days until DIOLs are formed.

### Example 18

This example illustrates in vitro fertilization using bovine gametes generated in vitro.

In order to create a plurality of embryos for the next round of in vitro breeding, a plurality of spermatid-like cells from Example 12 is injected into a plurality of oocytes from Example 14 using standard methods of ICSI (one spermatid-like cell per oocyte). Each of the resulting embryos that reach the blastocyst stage is then used to create a cell line as described in Example 3 and another cycle of in vitro breeding is begun.

### Example 19

This example illustrates in vitro fertilization using porcine gametes that were created in vitro.

In order to create a plurality of embryos for the next round of in vitro breeding, a plurality of spermatid-like cells from Example 11 is injected into a plurality of oocytes from Example 15 using standard methods of ICSI (one spermatid-like cell per oocyte). Each of the resulting embryos that reach the blastocyst stage is then used to create a cell line as described in Example 4 and another cycle of in vitro breeding is begun.

### Example 20

This example illustrates nuclear transfer of selected bovine embryonic stem cell lines to create an animal of the same genotype.

For the top 1% of NM$ bovine embryonic stem cell lines created in Example 5 or Example 16, nuclear transfer is performed in a similar method to that published in Ross and Cibelli, Methods Mol Biol. 636. 155-77 (2010) and Ross et al., BioTechniques 41:741-750 (2006). See also U.S. Pat. No. 6,011,197 issued Jan. 4, 2000, to Strelchenko et al.

Briefly, donor cells are dissociated by treatment with 10 IU/mL pronase in HH media for 5 min. Oocytes are harvested from either slaughterhouse-derived ovaries or from live animals by ultrasound-guided oocyte aspiration. The oocytes are matured in vitro and enucleated. A single cell from a selected embryonic stem cell culture is inserted into the perivitelline space of the enucleated oocyte using a 20-μm (internal diameter) glass pipet. Oocyte-cell fusion is induced using a square DC pulse generator. Fused oocytes are activated using ionomycin and cultured under standard conditions. At 48 h after activation, noncleaved embryos are removed from culture and at 72 h after activation, the culture medium is optionally supplemented with serum and cultured for seven days before being recovered and implanted in synchronized recipients. Calves are born normally to the surrogate mother and are genetically identical to the embryonic stem cells. The high availability of bovine oocytes and the relatively higher efficiency levels usually obtained in cattle provide for the use of SCNT for both commercial and research purposes.

### Example 21

This example illustrates nuclear transfer of porcine embryonic stem cells.

Porcine embryonic stem cells are grown to confluence and forced to arrest in G1 or G2 of the cell cycle. Oocytes are obtained and prepared and the nucleus transferred as described in Example 20.

### Example 22

This example illustrates exemplary media for deriving livestock embryonic stem cells.

N2B27 base medium (NBM) was created by mixing 40 mL of DMEM/F12 Medium (Gibco 11320-033), 10 mL of 100 mg/ml BSA stock resuspended in DMEM/F12 Medium, 500 μL N-2 Supplement (100×; Gibco 17502-048), 50 mL Neurobasal Medium (Gibco 21103-049), 1 mL B-27 Supplement (50×; Gibco 17504-044), 1 mL MEM Non-essential Amino Acid Solution, 1 mL GLUTAMAX™ Supplement (100× catalog: Gibco 35050-061), 100 U/ml mL Penicillin-Streptomycin (10,000 U/mL; Gibco 15140-122), 10 μL 2-Mercaptoethanol 1M Stock (final concentration 0.1 mM; Sigma M6250). Once mixed, the medium was filter sterilized.

For NBFR medium, this base medium was supplemented with 20 ng/mL recombinant human fibroblast growth factor (FGF; Peprotech 100-18B) and 2.5 μM IWR1 (SIGMA I0161).

### Example 23

This example illustrates a method of deriving bovine embryonic stem cells.

A layer of MEF feeder cells was plated at 118,000 cells per well in a 4 well plate and cultured on DMEM media supplemented with 10% fetal bovine serum (FBS), 1% Pen/Strep (Gibco 15140-122), and 1% GLUTAMAX™. Once ready for seeding, the feeder cells were washed in phosphate buffered saline (PBS) then covered in a half volume of media. The media was removed before addition of the blastocysts.

Whole blastocysts (with the zone pellucida removed) were added to the feeder plates with medium supplemented with 10 μM Y-27632 ROCK inhibitor (ENZO, ALx-270-333-M005; ROCKi). 24 hours after plating, embryos that had not attached to the MEFs were pressed down using a 32 g needle until they are stuck to the plate.

Medium was changed 48 hours after plating and daily afterwards. Six days after plating, the cells were passaged (see Example 24) to a new well (even if no overt cell growth was evident) without splitting (at a ratio of 1:1 on a same size well). After six days the cells were passaged again (even if no overt cell growth was evident) from a 4 well plate to 12 well plate. The cells were then passaged 3-5 days after second passage, depending on growth, from a 12 well plate to 6 well plate. After this passage, the cells were typically passaged every 3-4 days at 1:5 to 1:10 ratio depending on growth. Cell lines that did not form colonies after 4-5 passages were discarded. The present inventors have established 76 bovine embryonic stem cell lines from various sources as shown in Table 1.

TABLE 1
ESC Lines Established from Multiple Sources

| Genetics | # of ESC Lines Established |
|---|---|
| Abattoir | 41 |
| Elite ABS Genetics, Dairy (DeNovo) | 18 |
| Elite ABS Genetics, Beef (NuEra) | 17 |

Starting materials were either oocytes or embryos that were obtained from abattoir ovaries (in vitro), through ovum pickup technique (in vitro), or flushed embryos (in vivo).

### Example 24

This example illustrates maintenance of cell lines established in Example 23.

Cells were maintained by passage every 3-5 days depending on growth. The growth medium was removed. Next, the wells of the plate were washed with PBS and detached from the plate using 500 uL per well of 6-well plate of TrypLE and incubated 2 to 3 min in the 37° C. incubator. 5 ml of NBFR media (see Example 22) were added and the mixture was transferred to a 15 ml falcon tube. The tube was centrifuged at room temperature at 400×g for 4 minutes. New feeder cell wells were prepared by washing with PBS as described above during this spin. After the spin, the bulk supernatant was discarded from the falcon without disturbing the pellet. The pellet was then resuspended in pre-warmed NBFR media and the appropriate number of cells was added to the pre-washed well (depending on the needed split). A final concentration of 10 μM of ROCKi was optionally added to increase cell survival rate when needed. Fresh media (without ROCKi) was added after 24 hours. Media was then changed on the cells daily.

For cryopreservation of the embryonic stem cells, the cells were washed as described above for passaging, except that after the spin and removal of the supernatant, the cells were then resuspended in 900 μl of media containing 1 μl ROCKi and 100 μl DMSO. The cells were then slow frozen and then transferred to −80° C. for approximately 24 hours before storage in liquid nitrogen.

For thawing of cryopreserved cells, frozen cells were heated in a 37° C. water bath until only a small ice crystal remained; the thawed cells and freezing solution were transferred to a conical tube and then 5 mL of pre-warmed NBFR media was added dropwise. The cells were then centrifuged at room temperature at 400×g for 4 minutes. Fresh feeder cell wells were washed during the spin. After the spin, the supernatant was discarded. The pellet was resuspended in 2 ml of media containing 2 ul ROCK inhibitor and then transferred to the pre-washed wells. After 24 hours, the media with ROCKi was removed and replaced with fresh media without ROCKi. Media changes and passaging were then performed as above.

### Example 25

This example illustrates medium and procedures used with feeder free embryonic stem cell culture.

For ES Medium, the N2B27 base medium of was supplemented with 20 ng/ml bFGF (Peprotech, 100-18B), 2.5 μM IWR1 (Selleck Chemicals, 1127442-82-3), 20 ng/ml activin A (R&D, Cat. 338-AC-010).

For ES Passage medium, ES medium was further supplemented with 10 μM Y-27632 ROCK inhibitor (ENZO, ALx-270-333-M005; ROCKi).

For feeder free cultures, non-coated culture dishes were coated with extracellular matrix proteins. VITRONECTIN XF™ (Trademark owned by Nucleus Biotech, Inc, San Diego, CA; purchased from Stemcell Technology, Cat. 07180) was used to coat a 6 well plate according to the manufacturer's directions and then incubated at room temperature for one hour. The plate was then washed in DPBS (Gibco) and then 2 ml of ES medium was added to each well.

### Example 26

This example illustrates conversion of embryonic stem cell cultures from feeder cell cultures to feeder free cultures.

ESCs on feeder cell culture as established in Example 23 were washed with 1 mL/well of D-PBS (without Ca++ and Mg++). 1 mL/well of ReLeSR™ (STEMCELL Technologies Canada Inc, Vancouver, Canada) was then added to each well and the released embryonic stem cells were removed with the medium within 1 minute and transferred to a fresh container. The cells were then incubated at room temperature or 37° C. for 5-7 minutes. 1 mL/well of ES culture medium was then added to each well. The colonies were then detached by tapping the side of the plate according to manufacturer directions for approximately 30-60 seconds. Cell aggregate sizes were checked to make sure they were of a size recommended by the ReLeSR™ manufacturer (approximately 50 to 200 μm) prior to plating. The detached cell aggregates were then transferred a 15 mL tube. The cell mixture was pipetted up and down 2-5 times to ensure the breakup of any large aggregates. The cell aggregate mixture was then plated at the desired density (1:2-1:4 ratio/well, depending on the application) onto pre-coated wells containing ES Passage medium. The volume was brought up to the appropriate volume with passage medium as needed. The plates were then placed in a 37° C. incubator, moved in several quick, short, back-and-forth, and side-to-side motions to evenly distribute the cell aggregates, and then left in place for 24 hours. After 24 hours, the medium was changed daily with ES culture medium and the cultures were visually assessed to monitor growth.

### Example 27

This example illustrates maintenance of feeder free bovine embryonic stem cell cultures.

Like all culture cells, embryonic stem cells require passaging when the cells approach confluence. To passage, the cell culture media was removed and the cells were washed with 1 mL/well of DPBS. After washing the cells were treated with 1 mL/well of ReLeSR™ for up to 1 minute and most of the ReLeSR™ was removed, except for a small amount. The cells were then incubated at room temperature for 5-7 minutes. Then, 1 ml/well of ES passage medium (see Example 25) was added to each well. The colonies were then detached by tapping the side of the plate according to manufacturer directions for approximately 30-60 seconds. Cell aggregate sizes were checked to make sure they were of a size recommended by the ReLeSR™ manufacturer prior to plating. The detached cell aggregates were then transferred a 15 mL tube.

The cell mixture was pipetted up and down 2-5 times to ensure the breakup of any large aggregates. The cell aggregate mixture was then plated at the desired density (1:2-1:4 ratio/well, depending on the application) onto pre-coated wells containing ES passage medium (see Example 25). The volume was brought up to the appropriate volume with passage medium as needed. The plate was then transferred to a humidified incubator (38.5° C., 5% CO₂, 5% O2) and moved in several quick, short, back-and-forth, and side-to-side motions to evenly distribute the cell aggregates, and then the cells were allowed to settle for 24 hours. The cells were then maintained with daily cell free culture medium changes and visually assessed to monitor growth.

For cryopreservation of feeder free maintained bovine embryonic stem cells, the cell culture media was removed and the cells were washed with 1 mL/well of DPBS. The DPBS was then removed; 1 mL of ReLeSR™ was added to each well aspirated within 1 minute and most of the ReLeSR™ was removed, except for a small amount. The cells were then incubated at room temperature for 5-7 minutes. The plate was then incubated at room temperature for 5-7 minutes, per manufacturer instructions. 1 ml of passage medium was added to each well (see Example 25). The colonies were then detached by tapping the side of the plate according to manufacturer directions for approximately 30-60 seconds. Cell aggregate sizes were checked to make sure they were of a size recommended by the ReLeSR™ manufacturer prior to plating. The detached cell aggregates were then transferred a 15 mL tube.

Cells were frozen in a 1 ml freezing vial in a cryopreservation mix comprising 300 μL of cell suspension, 600 μL ES passage medium, and 100 μL DMSO. Mixtures were pipetted 2-5 times to mix and break up into small aggregates. The vials were then placed in a cryobox and incubated at −80° C. overnight. The vials were then transferred to liquid nitrogen for long term storage.

### Example 28

This example illustrates morphology of bovine embryonic stem cells.

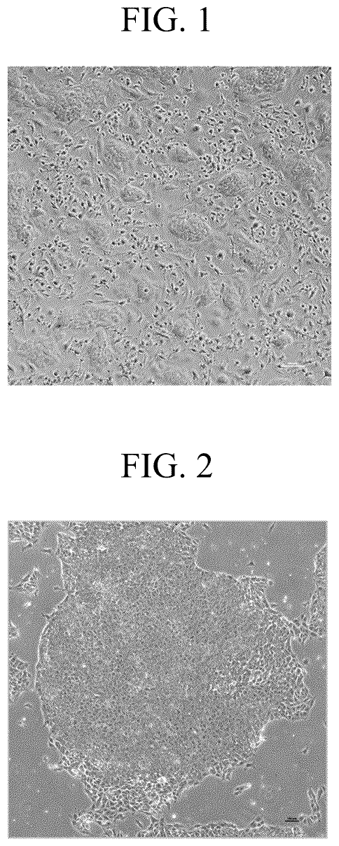

Cells grown on feeder cells as in Example 24 (FIG. 1) and under feeder free conditions as in Example 27 (FIG. 2) were observed under a Nikon ECLIPSE Ti2 inverted microscope at 40×. (Photographs were taken with NIS elements Br software.) The embryonic stem cells gathered in aggregate spots when grown with feeder cells (FIG. 1). When grown under feeder free conditions, the cells form a confluent structure (FIG. 2). (Scale bar is 100 μm.) This morphology is expected for embryonic stem cells.

### Example 29

This example illustrates that bovine embryonic stem cells have gene expression profiles consistent with pluripotent cells.

To analyze the protein expression, cell pellets of embryonic stem cells as described in Example 23 and Example 26 were thawed on ice in RIPA buffer(Sigma) containing 1× protease inhibitor cocktail (Roche Applied Science) for 30 min at 4° C. Lysates were subjected to high intensity sonication (3 sets of 30 second pulses) and the insoluble materials were removed by centrifugation at 15,000 rpm for 15 min at 4° C. The total protein concentration for supernatants was determined using a DC protein assay kit (Bio-Rad). Total 300 ng of protein extract per sample was analyzed using Capillary Western blots (Rajput, S., et al., F S Sci., 2021, 2, 33-42) for CDX2 (Abcam ab76541), GATA6 (Cell Signaling 5851T), OCT4 (Abcam ab181557), NANOG (PeproTech 500-P236), and X2 (Cell Signaling 3579T) markers. Increased expression was observed for Oct4, Sox2, and NANOG. CDX2 (specifies trophectoderm) showed minimal expression, and GATA6 (specifies primitive endoderm) expression was undetectable in the Western blot. This expression profile is expected for pluripotent embryonic stem cells.

This expression was further verified by real time PCR (RT-PCR). Primer pairs are shown in Table 2.

TABLE 2
Primers Used for RT-qPCR Analysis of Pluripotent Marker Expression

| Genbank | Target | | | Product |
|---|---|---|---|---|
| Accession | Name | SEQ ID NO | Sequence | Size |
| AM293662 | CDX2 | 1 | CGTCTGGAGCTGGAGAAGGA | 70 |
| | | 2 | CGGCCAGTTCGGCTTTC | |
| NM_214328 | GATA6 | 3 | GAGCAGCCGGAGGAGATGTACCAGAC | 110 |
| | | 4 | GGCTCAGGCCAGGGCCAGGGCGCACC | |
| NM_001025344 | NANOG | 5 | AAAGTTACGTGTCCTTGCAAACG | 73 |
| | | 6 | GAGGAGGGAAGAGGAGAGACAGT | |
| NM_174580 | OCT_4 | 7 | GTTTTGAGGCTTTGCAGCTC | 99 |
| | | 8 | CCTGCAGATTCTCGTTGTTG | |
| NM_001105463 | SOX2 | 9 | AGCGCATGGACAGCTACGCG | 127 |
| | | 10 | ATGGGCTGCATCTGAGCGGC | |
| NM_001034034.2 | GAPDH | 11 | TCATCATCTCTGGACCTTCTG | 154 |
| | | 12 | ATGCCAAAGTGGTCATGGA | |

RT-qPCR was performed to analyze the mRNA expression of all the markers. RNA was isolated using the PureLink™ RNA minikit (Thermo Fisher Scientific, Waltham, MA) from bovine fibroblasts and bovine embryonic stem cells. RNA was converted into cDNA using iScript™ cDNA synthesis kit (Bio-Rad Laboratories, Inc., Hercules, CA; 1708891) and used for qPCR using POWERTRACK™ SYBR Green Master Mix (Thermo Fisher Scientific, Waltham, MA A46012). All kits were used according to the manufacturer's directions.

Within an extract, mRNA expression for each marker was normalized to GADPH. Expression in bovine embryonic stem cells was then compared to expression in fibroblasts and presented as fold change. Results from an average of 5 ESC lines are listed in Table 3.

TABLE 3
RT-PCR Quantitation of Pluipotency Markers

| | |
|---|---|
| Bovine Fibroblast | Bovine ESC |

| | mean | SD | mean | SD |
|---|---|---|---|---|
| OCT4 | 1 | 0 | 499.6 | 182.3 |
| SOX2 | 1 | 0 | 849.9 | 295.8 |
| NANOG | 1 | 0 | 37451.2 | 13114.5 |
| CDX2 | 1 | 0 | 1.8 | 0.4 |
| GATA6 | 1 | 0 | 0.3 | 0.5 |

The transcription of pluripotency markers OCT4, SOX2, and NANOG is markedly increased in Bovine ESCs. In contrast, specification markers CDX2 and GATA6 are close in transcript abundance relative to fibroblast cells. Bovine embryonic stem cells created as described herein express pluripotency markers in a manner consistent with pluripotent cells.

### Example 30

This example illustrates spontaneous differentiation of bovine embryonic stem cells.

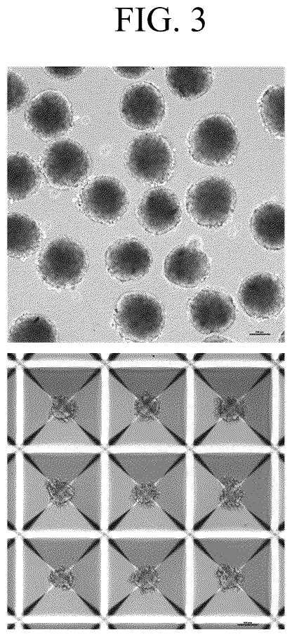

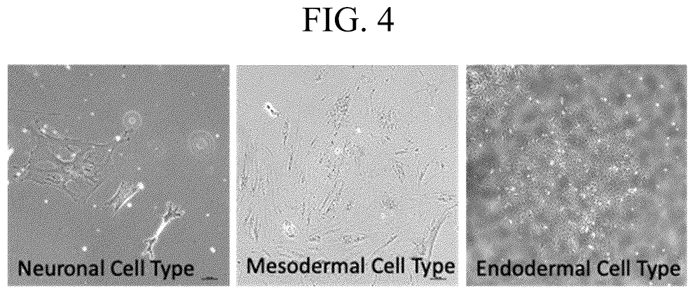

Bovine embryonic stem cells were detached from culture dishes using TrypLE™ Select (Thermo Fisher Scientific, Waldham, MA, 12563011), resuspended in CF-1 feeder cells culture medium (DMEM supplemented with 10% FBS), and then seeded into AggreWell™ 400 microwell culture dishes (STEMCELL Technologies Canada Inc., Vancouver Canada). After four days in suspension culture, embryoid bodies were formed. FIG. 3 illustrates a representative picture of the embryoid bodies at 40×. The embryoid bodies were transferred to gelatin-coated dishes and cultured for another 2 weeks prior to observation and RT-qPCR The embryonic stem cells differentiated into neuronal cells (ectoderm), mesodermal cells, and endodermal cells; representative spontaneously differentiated cells are shown in FIG. 4. The primer pairs used for RT-qPCR are listed in Table 4.

TABLE 4
RT-PCR quantitation of differentiation markers

| Genbank | Target | SEQ | | Product |
|---|---|---|---|---|
| Accession | Name | ID NO | Sequence | Size |
| NM_001034262 | AFP | 13 | CTTACACAAAGAAGGC | |
| | | | TCCTC | |
| | | 14 | CTATAATAAGGTCAGC | 143 |
| | | | CACTCCC | |
| NM_001077127.1 | TUBB3 | 15 | ACAACTTCATCTTTGGT | |
| | (TUJ1) | | CAGAG | |
| | | 16 | ATCCAAGACTGAGTCC | 91 |
| | | | ACCA | |
| NM_001034502.1 | ACTA2 | 17 | GCACCACTCTTTCTAC | |
| | (SMA) | | AATGAG | |
| | | 18 | AGTCTCAAACATAATC | 120 |
| | | | TGGGTC | |

RNA extraction and RT-qPCR was performed as described in Example 29. Within an extract, mRNA expression for each marker was normalized to GADPH. Expression in the differentiated cells was then compared to expression in undifferentiated bovine embryonic stem cells and reported as fold change. AFP was used to look for endodermal differentiation, ACTA2 to look at mesodermal differentiation, and TUBB3 to look at ectodermal differentiation. As shown in Table 5, transcript abundance of each of these markers increased relative to their abundance in bESCs.

TABLE 5
Expression of Differentiation Markers

| | |
|---|---|
| | Spontaneously |
| bESC | Differentiated Cells |

| | mean | SD | mean | SD |
|---|---|---|---|---|
| TUBB3 | 1 | 0 | 2.3 | 0.3 |
| ACTA2 | 1 | 0 | 2.6 | 0.2 |
| AFP | 1 | 0 | 1.8 | 0.4 |

These data illustrate that the embryonic stem cells of the present teachings can differentiate into all three germ layers.

### Example 31

This example illustrates the karyotypic normality of bovine embryonic stem cells.

Two cell lines, one having a male genotype and one having a female genotype, were selected for karyotyping. Briefly, cells from lines that had undergone 10 passages were prepared and treated with Colcemid, washed, and harvested. Harvested cells were treated with a hypotonic solution, fixed, stained, and analyzed. Chromosomes were mounted and then G banding was analyzed. Number, morphology, and G banding of the chromosomes were all normal. This example shows that bovine embryonic stem cells prepared according to the present teachings are closer in phenotype to animals than previous attempts at livestock embryonic stem cells.

### Example 32

This example illustrates genotyping of embryonic stem cells with elite genetics.

DNA was extracted from embryonic stem cells of the present teachings. The DNA was then submitted to Council on Dairy Cattle Breeding (CDCB) as embryonic DNA for breed registration and genotyping using a NEOGEN® GGP Bovine 100 k chip. CDCB provided Predicted Transmitting Abilities (PTAs) calculated from the SNP results. These results are reported in Table 6 and Table 7.

TABLE 6
Predicted Phenotypic Traits from Genotype (male cell lines)

| | id17 | | | |
|---|---|---|---|---|
| | HO840003236266751 | HO840003236266752 | HO840003236266753 | HO840003236266754 |
| | sex | | | |
| | M | M | M | M |
| sire17 | HO840003150687531 | HO840003150687531 | HO840003150687531 | HO840003150687531 |
| dam17 | HO840003204166547 | HO840003220285682 | HO840003214630863 | HO840003214630863 |
| NM | 939 | 1099 | 1060 | 1153 |
| PL | 6.3 | 6 | 6.1 | 5.9 |
| SCS | 2.77 | 2.8 | 2.67 | 2.83 |
| DPR | −0.3 | −0.1 | −0.2 | 0.2 |
| NM_REL | 72 | 71 | 71 | 71 |
| Milk | 1250 | 1181 | 1527 | 1483 |
| Fat | 85 | 103 | 94 | 111 |
| Pro | 46 | 48 | 55 | 59 |
| FatPct | 0.13 | 0.2 | 0.12 | 0.18 |
| ProPct | 0.02 | 0.04 | 0.03 | 0.04 |
| HCR | 0.5 | 1.3 | 0.1 | 1.5 |
| CCR | 0.3 | 1.6 | 0.5 | 1.1 |
| LIV | 3.4 | 4.1 | 3.7 | 2.8 |
| Milk_REL | 79 | 78 | 78 | 78 |
| STA | −0.95 | −2 | −1.15 | −1.26 |
| STR | −0.33 | −1.59 | −1.51 | −1.89 |
| BDE | −0.41 | −1.21 | −1.01 | −1.39 |
| DFM | 0.02 | 0.92 | 1.1 | 1.29 |
| RPA | −0.97 | −0.13 | −0.56 | 0.19 |
| TRW | −0.45 | −1.39 | −0.8 | −1.15 |
| RLS | −1.77 | 0.35 | −0.25 | −0.51 |
| RLR | 0.94 | −0.83 | −0.18 | −0.26 |
| FTA | 0.7 | −1.05 | −0.4 | −0.3 |
| PTAT | 0.47 | −0.14 | −0.23 | −0.04 |
| FUA | 0.55 | −0.19 | −0.43 | −0.1 |
| RUH | 0.99 | 0.85 | 0.4 | 1.24 |
| RUW | 0.68 | 0.44 | 0.35 | 0.99 |
| UCL | −0.01 | −0.46 | 0.13 | 0.03 |
| UDP | 0.09 | −0.9 | −0.69 | −0.5 |
| FTP | 0.46 | −0.2 | −0.02 | −0.08 |
| TLG | −0.08 | 0.56 | 0.77 | 0.1 |
| FLS | 0.46 | −0.71 | −0.15 | −0.24 |
| RTP | 0.44 | −0.13 | 0.28 | 0.3 |
| SCE | 1.6 | 1.4 | 1 | 1.2 |
| SSB | 5.3 | 5.8 | 4.9 | 5 |
| DSB | 4.3 | 4.6 | 3 | 3.1 |
| DCE | 1.7 | 1.6 | 1 | 1.1 |
| CM | 948 | 1110 | 1072 | 1164 |
| FM | 892 | 1034 | 1007 | 1079 |
| GM | 860 | 1062 | 1001 | 1124 |
| chip | GM3 | GM3 | GM3 | GM3 |
| imputed_call_rate | 99.9 | 99.9 | 99.9 | 99.9 |
| HBR | T | T | T | T |
| HDR | T | T | T | T |
| HH0 | T | T | T | T |
| HH1 | T | T | T | T |
| HH2 | T | T | T | T |
| HH3 | T | T | T | T |
| HH4 | T | T | T | T |
| HH5 | T | T | T | T |
| HHB | T | T | T | T |
| HHC | T | T | T | T |
| HHD | T | T | T | T |
| HHM | T | T | T | T |
| HHP | T | T | T | T |
| HHR | T | T | T | T |
| HCD | 0 | 0 | 0 | 0 |
| MGS | HO8403141494296 | HO8403201863001 | HO8403204164778 | HO8403204164778 |
| UDC | 0.8 | 0.45 | 0.19 | 0.72 |
| FLC | 0.77 | −0.37 | 0.07 | 0.01 |
| TPI | 2820 | 2862 | 2862 | 2973 |
| GL | −1.3 | 0.1 | −1.3 | −1 |
| MFV | 0.1 | 0.1 | 0.1 | 0.1 |
| DAB | 1 | 0.6 | 0.3 | 0.8 |
| KET | 2.4 | 1.9 | 1.2 | 2 |
| MAS | 1.1 | 0.4 | 2.9 | 0.3 |
| MET | 1.5 | 2.7 | 1.9 | 2.2 |
| RPL | 0.3 | 0.8 | 0.1 | 0.1 |
| HH6 | T | T | T | T |
| EFC | 2.7 | 2.6 | 2 | 2.1 |
| BWC | −0.57 | −2.37 | −2.09 | −2.56 |
| FE | 226 | 290 | 268 | 308 |
| FI | 0.1 | 0.5 | 0.1 | 0.6 |
| HLV | 0.5 | 0.5 | 0.9 | 0.4 |
| FS | 125 | 419 | 237 | 301 |
| RFI | −37 | −60 | 80 | 89 |
| | id17 | | | |
| | HO840003236266757 | HO840003236266758 | HO840003236266759 | HO840003236266760 |
| | sex | | | |
| | M | M | M | M |
| sire17 | HO840003204165457 | HO840003150687531 | HO840003150687531 | HO840003150687531 |
| dam17 | HO840003223118782 | HO840003204166547 | HO840003204166547 | HO840003220285661 |
| NM | 997 | 971 | 996 | 1023 |
| PL | 3.6 | 5.9 | 5.8 | 6.2 |
| SCS | 3 | 2.86 | 2.76 | 2.82 |
| DPR | −1.5 | −1.8 | −1.4 | −0.8 |
| NM_REL | 71 | 72 | 72 | 71 |
| Milk | 698 | 1659 | 1363 | 1658 |
| Fat | 120 | 88 | 95 | 94 |
| Pro | 46 | 53 | 53 | 63 |
| FatPct | 0.32 | 0.08 | 0.15 | 0.1 |
| ProPct | 0.08 | 0.01 | 0.04 | 0.04 |
| HCR | 1.3 | 0.3 | −0.7 | −1.5 |
| CCR | −0.6 | −0.5 | −0.7 | 0.5 |
| LIV | 2.4 | 3 | 4.7 | 2.6 |
| Milk_REL | 78 | 79 | 79 | 78 |
| STA | −0.02 | −1.75 | −0.68 | −0.87 |
| STR | −0.73 | −1.28 | −0.55 | −0.78 |
| BDE | −0.19 | −1.02 | −0.36 | −0.61 |
| DFM | 1.62 | 0.48 | 0.55 | 0.81 |
| RPA | −0.67 | −1.39 | −0.27 | 0.97 |
| TRW | 0.4 | −0.97 | −0.09 | 0.07 |
| RLS | 1.06 | −0.28 | −0.56 | −0.81 |
| RLR | 0.35 | 0.23 | 0.12 | −0.25 |
| FTA | −0.16 | 0.03 | 0.03 | −0.09 |
| PTAT | 1.15 | 0.22 | 0.34 | 0.22 |
| FUA | 0.63 | 0.23 | 0.12 | 0.03 |
| RUH | 1.33 | 0.85 | 0.97 | 1.09 |
| RUW | 1.3 | 0.47 | 0.91 | 1.12 |
| UCL | 0.96 | 0.07 | 0.1 | 0.2 |
| UDP | 0.44 | −0.46 | −0.29 | −0.47 |
| FTP | 1.39 | 0.51 | 0.52 | 0.45 |
| TLG | −0.34 | −0.07 | 0.08 | −0.16 |
| FLS | 0.47 | 0.07 | −0.09 | −0.21 |
| RTP | 1.51 | 0.55 | 0.55 | 0.61 |
| SCE | 1.7 | 1.9 | 1.6 | 1.8 |
| SSB | 5.8 | 6 | 5.7 | 5.5 |
| DSB | 4.1 | 6 | 4.6 | 4.5 |
| DCE | 1.6 | 2.3 | 1.9 | 1.4 |
| CM | 1011 | 975 | 1007 | 1034 |
| FM | 880 | 951 | 931 | 956 |
| GM | 955 | 879 | 901 | 943 |
| chip | GM3 | GM3 | GM3 | GM3 |
| imputed_call_rate | 99.9 | 99.9 | 99.9 | 99.9 |
| HBR | T | T | T | T |
| HDR | T | T | T | T |
| HH0 | T | T | T | T |
| HH1 | T | T | T | T |
| HH2 | T | T | T | T |
| HH3 | T | T | T | T |
| HH4 | T | T | T | T |
| HH5 | T | C | C | T |
| HHB | T | T | T | T |
| HHC | T | T | T | T |
| HHD | T | T | T | T |
| HHM | T | T | T | T |
| HHP | T | T | T | T |
| HHR | T | T | T | T |
| HCD | 0 | 0 | 0 | 0 |
| MGS | HO8403204165191 | HO8403141494296 | HO8403141494296 | HO8403204165493 |
| UDC | 1.05 | 0.73 | 0.63 | 0.7 |
| FLC | 0.42 | 0.5 | 0.1 | −0.05 |
| TPI | 2907 | 2772 | 2833 | 2871 |
| GL | −2.6 | 0.1 | −0.3 | −1.5 |
| MFV | 0.2 | 0.2 | 0.1 | 0.2 |
| DAB | 0.9 | 1.1 | 1.1 | 0.7 |
| KET | 1.6 | 2.4 | 2.5 | 2 |
| MAS | 0.9 | 0.2 | 2.1 | 2.1 |
| MET | 0.9 | 1.7 | 1.5 | 1.8 |
| RPL | −0.4 | 0.4 | 0.5 | −0.2 |
| HH6 | T | T | T | T |
| EFC | 3.6 | 0.2 | 0 | 0.3 |
| BWC | −1.24 | −1.8 | −0.86 | −1.18 |
| FE | 297 | 264 | 258 | 269 |
| FI | −0.6 | −1.3 | −1.1 | −0.6 |
| HLV | 1 | 0.4 | 0.6 | 1 |
| FS | 277 | 312 | 167 | 118 |
| RFI | −89 | −39 | −37 | 61 |

TABLE 7
Predicted Phenotypic Traits from Genotype (female cell lines)

| id17 | HO840003236266755 | HO840003236266756 | HO840003236266762 | HO840003236266764 |
|---|---|---|---|---|
| sex | F | F | F | F |
| sire17 | HO840003150687531 | HO840003150687531 | HO840003150687531 | HO840003150687531 |
| dam17 | HO840003214630863 | HO840003214630863 | HO840003214630863 | HO840003214630863 |
| NM | 1213 | 1139 | 1051 | 1050 |
| PL | 6.6 | 5.2 | 5.8 | 5.9 |
| SCS | 2.81 | 2.75 | 2.75 | 2.73 |
| DPR | 0 | −0.8 | −0.2 | 0.3 |
| NM_REL | 71 | 71 | 71 | 71 |
| Milk | 1343 | 1100 | 982 | 1151 |
| Fat | 116 | 127 | 106 | 99 |
| Pro | 61 | 53 | 51 | 49 |
| FatPct | 0.22 | 0.29 | 0.24 | 0.19 |
| ProPct | 0.07 | 0.06 | 0.07 | 0.05 |
| HCR | 1.1 | −0.1 | 1.3 | 1.9 |
| CCR | 1.3 | 0.2 | 1.1 | 1.3 |
| LIV | 3.6 | 2.8 | 2.9 | 4.4 |
| Milk_REL | 78 | 78 | 78 | 78 |
| STA | −2.18 | −1.28 | −1.27 | −1.51 |
| STR | −1.97 | −1.51 | −1.1 | −1.56 |
| BDE | −1.62 | −0.97 | −0.57 | −1.23 |
| DFM | 0.54 | 1.14 | 1.16 | 0.7 |
| RPA | 0.26 | −0.61 | 0.47 | −0.47 |
| TRW | −1.67 | −1.32 | −0.75 | −0.97 |
| RLS | 0 | 0.01 | −0.14 | −0.18 |
| RLR | −0.92 | −0.45 | −0.52 | −0.58 |
| FTA | −0.94 | −0.55 | −0.61 | −0.76 |
| PTAT | −0.41 | 0.2 | 0.03 | −0.51 |
| FUA | −0.47 | 0.22 | −0.34 | −0.48 |
| RUH | 0.46 | 1.28 | 0.77 | 0.37 |
| RUW | 0.44 | 0.98 | 0.83 | 0.29 |
| UCL | −0.31 | −0.35 | −0.11 | −0.23 |
| UDP | −1.16 | −0.41 | −1.07 | −0.82 |
| FTP | 0.13 | −0.31 | −0.1 | −0.28 |
| TLG | −0.36 | 0.78 | 0.45 | 0.35 |
| FLS | −0.77 | −0.3 | −0.36 | −0.54 |
| RTP | 0.19 | −0.21 | 0.14 | 0.05 |
| SCE | 1.1 | 1.5 | 1.4 | 1.1 |
| SSB | 4.8 | 5.2 | 5.3 | 5 |
| DSB | 4 | 3.7 | 4.1 | 3.3 |
| DCE | 1.5 | 1.6 | 1.6 | 1.5 |
| CM | 1228 | 1155 | 1068 | 1064 |
| FM | 1110 | 1037 | 942 | 974 |
| GM | 1171 | 1096 | 1003 | 1009 |
| chip | GM3 | GM3 | GM3 | GM3 |
| imputed_call_rate | 99.9 | 99.9 | 99.9 | 99.9 |
| HBR | T | T | T | T |
| HDR | T | T | T | T |
| HH0 | T | T | T | T |
| HH1 | T | T | T | T |
| HH2 | T | T | T | T |
| HH3 | T | T | T | T |
| HH4 | T | T | T | T |
| HH5 | T | T | T | T |
| HHB | T | T | T | T |
| HHC | T | T | T | T |
| HHD | T | T | T | T |
| HHM | T | T | T | T |
| HHP | T | T | T | T |
| HHR | T | T | T | T |
| HCD | 0 | 0 | 0 | 0 |
| MGS | HO8403204164778 | HO8403204164778 | HO8403204164778 | HO8403204164778 |
| UDC | 0.34 | 0.69 | 0.34 | 0.18 |
| FLC | −0.38 | −0.08 | −0.15 | −0.27 |
| TPI | 2966 | 2966 | 2877 | 2848 |
| GL | −0.3 | −0.1 | −0.1 | −1 |
| MFV | 0 | 0.1 | 0.1 | 0.1 |
| DAB | 0.4 | 0.4 | 0.5 | 0.6 |
| KET | 1.5 | 1.4 | 1.2 | 2 |
| MAS | 1.7 | 1.8 | 1.9 | 1.3 |
| MET | 2.3 | 2.2 | 2.2 | 2.2 |
| RPL | 0.3 | 0 | 0.2 | 0.1 |
| HH6 | T | T | T | T |
| EFC | 0.5 | −0.8 | 2.2 | 3.5 |
| BWC | −2.59 | −2.22 | −1.8 | −2.06 |
| FE | 326 | 321 | 277 | 264 |
| FI | 0.3 | −0.6 | 0.3 | 0.9 |
| HLV | 0.3 | 0.1 | 0.7 | 0.6 |
| FS | 358 | 290 | 218 | 228 |
| RFI | 35 | 46 | 55 | 86 |

These data illustrate that embryonic stem cells of the present teachings can be genotyped in manners that are common in the art.

### Example 33

This example illustrates induction of primordial germ cell-like cells (PGCLCs) from bovine embryonic stem cells of the present teachings.

Some culture methods in this example use GK15 medium. GK15 medium (GBM) was made by adding 404 mL Glasgow's minimal essential medium (GMEM; Gibco, 11710035), 75 mL of KNOCKOUT™ Serum Replacement (KSR; Gibco, 10828-010, Trademark Thermofisher Scientific, Waltham, MA), 5 ml 2 mM L-glutamine, 1 mL 2-mercaptoethanol, 5 mL non-essential amino acids (NEAA, 100×), 5 mL sodium pyruvate, and 5 mL Penicillin-Streptomycin Solution (P/S, 100×).

The present inventors first induced the bovine embryonic stem cells into progenitor cells—specifically bovine formative-like cells or bovine mesoderm-like cells. To prepare the progenitor cells, 16.7 ug/ml of human plasma fibronectin (Millipore, FC010) in PBS was used to coat cell culture plates which were then incubated for 1 hour at 37° C. The fibronectin solution was removed. 1 ml/well of bFCM or bMLC medium (see below) was added to the coated plate, which was then incubated for 1 hour at 37° C. in a 5% O₂, 5% CO₂ incubator.

The induction medium was different for each of the progenitor cells. For bovine formative cells, bFCM was made by supplementing 10 ml NBM (see Example 22) with 10 ng/ml bFGF, 10 ng/ml activin A, and 3 μM CHIR99021. For bovine mesoderm-like cell induction, 10 ml of GK15 medium was supplemented to a final concentration of 70 ng/ml of activin A and 3 μM CHIR99021.

Feeder free bovine embryonic stem cells were then washed with PBS. 500 μl of 0.25% Trypsin/EDTA was added to each well and the cells were incubated at 37° C. for 3 min. 500 μl of NBM was added and pipetted up and down. The cell suspension was then transferred to a 15 ml tube and centrifuged at 300×g, 20° C., for 5 min. The medium was removed and the cell pellets were resuspended in 1 ml of NBM. 1.0-2.0×10⁵ cells per well were added into the plate containing bFCM or bMLC medium prepared above. The cells were cultured for 24 to 48 hrs at 37° C. in a 5% O₂, 5% CO₂ incubator.

The medium for bovine PGCLCs was GBM supplemented with 1000 U/mL of rLIF, 100 ng/mL of SCF, 50 ng/mL of EGF, and 500 ng/ml of BMP4. 200 μl of this medium was added to a 96 well u-bottom plate coated in fibronectin as described above.

The progenitor cells were then washed with PBS. 500 μl of 0.25% Trypsin/EDTA was added to each well and the cells were incubated at 37° C. for 3 min. 500 μl of NBM was added and pipetted up and down. The cell suspension was then transferred to a 15 ml tube and centrifuged at 300×g, 20° C., for 5 min. The medium was then removed. The cell pellets were resuspended in 1 ml of GBM. 4×10³ cells per well were then added to each well of the 96 well u-bottom plate containing PGCLC medium. The cells were then maintained for 96 hours at 37° C. in a 5% O₂, 5% CO₂ incubator. Fresh bovine PGCLC medium was added every other day.

### Example 34

This example illustrates the characterization of PGCLCs as induced in Example 33.

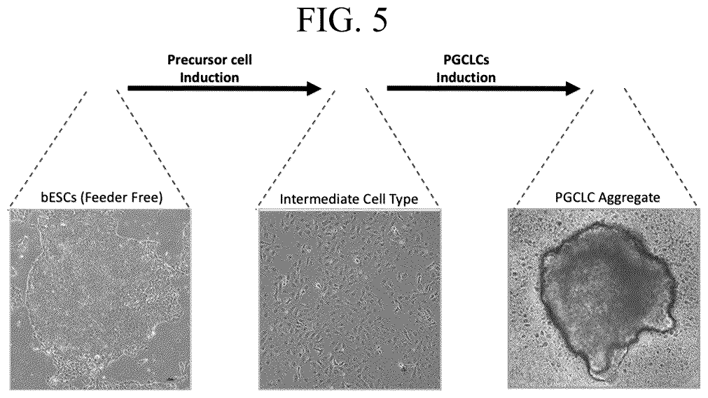

Feeder free embryonic stem cells, intermediate cells, and induced PGCLCs were all observed under a Nikon ECLIPSE Ti2 inverted microscope at 40×. (FIG. 5). All cells had the expected morphology.

RT-PCR primers were designed for pluripotency genes (OCT4; see Table 2) and PGC specification genes (NANOS3, SOX17, TBXT, and TFAP2C). The primers for the specification genes are listed in Table 8.

TABLE 8
RT-PCR Primers for PGC Specification Genes

| Genbank | Target | | | Product |
|---|---|---|---|---|
| Accession | Name | SEQ ID NO | Sequence | Size |
| XM_019964015.1 | NANOS3 | 19 | CTCCGTCTACAGCTACACCA | |
| | | 20 | AGGGAGGAGTTCCAGAAGAC | 142 |
| NM_001206251.1 | SOX17 | 21 | CCGAGTTGAGCAAGATGCTG | |
| | | 22 | CGGTACTTGTAGTTGGGATGG | 126 |
| >NM_001192985.1 | TBXT | 23 | TACATAAGTATGAGCCTCGAATCC | |
| | | 24 | TCTTTGTGATCGCTTCTTT | 196 |
| NM_001075509.1 | TFAP2C | 25 | ATTCGCAAAGGTCCTATTTCCA | |
| | | 26 | TAGATGTAGAGCTGAGGAGGGA | 139 |

RT-qPCR was performed as in Example 29 to look at gene expression in bovine embryonic stem cells (bESC), bovine PGCLCs induced from bovine formative cells, and bovine PGCLCs induced from bovine mesoderm-like cells. Within an extract, mRNA expression for each marker was normalized to GADPH and then compared to expression in bESCs. Expression relative to expression in bESCs is shown in Table 9.

TABLE 9
RT-PCR Expression in Bovine Primordial Germ Cell-Like Cells

| | bESC | | bPGCLC-bFCM | | bPGCLC-bMLC | |
|---|---|---|---|---|---|---|
| | mean | SD | mean | SD | mean | SD |
| OCT4 | 1 | 0 | 1.388 | 0.00184 | 1.004 | 0.00722 |
| NANOS3 | 1 | 0 | 14.431 | 0.499 | 38.132 | 1.927 |
| SOX17 | 1 | 0 | 10.977 | 0.405 | 13.552 | 0.437 |
| TBXT | 1 | 0 | 1276.129 | 0.926 | 2088.054 | 1.075 |
| TFAP2C | 1 | 0 | 154.943 | 0.0629 | 184.566 | 0.138 |

Transcript abundance of pluripotency marker OCT4 is unchanged, but transcript abundance in PGC specification markers NANOS3, SOX17, TBXT, and TFAP2C are all markedly increased.

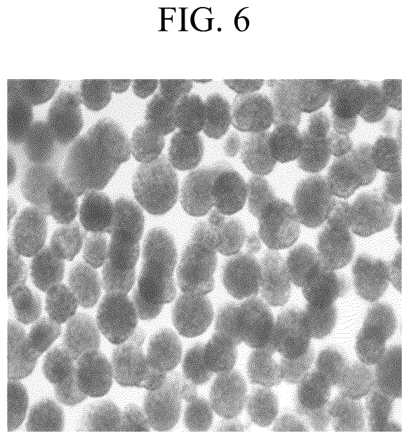

Further, aggregates of induced PGCL cells had the expected cell morphology (see FIG. 6.)

These data illustrate induction of bovine primordial germ cell-like cells from bovine embryonic stem cells.

### Example 35

This example illustrates the production of porcine embryonic stem cells.

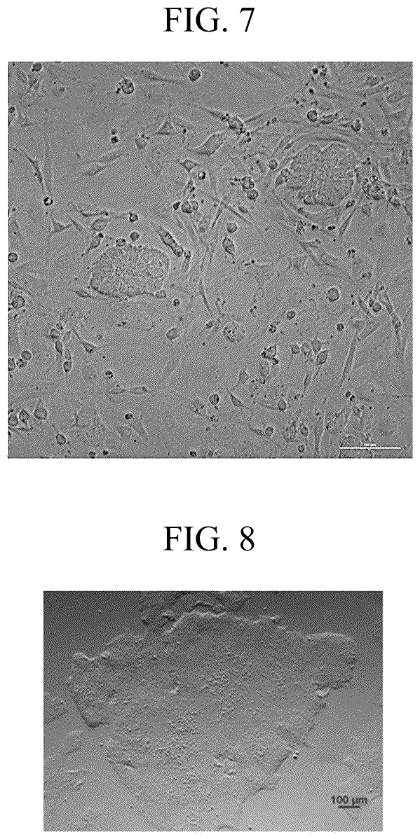

Porcine embryonic stem cells were prepared from in vitro fertilized pig oocytes as described in Example 23 with the following changes: cells were grown on CF1 feeder cells in N2B27 medium (see Example 22) supplemented with 10 ng/ml bFGF, 3 μM CHIR99021, 1 μM PD0325901 (ROCK inhibitor), and 10 ng/ml activin A. The PD0325901 was withdrawn after six days. Cells were observed under a Nikon ECLIPSE Ti2 inverted microscope at 40×. (Photographs were taken with NIS elements Br software.) FIG. 7 shows these cells at 40× magnification. The cells were then transferred to cell free culture as described in Example 25, except that the ES medium comprised N2B27 medium supplemented with 10 ng/ml bFGF, 3 μM CHIR99021, 1 μM PD0325901 (ROCK inhibitor), and 10 ng/ml activin A. The PD0325901 was withdrawn after six days. Feeder free cells are shown at 40× in FIG. 8. The scale bar is 100 μm. The embryonic stem cells gather in aggregate spots when grown with feeder cells (FIG. 7). When grown under feeder free conditions, the cells form a confluent structure (FIG. 8). This morphology is expected for embryonic stem cells.

### Example 36

This example illustrates behavior of porcine embryonic stem cells that is typical of pluripotent cells.

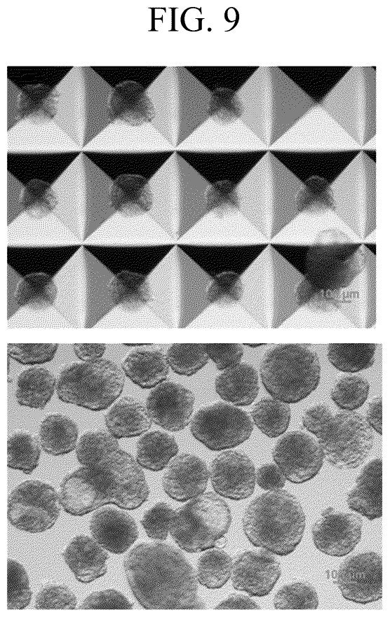

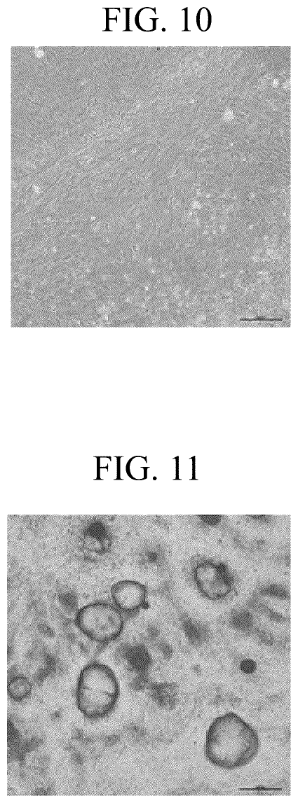

Formation of embryoid bodies and spontaneous differentiation of porcine embryonic stem cells was carried out as described for the bovine embryonic stem cells in Example 30. Porcine embryonic stem cells formed embryoid bodies (FIG. 9), mesodermal cells (FIG. 10), and endodermal cells (FIG. 11). These data indicate that the porcine embryonic stem cells are pluripotent.

### Example 37

This example illustrates the pluripotency of porcine embryonic stem cells.

RNA prep and RT-qPCR will be performed for the porcine embryonic stem cells and the data analyzed as in Example 29. The primers for pluripotency markers are listed in Table 10.

TABLE 10
Primers for Pluripotency Markers in Porcine

| Genbank | Target | | Sequence | Product |
|---|---|---|---|---|
| Accession | Name | SEQ ID NO | | Size |
| NM_001129971 | NANOG | 27 | TGAAATGTAAGAGGTGGCAGAA | |
| | | 28 | GCATCCTTGGTGGTAGGAATAG | 110 |
| NM_001113060.1 | OCT_4 | 29 | TTTGCAGCTCAGTTTCAAGAAC | |
| | | 30 | GGTCTCTGCCTTGCATATCTC | 109 |
| NM_001123197 | SOX2 | 31 | GCAGTACAACTCCATGACCA | |
| | | 32 | CATGGAACCGAGCGTCAT | 106 |
| NM_001278769 | CDX2 | 33 | TCGGCAGCCAAGTGAAA | |
| | | 34 | CTACAGTCGCTACATCACCAT | 109 |
| NM_214328.2 | GATA6 | 35 | GGCCTCTACAGCAAGATGAAC | |
| | | 36 | GACAGTTGGCACAGGACAAT | 100 |
| NM_001206359.1 | GAPDH | 37 | TCAAGAAGGTGGTGAAGCAG | |
| | | 38 | AGAGTGAGTGTCACTGTTGAAG | 104 |

It is expected that pluripotency markers OCT4, SOX2, and NANOG will be upregulated relative to fibroblast cells, but that differentiation markers CDX2 and GATA4 will not.

### Example 38

This example illustrates the use of bovine ESCs for the production of cultured beef.

Bovine embryonic stem cells as grown in Example 26 may be treated with 20 ng/ml bFGF and 20 ng/ml EGF for approximately 16 hours followed by a media change. The cells will then be grown on the same medium, with daily changes for 10-15 days to induce muscle cell differentiation. The cells will then be transitioned to 3-dimensional culture on a porous, edible scaffold. It is expected that the cells will then grow on the scaffold in a manner approximating skeletal muscle, and therefore beef.

### Example 39

This example illustrates the use of porcine ESCs for the production of cultured pork.

Porcine embryonic stem cells as grown in Example 35 may be treated with 20 ng/ml bFGF and 20 ng/ml EGF for approximately 16 hours followed by a media change. The cells will then be grown on the same medium, with daily changes for 10-15 days to induce muscle cell differentiation. The cells will then be transitioned to 3-dimensional culture on a porous, edible scaffold. It is expected that the cells will then grow on the scaffold in a manner approximating skeletal muscle, and therefore pork.

### Example 40

This example illustrates the use of bovine ESCs for the production of organ meats.

Bovine embryonic stem cells as grown in Example 26 may be treated with 100 ng/ml activin A and 3 μM CHIR99021 on feeder free plates with daily media changes for 2 days to create endoderm-like cells. The cells will then be treated with 5 ng/ml bFGF and 20 ng/ml BMP4 for 5 days with daily medium changes to induce hepatic endoderm cells. These cells will then be treated with 20 ng/ml hepatocyte growth factor (HGF) for 5 days with daily medium changes to induce immature hepatocyte-like cells. The cells will then be treated with 20 ng/ml HGF, 20 ng/ml Oncostatin M, and 100 nM dexamethasone for 10-12 days. The cells will then be adapted to 3-dimensional culture on a porous, edible scaffold and allowed to grow to confluence. The cells are expected to form a tissue similar to beef liver at this stage.

All publications cited herein are incorporated by reference, each in their entirety.

1. A method of livestock breeding comprising:
A) creating at least one embryo;
B) creating lines of embryonic stem cells from the at least one embryo;
C) genotyping the at least one embryo or the embryonic stem cell lines derived therefrom;
D) selecting at least one embryonic stem cell line for breeding;
E) inducing embryonic stem cells from at least one selected embryonic stem cell line to differentiate into gamete or gamete-like cells; and
F) combining the gametes or gamete-like cells with opposite sex gametes or gamete-like cells to produce at least one next generation embryo.

2. (canceled)

3. (canceled)

4. The method according to claim 1, wherein the creating at least one embryo in step A comprises mixing gametes of livestock parents of high genetic merit or the opposite sex gamete-like cells in step F are from an opposite sex genotype embryonic stem cell line produced in step B.

5. The method according to claim 1, further comprising:
G) creating an embryonic stem cell line from the at least one next generation embryo to create next generation embryonic stem cell lines; and
H) repeating steps C-F on the next generation embryonic stem cell lines.

6. The method according to claim 5, further comprising producing animals from at least one of the embryonic stem cell lines from step B or step G.

7. The method according to claim 1, wherein the at least one embryo is a bovine embryo or a porcine embryo.

8. The method according to claim 1, wherein the creating lines of embryonic stem cells comprises administering to an embryo, a blastocyst derived therefrom, or a part thereof a WNT-signaling inhibitor, a GSK3 signaling inhibitor, a tankyrase inhibitor, a Rho-kinase (ROCK) inhibitor, an inducer of activin A signaling, or a combination thereof.

9. The method according to claim 8, wherein:
the WNT-signaling inhibitor, if present, is selected from the group consisting of iwr-1, iwr-1-endo, iwp-2, Box5, iCRT3, sclerostin, dkk2, dkk1, LF3, CCT036477, FH535, cardamonin, IWP-L6, Wnt-C59, niclosamide, XAV-939, ICG-001, LGK-974, CP21R7, NCB-0846, PNU-74654, salinomycin, KY021 11, WIKI4, PRI-724, KYA1797K, 2,4-diamino-quinazoline, Ant 1.4Br, Ant 1.40, apicularen, bafilomycin, ETC-159, G007-LK, G244-LM, IWR, NSC668036, PKF1 15-584, pyrvinium, quercetin, shizokaol D, BC2059, and a combination thereof;
the GSK3 inhibitor, if present, is selected from the group consisting of CHIR99021, AR-A 014418, A 1070722, SB 415286, TCS 2002, 3F8, TDZD 8, TC-G 24, BIO-acetoxime, indirubin-3′-oxime, TWS 119, TCS 21311, SB 216763, BIO, lithium carbonate, kenpaullone, alsterpaullone, and CHIR 98014;
the tankyrase inhibitor, if present, is selected from the group consisting of XAV939, AZ 6102, JW 55, TC-E 5001, and WIKI4;
the ROCK inhibitor, if present, is Y-27632 or PD0325901; and
the inducer of activin A signaling, if present is activin A, alantolactone, BMP4, nodal, stauprimide, SB-431542, A01, GDF8, or conophylline.

10. The method according to claim 8, wherein the cells are further treated with bFGF, FGF2, FGF4, or SUN 11602.

11. (canceled)

12. The method according to claim 1, wherein the inducing embryonic stem cells into gametes or gamete-like cells comprises inducing embryonic stem cells from each of the selected embryonic stem cell lines into progenitor cells by administering to the embryonic stem cells an inducer of activin A signaling, an inducer of basic fibroblast growth factor (bFGF) signaling, an inducer of insulin signaling, an inducer of WNT signaling, or a combination thereof.

13. The method according to claim 12, wherein the progenitor cells are mesoderm-like cells and the inducing comprises administering to the cells activin A, CHIR99021, and a serum replacement.

14. The method according to claim 12, wherein the progenitor cells are formative cell-like cells and the inducing comprises administering to the cells bFGF, activin A, and CHIR99021.

15. The method according to claim 12, wherein the inducing embryonic stem cells into gamete or gamete-like cells further comprises inducing the progenitor cells into primordial germ cell-like cells (PGCLCs) by administering to the cells: an inducer of BMP signaling, an inducer of LIF signaling, an inducer of SCF signaling, an inducer of EGF signaling, an inducer of WNT signaling, or a combination thereof.

16. The method according to claim 15, wherein the inducing the progenitor cells into PGCLCs comprises administering LIF, SCF, EGF, and BMP4.

17. The method according to claim 16, further comprising administering to the progenitor cells a serum replacement.

18. The method according to claim 1, wherein the inducing embryonic stem cells into gamete or gamete-like cells comprises inducing the embryonic stem cells into spermatogonial stem cell-like cells (SSCLC) by administering to the embryonic stem cells an inducer of retinoic acid (RA) signaling, an inducer of Glial Cell Derived Neurotrophic Factor (GDNF) signaling, an inducer of testosterone signaling, or a combination thereof.

21. The method according to claim 1, wherein the inducing embryonic stem cells into gamete or gamete-like cells comprises inducing the embryonic stem cells into spermatids by administering to the embryonic stem cells an inducer of insulin signaling, an inducer of testosterone signaling, an inducer of follicle stimulating hormone (FSH), Bovine Pituitary Extract (BPE), or a combination thereof.

22. The method according to claim 1, wherein the inducing embryonic stem cells into gamete or gamete-like cells comprises inducing the embryonic stem cells into oocytes by:
inducing the embryonic stem cells into primordial germ cell-like cells;
placing the primordial germ cell-like cells in a reconstituted ovary;
administering to the cells in the reconstituted ovary an estrogen receptor agonist;
administering to the cells in the reconstituted ovary at least one inducer of TGFβ signaling; and
administering to the cells in the reconstituted ovary an inducer of FSH signaling, an inducer of EGF signaling, an inducer of gonadotropin signaling, or a combination thereof.

23-29. (canceled)

30. A method of generating bovine primordial germ cell-like cells in vitro comprising:
administering to bovine embryonic stem cells an inducer of FGF signaling, an inducer of activin A signaling, and an inducer of WNT signaling to induce bovine formative cells or administering to bovine embryonic stem cells an inducer of activin A signaling, an inducer of WNT signaling, and a serum replacement to induce bovine mesoderm-like cells; and
administering to the bovine formative cells or the bovine mesoderm-like cells an inducer of LIF signaling, an inducer of SCF signaling, an inducer of EGF signaling, and an inducer of BMP signaling to induce bovine primordial germ cell-like cells.

31. The method according to claim 30, comprising inducing bovine formative cells and wherein the inducer of WNT signaling induces WNT by inhibiting GSK3.

32. The method according to claim 30, comprising inducing bovine formative cells wherein the inducer of activin A signaling is activin A, alantolactone, BMP4, nodal, stauprimide, SB-431542, A01, GDF8, or conophylline, the inducer of FGF signaling is bFGF, SUN 11602, sucralfate, or FGF-P, and the inducer of WNT signaling is WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5.

33. The method according to claim 30, comprising inducing bovine mesoderm-like cells, wherein the inducer of activin A signaling is activin A, alantolactone, BMP4, nodal, stauprimide, SB-431542, A01, GDF8, or conophylline, and the inducer of WNT signaling is WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5.

34. The method according to claim 30, wherein the inducer of LIF signaling is LIF, the inducer of SCF signaling is SCF, and the inducer of BMP signaling is BMP4, BMP8b, BMP7, TGFβ, sb4 (2-[[(4-Bromophenyl)methyl]thio]benzoxazole), triamcinolone, isoliquiritigenin and, km11073, 4′-hydroxychalcon, or SVAK-12.

35. The method of claim 30 wherein:
the inducer of FGF signaling is bFGF, the inducer of activin A signaling is activin A, and the inducer of WNT signaling is CHIR99021;
the inducer of LIF signaling is rLIF, the inducer of SCF signaling is SCF, the inducer of EGF signaling is EGF, the inducer of BMP signaling is BMP4, and the administering to the bovine formative cells or the bovine mesoderm-like cells further comprises administering a serum replacement to produce bPGCLCs.

36. A method of inducing porcine embryonic stem cells comprising:
providing an embryo, blastocyst, or part thereof;
administering to the embryo, blastocyst, or part thereof: an FGF signaling inducer, a WNT signaling inducer, a Rho-kinase (ROCK) inhibitor, and an activin A signaling inducer.

37. The method according to claim 36, wherein the inducer of FGF signaling is bFGF, SUN 11602, sucralfate, or FGF-P, the inducer of WNT signaling is WNT2b, WNT3A, WNT-4, Wnt5A, Wnt5b, Wnt7a, Wnt8a, Wnt9a, Wnt-9b, Wnt10b, Wnt11, Wnt16b, norrin, Wnt Agonist I, Wnt Agonist II, CHIR99021, LP 922056, BML 284, WAY 316606 hydrochloride, SGC AAK1 1, CHIR 98014, or Foxy 5, the ROCK inhibitor is Y-27632 or PD0325901, and the inducer of activin A signaling is activin A, alantolactone, BMP4, nodal, stauprimide, SB-431542, A01, GDF8, or conophylline.

38. The method according to claim 36, wherein the inducer of WNT signaling induces WNT by inhibiting GSK3.

39. The method according to claim 36, wherein the inducer of FGF signaling is bFGF, the inducer of WNT signaling is CHIR99021, the ROCK inhibitor is PD0325901, and the inducer of activin A signaling is activin A.

40. (canceled)

41. The method according to claim 4, wherein:
the livestock parents are bovine parents;
the creating embryonic stem cells in step B) comprises administering FGF2 and IWR1 to the at least one embryo, a blastocyst matured therefrom, or a part thereof;
the inducing embryonic stem cells to differentiate into gametes or gamete-like cells in step E) comprises:
i) inducing the embryonic stem cells into formative cells by administering activin A, bFGF, and CHIR99021 or inducing the stem cells into mesoderm-like cells by administering serum replacement, activin A, and CHIR99021;
ii) inducing the formative cells or mesoderm-like cells into PGCLCs by administering BMP4, LIF, SCF, EGF, and a serum replacement;
iii) inducing the PGCLCs that have an XY genotype into spermatid-like cells comprising:
a) inducing the PGCLCs that have an XY genotype into SSCLs by administering RA, GDNF, and testosterone;
b) inducing the SSCLCs into spermatid-like cells by administering serum replacement, testosterone, and BPE;
iv) inducing the PGCLCs that have an XX genotype into oocytes comprising:
a) mixing the XX PGCLS with female bovine gonadal somatic cells and RA to form a reconstituted ovary;
b) administering to the reconstituted ovary ICI182780 to induce secondary follicle-like structures (2FLs);
c) administering to the 2FLs FSH, BMP15, and GDF9 to induce cumulus-oocyte complexes (COCs);
d) administering to the COCs FSH, EGF, and hCG to form oocytes;
wherein the combining the gametes or gamete-like cells in step F) comprises performing in vitro fertilization with the gametes or gamete-like cells to form a plurality of next generation embryos; and
further comprising:
G) creating an embryonic stem cell line from each of the plurality of next generation embryos to create next generation embryonic stem cell lines; and
H) repeating steps C-F on the next generation embryonic stem cell lines.

42. The method according to claim 4 wherein:
the at least one livestock parent is a porcine parent;
wherein the creating in step B) comprises administering bFGF, CHIR99021, and PD0325901 to the embryo, blastocyst matured therefrom, or part thereof;
wherein the inducing in step E) comprises:
i) inducing the embryonic stem cells into formative cells by administering activin A, bFGF, and CHIR99021 or inducing the embryonic stem cells into mesoderm-like cells by incubating in a medium comprising serum replacement, activin A, and CHIR99021;
ii) inducing the formative cells or mesoderm-like cells into PGCLCs by administering to the cells BMP4, LIF, SCF, EGF, and a serum replacement;
iii) inducing the PGCLCs that have an XY genotype into spermatid-like cells comprising:
a) inducing the PGCLCs that have an XY genotype into SSCLC by administering to the cells RA, GDNF, and testosterone;
b) inducing the SSCLCs into spermatid-like cells by administering to the cells a serum replacement, testosterone, and BPE;
iv) inducing the PGCLCs that have an XX genotype into oocytes comprising:
a) mixing the PGCLCs that have an XX genotype with female bovine gonadal somatic cells and RA to form a reconstituted ovary;
b) administering to the reconstituted ovary ICI182780 to induce secondary follicle-like structures (2FLs);
c) administering to the 2FLs FSH, BMP15, and GDF9 to induce cumulus-oocyte complexes (COCs);
d) administering to the COCs FSH, EGF, and hCG to form oocytes;
e) maturing the oocytes into MII oocytes
wherein the combining in step F) comprises performing in vitro fertilization with the gametes or gamete-like cells to form a plurality of next generation embryos; and
further comprising:
G) creating an embryonic stem cell line from each of the plurality of next generation embryos to create next generation embryonic stem cell lines; and
H) repeating steps C-F on the next generation embryonic cell lines.


SEQUENCE LISTING (38 sequences)

SEQ ID NO: 1 - DNA, 20 bp, Bos taurus
cgtctggagctggagaagga

SEQ ID NO: 2 - DNA, 17 bp, Bos taurus
cggccagttcggctttc

SEQ ID NO: 3 - DNA, 26 bp, Bos taurus
gagcagccggaggagatgtaccagac

SEQ ID NO: 4 - DNA, 26 bp, Bos taurus
ggctcaggccagggccagggcgcacc

SEQ ID NO: 5 - DNA, 23 bp, Bos taurus
aaagttacgtgtccttgcaaacg

SEQ ID NO: 6 - DNA, 23 bp, Bos taurus
gaggagggaagaggagagacagt

SEQ ID NO: 7 - DNA, 20 bp, Bos taurus
gttttgaggctttgcagctc

SEQ ID NO: 8 - DNA, 20 bp, Bos taurus
cctgcagattctcgttgttg

SEQ ID NO: 9 - DNA, 20 bp, Bos taurus
agcgcatggacagctacgcg

SEQ ID NO: 10 - DNA, 20 bp, Bos taurus
atgggctgcatctgagcggc

SEQ ID NO: 11 - DNA, 21 bp, Bos taurus
tcatcatctctggaccttctg

SEQ ID NO: 12 - DNA, 19 bp, Bos taurus
atgccaaagtggtcatgga

SEQ ID NO: 13 - DNA, 21 bp, Bos taurus
cttacacaaagaaggctcctc

SEQ ID NO: 14 - DNA, 23 bp, Bos taurus
ctataataaggtcagccactccc

SEQ ID NO: 15 - DNA, 22 bp, Bos taurus
acaacttcatctttggtcagag

SEQ ID NO: 16 - DNA, 20 bp, Bos taurus
atccaagactgagtccacca

SEQ ID NO: 17 - DNA, 22 bp, Bos taurus
gcaccactctttctacaatgag

SEQ ID NO: 18 - DNA, 22 bp, Bos taurus
agtctcaaacataatctgggtc

SEQ ID NO: 19 - DNA, 20 bp, Bos taurus
ctccgtctacagctacacca

SEQ ID NO: 20 - DNA, 20 bp, Bos taurus
agggaggagttccagaagac

SEQ ID NO: 21 - DNA, 20 bp, Bos taurus
ccgagttgagcaagatgctg

SEQ ID NO: 22 - DNA, 21 bp, Bos taurus
cggtacttgtagttgggatgg

SEQ ID NO: 23 - DNA, 24 bp, Bos taurus
tacataagtatgagcctcgaatcc

SEQ ID NO: 24 - DNA, 19 bp, Bos taurus
tctttgtgatcgcttcttt

SEQ ID NO: 25 - DNA, 22 bp, Bos taurus
attcgcaaaggtcctatttcca

SEQ ID NO: 26 - DNA, 22 bp, Bos taurus
tagatgtagagctgaggaggga

SEQ ID NO: 27 - DNA, 22 bp, Sus scrofa
tgaaatgtaagaggtggcagaa

SEQ ID NO: 28 - DNA, 22 bp, Sus scrofa
gcatccttggtggtaggaatag

SEQ ID NO: 29 - DNA, 22 bp, Sus scrofa
tttgcagctcagtttcaagaac

SEQ ID NO: 30 - DNA, 21 bp, Sus scrofa
ggtctctgccttgcatatctc

SEQ ID NO: 31 - DNA, 20 bp, Sus scrofa
gcagtacaactccatgacca

SEQ ID NO: 32 - DNA, 18 bp, Sus scrofa
catggaaccgagcgtcat

SEQ ID NO: 33 - DNA, 17 bp, Sus scrofa
tcggcagccaagtgaaa

SEQ ID NO: 34 - DNA, 21 bp, Sus scrofa
ctacagtcgctacatcaccat

SEQ ID NO: 35 - DNA, 21 bp, Sus scrofa
ggcctctacagcaagatgaac

SEQ ID NO: 36 - DNA, 20 bp, Sus scrofa
gacagttggcacaggacaat

SEQ ID NO: 37 - DNA, 20 bp, Sus scrofa
tcaagaaggtggtgaagcag

SEQ ID NO: 38 - DNA, 22 bp, Sus scrofa
agagtgagtgtcactgttgaag